(19) United States
(12) Patent Application Publication
(10) Pub. No.: US 2022/0218598 A1
(43) Pub. Date: Jul. 14, 2022

(54) METHODS AND COMPOSITIONS FOR RAPID DELIVERY OF ANTI-SEIZURE THERAPEUTICS

(71) Applicant: Neurelis, Inc., San Diego, CA (US)

(72) Inventors: Sunita Misra, Chicago, IL (US); Enrique J. Carrazana, San Diego, CA (US); Adrian L. Rabinowicz, San Diego, CA (US); Stuart Madden, San Diego, CA (US); Edward T. Maggio, San Diego, CA (US)

(73) Assignee: Neurelis, Inc., San Diego, CA (US)

(21) Appl. No.: 17/570,535

(22) Filed: Jan. 7, 2022

Related U.S. Application Data
(60) Provisional application No. 63/284,033, filed on Nov. 30, 2021.
(60) Provisional application No. 63/276,761, filed on Nov. 8, 2021.
(60) Provisional application No. 63/276,760, filed on Nov. 8, 2021.
(60) Provisional application No. 63/135,291, filed on Jan. 8, 2021.

(51) Int. Cl. A61K 9/00; A61K 31/5513; A61K 31/7016; A61K 47/10; A61K 47/22

(52) U.S. Cl. CPC A61K 9/0043; A61K 31/5513; A61K 47/22; A61K 47/10; A61K 31/7016

(57) ABSTRACT

Compositions for intranasal delivery of benzodiazepines and methods for their use to treat and prevent seizures are provided. The compositions deliver rapid therapeutic onset with a decreased incidence and/or severity of adverse effects after administration. Additionally, use of the compositions for the treatment of seizure clusters increases the time to a second seizure and duration of the interseizure cluster interval.

### CROSS-REFERENCE TO RELATED APPLICATIONS

This application claims priority to U.S. Provisional Application No. 63/135,291 filed Jan. 8, 2021; U.S. Provisional Application No. 63/276,760 filed Nov. 8, 2021; U.S. Provisional Application No. 63/276,761 filed Nov. 8, 2021; and U.S. Provisional Application No. 63/284,033 filed Nov. 30, 2021. Each of these applications is hereby incorporated by reference in its entirety.

### FIELD

The present disclosure relates generally to compositions comprising a benzodiazepine and an alkyl maltoside, suitable for intranasal administration and effective to treat seizures and seizure disorders.

### BACKGROUND

Benzodiazepines have been identified as possessing sedative, tranquilizing, and muscle relaxing properties and, as such, are useful in preventing or treating seizures, seizure conditions (such as epilepsy), and the symptoms thereof.

Various formulations comprising benzodiazepines are currently available, such as oral, rectal, and parenteral formulations. The ability to utilize these types of formulations, however, has been significantly limited, for example, due to difficulty of administration (e.g., parenteral, rectal administration), and a pharmacokinetic profile insufficient to achieve a desired therapeutic effect, particularly when administered as a rescue therapeutic during an active seizure episode (e.g., oral administration via, e.g., VALIUM®). In orally administered formulations, the amount of time required for the systemic circulation of the benzodiazepine to reach therapeutically relevant concentrations in blood plasma is often an hour or more. Due to the nature of seizures and muscle spasms, it can be extremely difficult for either a patient or a care-giver to administer the benzodiazepine drug orally or rectally. Intravenous (IV) administration provides a faster route for achieving systemic therapeutic blood levels, however IV administration is generally limited to trained health care professionals in tightly controlled clinical settings.

What is needed is a benzodiazepine composition that can be easily administered, either by the subject themselves or by a caregiver who may not be medically trained, and effecting a high bioavailability of the benzodiazepine as well as a fast therapeutic effect to rapidly and effectively stop a seizure. Such a composition will additionally be well-tolerated by the subject, causing little to no adverse effects (AEs) that would result in discontinuation of the treatment of seizures with the composition.

### SUMMARY

In one aspect, the present disclosure provides a method of treating a seizure in a subject in need thereof. This method comprises intranasally administering a first dose of a composition comprising an effective amount of diazepam, an alkyl maltoside, and a carrier system comprising one or more natural or synthetic tocopherols or tocotrienols and one or more alcohols, to a nasal mucosal membrane of the subject.

In another aspect, the present disclosure provides a method of preventing a seizure in a subject. This method comprises administering a composition comprising an effective amount of diazepam, an alkyl maltoside, and a carrier system comprising one or more natural or synthetic tocopherols or tocotrienols and one or more alcohols to a nasal mucosal membrane of the subject during a prodromal or pre-ictal phase of the seizure.

In another aspect, the present disclosure provides a method of increasing the time to a second seizure in a subject suffering from recurrent seizures. This method comprises administering a composition comprising an effective amount of diazepam, an alkyl maltoside, and a carrier system comprising one or more natural or synthetic tocopherols or tocotrienols and one or more alcohols to a nasal mucosal membrane of the subject before, during, or after a first seizure, wherein administration of the composition increases the time to a second seizure in the subject.

In another aspect, the present disclosure provides a method of increasing the time to a seizure cluster in a subject suffering from recurrent seizure clusters. This method comprises administering a composition comprising an effective amount of diazepam, an alkyl maltoside, and a carrier system comprising one or more natural or synthetic tocopherols or tocotrienols and one or more alcohols to a nasal mucosal membrane of the subject before, during, or after a first seizure, wherein administration of the composition increases the length of an interseizure cluster interval (ISCI) in the subject.

### BRIEF DESCRIPTION OF THE FIGURES

FIG. 1 depicts the plasma concentrations versus time for subjects, illustrating that despite near-immediate therapeutic benefit post-administration, Tₘₐₓ is not achieved until at least an hour after administration.

FIG. 2A and FIG. 2B depict plasma concentrations of diazepam administered orally (10 mg), rectally (15 mg or 20 mg), and intranasally (15 mg or 20 mg). Notably, a later Tₘₐₓ is achieved in compositions administered intranasally.

FIG. 3A and FIG. 3B depict graphs comparing intranasally administered diazepam to rectally administered diazepam. FIG. 3A depicts 15 mg dosages, while FIG. 3B depicts 20 mg dosages.

FIG. 4 depicts the mean diazepam concentration curves (ng/mL) for non-seizure and seizure groups.

FIG. 5A and FIG. 5B depict mean diazepam concentration curves (ng/mL) broken out by age. FIG. 5A depicts the non-seizure group, while FIG. 5B depicts the seizure group.

FIG. 6 depicts mean Quality of Life in Epilepsy (QOLIE) scores for all data from a long-term safety study of diazepam nasal spray.

FIG. 7 depicts mean QOLIE scores from a long-term safety study of diazepam nasal spray for those subjects who completed the study.

FIG. 8 depicts mean QOLIE scores from a self-administering diazepam nasal spray study comparing subjects who self-administered to non-self-administering subjects.

FIG. 9 depicts QOLIE scores from the self-administering diazepam nasal spray study only for the “seizure worry” metric.

FIG. 10 depicts QOLIE scores from the self-administering diazepam nasal spray study only for the “social functioning” metric.

FIG. 11 depicts the results of a time to second dose study of patients with seizure clusters treated with diazepam nasal spray by 6-11 years and 12-65 years age subgroups.

FIG. 12 depicts model predicated diazepam concentration curves, showing that >90% of the observed data fell within the range of the 5^{th} and 95^{th} percentiles of the predicted data.

FIG. 13 depicts numbers of who completed and did not complete a phase 3, long-term, open-label, repeat-dose, safety study of diazepam nasal spray for acute treatment of seizure clusters, as well as the most common reasons for discontinuation of the study.

FIG. 14 depicts data from a phase 3, open-label, repeat-dose safety study of diazepam nasal spray that were analyzed by subgroups receiving chronic clobazam or other intermittent and chronic benzodiazepines.

FIG. 15 depicts the mean results of QOLIE-31-P scores for quality of life and treatment satisfaction in a long-term safety study of diazepam nasal spray for seizure clusters as assessed by patients and their caregivers.

FIG. 16 depicts the mean results of QOLIE-48 scores for quality of life and treatment satisfaction in a long-term safety study of diazepam nasal spray for seizure clusters as assessed by patients and their caregivers.

FIG. 17 depicts overall QOLIE-31-P scores for a quality of life in epilepsy scale for frequent and infrequent users of diazepam nasal spray for seizure clusters.

FIG. 18 depicts “seizure worry” QOLIE-31-P scores for a quality of life in epilepsy scale for frequent and infrequent users of diazepam nasal spray for seizure clusters.

FIG. 19 depicts “social function” QOLIE-31-P scores for a quality of life in epilepsy scale for frequent and infrequent users of diazepam nasal spray for seizure clusters.

FIG. 20 depicts plasma concentration curves of both multiple dose and single dose formulations of diazepam nasal spray.

FIG. 21 depicts a graph showing the age groupings and ISCI durations of subjects that had at least two seizure clusters.

FIG. 22 depicts a graph showing the ISCI durations of groups of subjects that self-administered a benzodiazepine nasal formulation and those that did not self-administer.

FIG. 23 depicts a graph showing the ISCI durations of those subjects who were active in the study for less than 12 months and those subjects who were active in the study for at least 12 months.

FIG. 24 depicts a graph showing changes in ISCI durations from the first three months of the study to the last three months of the study.

FIG. 25 depicts a graph showing the ISCI durations of groups of subjects who had concomitant medication changes and those subjects who did not have concomitant medication changes over the course of the study.

FIG. 26A and FIG. 26B depict graphs showing the mean (FIG. 26A) and median (FIG. 26B) of a multiple period sensitivity analysis on changes in ISCI duration over time.

FIG. 27A, FIG. 27B, and FIG. 27C depicts graphs showing the mean changes is ISCI duration over time with multiple period sensitivity analysis with consistent cohorts and elimination of retreatment within 24 hours for Periods 1-4 (FIG. 27A), Periods 1-5 (FIG. 27B), and Periods 1-6 (FIG. 27C).

FIG. 28A and FIG. 28B depict graphs showing the mean (FIG. 28A) and median (FIG. 28B) of a multiple period sensitivity analysis on changes in ISCI duration over time with two groups of subjects: those that needed a single dose of the benzodiazepine nasal formulation to treat their seizures and those that needed two doses.

### DETAILED DESCRIPTION

Before the present compositions and methods are described, it is to be understood that various aspects of intranasal benzodiazepine compositions and methods of their use are disclosed herein. Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure. Unless indicated otherwise, all technical and scientific terms used herein have the meaning commonly understood by one of ordinary skill in the art. It is also to be understood that the terminology used in the description is for the purpose of describing the particular aspects or embodiments only and is not intended to limit the scope. The disclosed intranasal compositions, method of their manufacture, and methods of their use are not strictly limited to the particular compositions, processes, or methods described, as these can vary to an extend one of skill in the art will recognize without diverging from the benefits and advantages imparted by the compositions and methods. Though one of skill in the art will readily recognize obvious variations and substitutions that may be made to accomplish the same result through equivalent means or function, for the purpose of describing the various aspects and embodiments of intranasal benzodiazepine compositions, methods of their manufacture, and methods of their use, preferred compositions and methods are now described.

The compositions disclosed herein are suitable for administration to the nasal cavity. As such, the phrases “intranasal solution,” “intranasal composition,” and “intranasal formulation” are used interchangeably to mean a composition suitable for administration to the nasal mucosal membranes which line the nasal cavity.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, and so on. As a non-limiting example, each range discussed herein can be readily broken down into a lower third, middle third and upper third, and so on. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

The term “and/or” as used herein means that the listed items are present, or used, individually or in combination. In effect, this term means that “at least one of” or “one or more” of the listed items is used or present.

Surprisingly, administration of a benzodiazepine to the nasal mucosal membranes of a subject via the various intranasal compositions, as disclosed herein, induces a therapeutic benefit to the subject substantially earlier than would be expected based on measured systemic levels of the benzodiazepine. A therapeutic effect is not only experienced by the subject, but measured via EEG, well before (e.g., less than 2 minutes after administration) the systemic concentration of benzodiazepine reaches therapeutically relevant levels. As used herein, the term “pharmacodynamic” or “PD” is used to describe qualitative effects the administered benzodiazepine has on the subject, such as a change in EEG data, a change in seizure length or severity, a change in symptoms associated therewith, or a change associated with adverse effects caused by the administered benzodiazepine. The term “pharmacokinetic” or “PK” is used to describe, quantitatively, movement and processing of the benzodiazepine drug by the subject's body, such as plasma concentrations of the drug and any metabolites thereof (e.g., Cₘₐₓ, Tₘₐₓ), bioavailability, half-life, and the like. While various aspects of the PK profile of a benzodiazepine administered intranasally via the intranasal compositions as disclosed herein are similar to aspects of the PK profile of a benzodiazepine administered intravenously (e.g., similar AUC and bioavailability) and orally (e.g., similar Cₘₐₓ and Tₘₐₓ), many of the adverse effects associated with IV, oral, and rectal administration of benzodiazepines, such as somnolence, headaches, and depression/suicidal thoughts and behaviors, are reduced. Therefore, the intranasal compositions and methods of their use as provided herein represent a substantial improvement in the treatment of seizures and seizure disorders, both in the rapid realization of therapeutic benefit after administration and in improved patient compliance after experiencing reduced adverse effects.

### Compositions

As such, in one aspect, the present disclosure provides a composition suitable for intranasal administration (“intranasal composition”) comprising: a therapeutically effective amount of a benzodiazepine drug; about 0.01% w/v to about 1% w/v of an alkyl maltoside; and a carrier system comprising about 30% w/v to about 90% w/v of a natural or synthetic tocopherol, a natural or synthetic tocotrienol, or a combination thereof and about 10% w/v to about 70% w/v of one or more alcohols.

Benzodiazepines have the general basic structure of formula I:

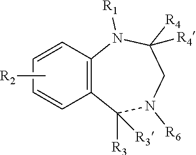

wherein R₁-R₅ are substitutable chemical moieties. R₁ may be an optionally substituted alkyl or may form a optionally substituted heterocyclic ring with R₄ (where the hetero atom is the nitrogen (N) in the diazepine ring); R₂ is a halogen (e.g., Cl, Br); R₃ may be an optionally substituted aryl group (e.g., 2-chloro or 2-fluorophenyl); R₅ is —H or —OH; if R₄ is not joined with R₁ to form an optionally substituted heterocyclic ring, R₄ and R₄′ may together form a carbonyl moiety (C═O) with the carbon to which they are attached; R₃′ and R₆ may together form a double bond or may be combined to form an optionally substituted heterocyclic ring fused to the diazepine ring at the atoms to which they are attached. Benzodiazepines are basic compounds, and as such, may form acid addition salts with pharmaceutically acceptable acids, such as pharmaceutically acceptable mineral acids and pharmaceutically acceptable organic acids. Reference to a benzodiazepine herein refers to and includes any pharmaceutically acceptable form, such as the free base form, an acid addition salt, a base addition salt, or a solvated form (such as a hydrate).

Pharmaceutically acceptable mineral acids include hydrochloric acid, sulfuric acid, sulfurous acid, phosphoric acid, phosphorous acid, and others that will be recognized by those of skill in the art. Pharmaceutically acceptable organic acids include acetic acid, benzoic acid, tartaric acid, citric acid, oxalic acid, maleic acid, malonic acid, 1-hydroxy-2-naphthoic acid, 2,2-dichloroacetic acid, 2-hydroxyethanesulfonic acid, 2-oxoglutaric acid, 4-acetaidobenzoic acid, 4-ainosalicylic acid, acetic acid, adipic acid, ascorbic acid (L), aspartic acid (L), benzenesulfonic acid, benzoic acid, camphoric acid (+), caphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (-L), malonic acid, mandelic acid (DL), methanesulfonic acid, benzenesulfonic acid (besylic acid), naphthalene-1,5-disulfonic acid, naphthalene-2-sulfonic acid, nicotinic acid, nitric acid, oleic acid, oxalic acid, palmitic acid, pamoic acid, phosphoric acid, propionic acid, pyroglutamic acid (-L), salicylic acid, sebacic acid, stearic acid, succinic acid, sulfuric acid, tartaric acid (+L), thiocyanic acid, toluenesulfonic acid (p), and undecylenic acid. Other pharmaceutically acceptable acids may be pharmaceutically acceptable acidic (anionic) polymers or pharmaceutically acceptable amphoteric polymers. One skilled in the art will recognize that other basic active pharmaceutical ingredients may be combined with the foregoing acids to produce acid addition salts.

Examples of benzodiazepines that may be delivered intranasally via the intranasal compositions as disclosed herein include, but are not limited to, alprazolam, brotizolam, chlordiazepoxide, clobazam, clonazepam, clorazepam, demoxazepam, diazepam, flumazenil, flurazepam, halazepam, olanzapine, midazolam, nordazepam, medazepam, nitrazepam, oxazepam, lorazepam, prazepam, quazepam, triazolam, temazepam, loprazolam, any pharmaceutically acceptable salt thereof, as well as any combinations thereof. For example, particularly useful compositions may comprise diazepam, midazolam, lorazepam, alprazolam, or a pharmaceutically acceptable salt thereof.

Compositions for delivering a benzodiazepine intranasally (“intranasal compositions”) comprise a therapeutically effective amount of a benzodiazepine, for example, about 1 mg to about 20 mg of the benzodiazepine per a volume of about 10 μL to 200 μL. For example, an intranasal composition may comprise about 5 mg/mL (0.5% w/v) to about 600 mg/mL (60% w/v) or about 10 mg/mL to about 250 mg/mL of a benzodiazepine, which also includes concentrations of about 1% w/v to about 50% w/v, about 5% w/v to about 25% w/v, about 5% w/v to about 15% w/v, or about 5% w/v to about 20% w/v of a benzodiazepine. These ranges include any discreet concentrations within the disclosed ranges, such as, about 5% w/v, about 7.5% w/v, about 10% w/v, about 15% w/v, and about 20% w/v of a benzodiazepine.

The intranasal compositions as disclosed herein comprise a benzodiazepine dissolved in a carrier system comprising a natural or synthetic tocopherol, a natural or synthetic tocotrienol, or a combination thereof and one or more alcohols.

The intranasal compositions may comprise about 30% w/v to about 90% w/v of a natural or synthetic tocopherol, a natural or synthetic tocotrienol, or a combination thereof, such as about 50% w/v to about 75% w/v, about 50% w/v to about 60% w/v, about 45% w/v to about 65% w/v, about 45% w/v to about 85% w/v, about 10% w/v to about 25% w/v, about 25% w/v to about 65% w/v, about 35% w/v to about 55% w/v, about 35% w/v to about 45% w/v, or about 40% w/v to about 42% w/v of a natural or synthetic tocopherol, a natural or synthetic tocotrienol, or a combination thereof.

Examples of suitable natural or synthetic tocopherols or tocotrienols include, but are not limited to, α-tocopherol, β-tocopherol, γ-tocopherol, δ-tocopherol, α-tocotrienol, β-tocotrienol, γ-tocotrienol, δ-tocotrienol, tocophersolan, an isomer of any thereof, an ester of any thereof, an analog or derivative of any thereof, and any combination thereof. A synthetic tocopherol may be covalently bonded to a glycol polymer, such as polyethylene glycol, as in vitamin E TPGS (vitamin E polyethylene glycol succinate). Alternatively, the intranasal compositions as disclosed herein may be free of, or substantially free of, such glycol-bound synthetic tocopherols. Many of the various tocopherol and tocotrienols solvents described above are naturally-occurring vitamin E compounds or vitamin E esters. Vitamin E is a class of fat-soluble methylated phenols. As used herein, vitamin E refers to any of the natural or synthetic tocopherols, tocotrienols, isomers thereof, esters thereof, or any analogs or derivatives thereof, as well as combinations thereof. It has been found that vitamin E is an effective carrier for benzodiazepines and does not irritate sensitive mucosal membranes. Typically, vitamin E is considered hydrophobic and, as such, is used in aqueous-based emulsion-type compositions which tend to be physically unstable. However, when including a vitamin E carrier with one or more lower alcohols as a true solution, a composition may be provided having enhanced stability and suitability as a carrier for intranasal administration of a benzodiazepine. In some embodiments, the intranasal compositions may comprise about 35% w/v to about 45% w/v of vitamin E. In some embodiments, the intranasal compositions may comprise about 40% w/v to about 42% w/v of vitamin E.

Therefore, the carrier system of the intranasal compositions disclosed herein also comprises about 10% w/v to about 70% w/v of one or more alcohols. As used herein, “alcohol” is used to describe a molecule having at least one hydroxyl functional group (—OH) bound to a saturated carbon atom, which includes monohydric alcohols and polyhydric alcohols, such as glycols. The alcohol may be a lower alcohol, which includes compounds with six or fewer carbon atoms, such as ethanol, propanol, butanol, pentanol, benzyl alcohol, ethylene glycol, propylene glycol, butylene glycol, pentylene glycol, any isomer thereof, or any combination thereof. An intranasal composition may comprise about 10% w/v to about 70% w/v of one or more alcohols such as about 15% w/v to about 55% w/v, about 25% w/v to about 45% w/v, about 35% w/v to about 45% w/v, or about 30% w/v of one or more alcohols. For example, an intranasal composition may comprise 15% w/v to about 55% w/v, or about 25% w/v to about 40% w/v, or about 35% w/v to about 45% w/v, or about 30% w/v of benzyl alcohol, ethanol, or a mixture thereof. In another example, an intranasal composition may comprise a mixture of ethanol and benzyl alcohol. In any embodiment, an intranasal composition may comprise about 10% w/v to about 25% w/v of ethanol and about 5% w/v to about 15% w/v of benzyl alcohol, or about 15% w/v to about 22.5% w/v ethanol and about 7.5% w/v to about 12.5% w/v benzyl alcohol, or about 10% w/v to about 25% w/v ethanol and about 7.5% w/v to about 12.5% w/v benzyl alcohol, or about 17% w/v to about 20% w/v ethanol and about 10% w/v to about 12% w/v benzyl alcohol. In any embodiment, an intranasal composition may comprise about 5% w/v to about 15% w/v of ethanol and about 10% w/v to about 25% w/v of benzyl alcohol, or about 7.5% w/v to about 12.5% w/v ethanol and about 15% w/v to about 22.5% w/v benzyl alcohol, or about 7.5% w/v to about 12.5% w/v ethanol and about 10% w/v to about 25% w/v benzyl alcohol, or about 10% w/v to about 12% w/v ethanol and about 17% w/v to about 20% w/v benzyl alcohol. In some embodiments, an intranasal composition may comprise 15% w/v to about 55% w/v of benzyl alcohol. In some embodiments, an intranasal composition may comprise about 35% w/v to about 45% w/v of benzyl alcohol. In some embodiments, an intranasal composition may comprise about 40% w/v to about 42% w/v of benzyl alcohol.

Optionally and in any embodiment, an intranasal composition, as disclosed herein, may be substantially free or free of polymeric glycols, such as polyethylene glycol, without diminishing the therapeutic benefit of the benzodiazepine administered via the intranasal composition. For example, in any embodiment, an intranasal composition, as disclosed herein, may be substantially free or free of a polymeric glycol having a molecular weight greater than about 200 g/mol. Additionally or alternatively, in any embodiment, an intranasal composition as disclosed herein may comprise very little water, substantially no water, or are completely free of water and are non-aqueous. For example, an intranasal composition may consist essentially of or consist of 1) a benzodiazepine drug; 2) one or more alkyl maltosides (e.g., DDM and/or TDM), and 3) a carrier system consisting of a) one or more natural or synthetic tocopherols or tocotrienols and b) one or more alcohols, and is optionally substantially free of water.

In addition to the benzodiazepine and the carrier system described above, the intranasal compositions, as disclosed herein, comprise about 0.01% w/v to about 1% w/v of an alkyl maltoside, such as octyl-, nonyl-, decyl-, undecyl-, dodecyl, tridecyl, tetradecyl, pentadecyl, or octadecyl α- or β-D-maltoside. In any embodiment, an intranasal composition may comprise one or both of dodecyl maltoside (n-dodecyl β-D-maltoside or DDM) and tetradecyl maltoside (TDM). For example, in any embodiment, an intranasal composition may comprise about 0.01% (w/v) to about 1% (w/v) of the alkyl maltoside, such as about 0.05% (w/v) to about 0.75% (w/v), 0.05% (w/v) to about 0.5% (w/v), 0.125% (w/v) to about 0.75% (w/v), or about 0.125% (w/v) to about 0.5% (w/v). In particular examples, an intranasal composition comprises about 0.1% w/v to about 0.75% w/v dodecyl maltoside (DDM), about 0.1% w/v to about 0.5% w/v DDM, about 0.15% w/v to about 0.3% w/v DDM, about 0.15% w/v to about 0.5% w/v DDM, about 0.18% w/v DDM, about 0.25% w/v DDM, about 0.5% w/v DDM, or about 0.75% w/v DDM.

Advantageously, it has been observed that the intranasal compositions, as described herein, do not support the growth of bacteria and therefore may be substantially free or free of any antibacterial agents or other preservatives. However, the use of an antibacterial does not preclude the therapeutic benefits of administering a benzodiazepine via an intranasal composition as described herein. Therefore, in any intranasal composition as disclosed herein, one or more additional preservation, anti-degradation, antibacterial, or antifungal agents may be included. An intranasal composition, as disclosed herein, may further optionally comprise one or more agents to enhance appearance, taste, or odor.

In some embodiments, any of the intranasal compositions or formulations provided herein are for a single dose nasal administration.

In some embodiments, a single dose intranasal composition may comprise diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.25% (w/v). In some embodiments, a single dose intranasal composition may comprise diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.50% (w/v). In some embodiments, a single dose intranasal composition may comprise diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.75% (w/v).

In some embodiments, a single dose intranasal composition may comprise diazepam or a pharmaceutically acceptable salt thereof in an amount of about 20% (w/v); vitamin E in an amount from about 40% to about 42% (w/v); benzyl alcohol in an amount from about 40% to about 42% (w/v); and n-dodecyl beta D-maltoside (DDM) in an amount of about 0.25% (w/v). In some embodiments, a single dose intranasal composition may comprise diazepam or a pharmaceutically acceptable salt thereof in an amount of about 20% (w/v); vitamin E in an amount from about 40% to about 42% (w/v); benzyl alcohol in an amount from about 40% to about 42% (w/v); and n-dodecyl beta D-maltoside (DDM) in an amount of about 0.50% (w/v). In some embodiments, a single dose intranasal composition may comprise diazepam or a pharmaceutically acceptable salt thereof in an amount of about 20% (w/v); vitamin E in an amount from about 40% to about 42% (w/v); benzyl alcohol in an amount from about 40% to about 42% (w/v); and n-dodecyl beta D-maltoside (DDM) in an amount of about 0.75% (w/v).

In some embodiments, a single dose intranasal composition may comprise diazepam or a pharmaceutically acceptable salt thereof in an amount of about 20% (w/v); vitamin E in an amount from about 41.86% (w/v); benzyl alcohol in an amount from about 41.50% (w/v); and n-dodecyl beta D-maltoside (DDM) in an amount of about 0.25% (w/v). In some embodiments, a single dose intranasal composition may comprise diazepam or a pharmaceutically acceptable salt thereof in an amount of about 20% (w/v); vitamin E in an amount from about 41.68% (w/v); benzyl alcohol in an amount from about 41.50% (w/v); and n-dodecyl beta D-maltoside (DDM) in an amount of about 0.50% (w/v). In some embodiments, a single dose intranasal composition may comprise diazepam or a pharmaceutically acceptable salt thereof in an amount of about 20% (w/v); vitamin E in an amount from about 41.50% (w/v); benzyl alcohol in an amount from about 41.50% (w/v); and n-dodecyl beta D-maltoside (DDM) in an amount of about 0.75% (w/v).

### Methods of Use

The intranasal compositions disclosed herein provide therapeutic benefit to a subject when administered to the intranasal mucosal membrane of the subject. For example, the intranasal compositions comprising a benzodiazepine drug; about 0.01% w/v to about 1% w/v of an alkyl maltoside; and a carrier system comprising about 30% w/v to about 90% w/v of a natural or synthetic tocopherol, a natural or synthetic tocotrienol, or a combination thereof, and about 10% w/v to about 70% w/v of one or more alcohols, as disclosed herein, provide therapeutic benefit to a subject when administered to the intranasal mucosal membrane of the subject. Administration of the intranasal composition may comprise spraying at least a portion of a therapeutically effective amount of a benzodiazepine via the intranasal composition into at least one nostril, such that the intranasal composition contacts the nasal mucosal membrane of the subject. For example, administration of the intranasal composition may comprise spraying a single dose of a therapeutically effective amount of a benzodiazepine via the intranasal composition into at least one nostril, such that the intranasal composition contacts the nasal mucosal membrane of the subject. In another example, administration of the intranasal composition may comprise spraying a single dose of a therapeutically effective amount of a benzodiazepine via the intranasal composition into at least one nostril, and optionally, after a time delay, spraying a second single dose of the intranasal composition into one nostril. In another example, administration of the intranasal composition may comprise spraying at least a portion of a therapeutically effective amount of a benzodiazepine via the intranasal composition into each nostril. In yet another example, administration of the intranasal composition may comprise spraying a first quantity of the intranasal composition into the first nostril, spraying a second quantity of the intranasal composition into a second nostril, and optionally after a pre-selected time delay, spraying a third quantity of the intranasal composition into the first nostril. Optionally after a pre-selected time delay, a fourth quantity of the intranasal composition may be administered to the second nostril.

A benzodiazepine may be administered via any intranasal composition as disclosed herein to treat a condition, disorder, syndrome, or disease for which administration of a benzodiazepine drug may provide therapeutic benefit. Non-limiting examples of benzodiazepines that may be administered via the intranasal compositions, as disclosed herein, for therapeutic benefit include alprazolam, brotizolam, chlordiazepoxide, clobazam, clonazepam, clorazepam, demoxazepam, diazepam, flumazenil, flurazepam, halazepam, olanzapine, midazolam, nordazepam, medazepam, nitrazepam, oxazepam, lorazepam, prazepam, quazepam, triazolam, temazepam, loprazolam, any pharmaceutically acceptable salt thereof, as well as any combinations thereof. The condition, disorder, syndrome, or disease may have associated therewith undesirable symptoms which may be ameliorated through the administration of a benzodiazepine. In one example, the conditions, disorder, syndrome, disease, or symptoms thereof may be treated by administering the intranasal composition at any time before or after onset of a symptom of the condition, disorder, syndrome, or disease which may be treatable with a benzodiazepine. For example, the intranasal compositions disclosed herein, comprising a benzodiazepine, may be used to treat conditions, disorders, syndromes, and diseases as well as symptoms associated therewith, such as seizures and seizure disorders, such as epilepsy.

The intranasal compositions, as disclosed herein, may be used in preventing (i.e., inhibiting the onset of a seizure) or treating a seizure or seizure disorder, condition, syndrome, or disease. Therefore, in one aspect, the present disclosure provides a method of preventing or treating a seizure in a subject in need thereof comprising administering a composition comprising: an effective amount of a benzodiazepine (such as diazepam, lorazepam, midazolam, or the like); about 0.01% w/v to about 1% w/v of an alkyl maltoside; and a carrier system comprising about 30% w/v to about 90% w/v of a natural or synthetic tocopherol, a natural or synthetic tocotrienol, or a combination thereof, and about 10% w/v to about 70% w/v of one or more alcohols, to a nasal mucosal membrane of the subject. In some embodiments, an intranasal composition, as disclosed herein, may comprise about 5 mg to about 20 mg of diazepam, about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, an intranasal composition, as disclosed herein, may comprise about 5 mg to about 20 mg of diazepam, about 0.01% w/v to about 1% w/v of an alkyl maltoside, about 40% w/v to about 70% w/v of a natural or synthetic tocopherol or tocotrienol, and about 5% w/v to about 45% w/v of one or more alcohols (e.g., benzyl alcohol). In some embodiments, a method of inhibiting the onset of a seizure or treating a seizure in a subject in need thereof may comprise administering to the subject a single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.25% (w/v). In some embodiments, a method of inhibiting the onset of a seizure or treating a seizure in a subject in need thereof may comprise administering to the subject a single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.50% (w/v). In some embodiments, a method of inhibiting the onset of a seizure or treating a seizure in a subject in need thereof may comprise administering to the subject a single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.75% (w/v). Any intranasal composition, as disclosed herein, is suitable and may be used to treat or prevent seizures, such as epileptic seizures (including refractory epilepsy), absence seizures, myoclonic seizures, clonic seizures, tonic seizures, tonic-clonic seizures, atonic seizures, focal seizures, atonic seizures, and combinations thereof. Seizures that may be treated with the disclosed intranasal compositions include acute seizures, acute repetitive seizures (e.g., a seizure that is part of a seizure cluster).

The progression of a seizure event may be divided into phases. For example, a seizure event may comprise one or more of a prodromal phase, a pre-ictal phase, an ictal phase/peri-ictal phase, and a post-ictal phase. In accordance with the methods of treating or inhibiting the onset of a seizure as disclosed herein, a benzodiazepine may be administered via an intranasal composition as disclosed herein during any phase, such as the prodromal, pre-ictal, and ictal/peri-ictal, post-ictal, or interictal phase. In some embodiments, a benzodiazepine is administered via an intranasal composition as disclosed herein during a prodromal or pre-ictal phase prevent or reduce the severity or length of an impending seizure. In some embodiments, a benzodiazepine is administered via an intranasal composition as disclosed herein during the pre-ictal or ictal phase to acutely treat an active seizure. In some embodiments, for example, if the intranasal composition is unavailable during a prodromal, pre-ictal, or ictal phase, the intranasal composition may be administered during a post-ictal phase. Such an administration may serve, for example, to prevent a subsequent seizure or to reduce any lingering effects from the current seizure event.

A subject may or may not recognize a prodromal phase as a feeling or sensation, such as confusion, anxiety, irritability, headache, tremor, anger, or other mood disturbance. The prodromal phase is not generally characterized as part of an active seizure but may serve as a warning sign to the subject of an impending seizure. Thus, in any embodiment, the intranasal compositions disclosed herein may be administered during the prodromal phase to prevent a seizure from occurring or reduce the severity of the seizure. In the absence of or in addition to a subject's self-recognition of a prodromal phase, a subject may employ a monitoring device, for example, a medical device such as those available from EMPATICA™, that measures various biometric data of the subject to enable identification of an impending seizure and therefore suggest administration of an intranasal composition, as disclosed herein, prior to the start of a seizure. Therefore, in another aspect, the present disclosure provides a method of preventing a seizure, inhibiting the onset of a seizure, or reducing the severity or length of an impending seizure in a subject comprising administering a benzodiazepine during a prodromal phase via an intranasal composition comprising an effective amount of a diazepam; about 0.01% w/v to about 1% w/v of an alkyl maltoside; and a carrier system comprising about 30% w/v to about 90% w/v of a natural or synthetic tocopherol, a natural or synthetic tocotrienol, or a combination thereof, and about 10% w/v to about 70% w/v of one or more alcohols to the nasal mucosal membrane of the subject. In some embodiments, a method of preventing a seizure, inhibiting the onset of a seizure, or reducing the severity or length of an impending seizure in a subject comprises administering a benzodiazepine during a prodromal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.25% (w/v). In some embodiments, a method of preventing a seizure, inhibiting the onset of a seizure, or reducing the severity or length of an impending seizure in a subject comprises administering a benzodiazepine during a prodromal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.50% (w/v). In some embodiments, a method of preventing a seizure, inhibiting the onset of a seizure, or reducing the severity or length of an impending seizure in a subject comprises administering a benzodiazepine during a prodromal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.75% (w/v).

Pre-ictal (or early ictal) likewise may not be experienced by every subject but may include experience of aura or sensory disturbance. Examples include, but are not limited to, vision loss/blurring, flickering vision, hallucinations, ringing/buzzing sounds, strange smells, bitter/acidic taste, out-of-body sensation, nausea, numbness, tingling, dizziness, pain, twitching, strong emotions, déjà vu, or jamais vu. Therefore, in another aspect, the present disclosure provides a method of preventing or reducing the severity or length of an impending seizure in a subject comprising administering, for example, by self-administration, a benzodiazepine during a pre-ictal phase via an intranasal composition comprising an effective amount of a diazepam; about 0.01% w/v to about 1% w/v of an alkyl maltoside; and a carrier system comprising about 30% w/v to about 90% w/v of a natural or synthetic tocopherol, a natural or synthetic tocotrienol, or a combination thereof, and about 10% w/v to about 70% w/v of one or more alcohols to the nasal mucosal membrane of the subject. In some embodiments, a method of preventing a seizure, inhibiting the onset of a seizure, or reducing the severity or length of an impending seizure in a subject comprises administering a benzodiazepine during a pre-ictal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.25% (w/v). In some embodiments, a method of preventing a seizure, inhibiting the onset of a seizure, or reducing the severity or length of an impending seizure in a subject comprises administering a benzodiazepine during a pre-ictal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.50% (w/v). In some embodiments, a method of preventing a seizure, inhibiting the onset of a seizure, or reducing the severity or length of an impending seizure in a subject comprises administering a benzodiazepine during a pre-ictal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.75% (w/v).

An ictal/peri-ictal phase includes the time period wherein active seizure is experienced or physiologically measured and may include symptoms such as, but not limited to, confusion, memory lapse, distractedness, sense of detachment, eye or head twitching movement in one direction, inability to move or speak, loss of bladder and/or bowel control, pale/flushed skin, hearing loss, strange sounds, vision loss, blurring, flashing vision, chewing or lip-smacking, unusual physical activity, walking/running, pupil dilation, difficulty breathing, racing heart, sweating, tremors, twitching, arm or leg stiffening, numbness, or drooling. Therefore, in another aspect, the present disclosure provides a method of reducing the severity or length of an active seizure in a subject comprising administering a benzodiazepine during an ictal or peri-ictal phase via an intranasal composition comprising an effective amount of a diazepam; about 0.01% w/v to about 1% w/v of an alkyl maltoside; and a carrier system comprising about 30% w/v to about 90% w/v of a natural or synthetic tocopherol, a natural or synthetic tocotrienol, or a combination thereof, and about 10% w/v to about 70% w/v of one or more alcohols to the nasal mucosal membrane of the subject. In some embodiments, a method of preventing a seizure, inhibiting the onset of a seizure, or reducing the severity or length of an impending seizure in a subject comprises administering a benzodiazepine during an ictal or peri-ictal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.25% (w/v). In some embodiments, a method of preventing a seizure, inhibiting the onset of a seizure, or reducing the severity or length of an impending seizure in a subject comprises administering a benzodiazepine during an ictal or peri-ictal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.50% (w/v). In some embodiments, a method of preventing a seizure, inhibiting the onset of a seizure, or reducing the severity or length of an impending seizure in a subject comprises administering a benzodiazepine during an ictal or peri-ictal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.75% (w/v).

After the seizure or ictal phase ends, a subject may experience a recovery or post-ictal phase which may span a minute or less or may continue for minutes, hours, or days. Typical symptoms of a post-ictal phase include, but are not limited to, drowsiness, confusion, memory loss, nausea, general malaise, body soreness, difficulty finding names/words, headaches, thirst, arm/leg weakness, hypertension, or feelings of fear, embarrassment, or sadness. The period between the post-ictal phase and the beginning of the next seizure is termed the “interictal” phase. Therefore, in another aspect, the present disclosure provides a method of preventing or reducing the severity or length of a subsequent seizure in a subject comprising administering a benzodiazepine during a post-ictal or interictal phase via an intranasal composition comprising an effective amount of a diazepam; about 0.01% w/v to about 1% w/v of an alkyl maltoside; and a carrier system comprising about 30% w/v to about 90% w/v of a natural or synthetic tocopherol, a natural or synthetic tocotrienol, or a combination thereof, and about 10% w/v to about 70% w/v of one or more alcohols to the nasal mucosal membrane of the subject. In some embodiments, a method of preventing a seizure, inhibiting the onset of a seizure, or reducing the severity or length of an impending seizure in a subject comprises administering a benzodiazepine during a post-ictal or interictal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.25% (w/v). In some embodiments, a method of preventing a seizure, inhibiting the onset of a seizure, or reducing the severity or length of an impending seizure in a subject comprises administering a benzodiazepine during a post-ictal or interictal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.50% (w/v). In some embodiments, a method of preventing a seizure, inhibiting the onset of a seizure, or reducing the severity or length of an impending seizure in a subject comprises administering a benzodiazepine during a post-ictal or interictal phase via an single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.75% (w/v).

In any embodiment, an intranasal composition can comprise an effective amount of diazepam. Effective amounts of diazepam that may be administered via the intranasal compositions, as disclosed herein, include about 5 mg to about 20 mg of diazepam, such as about 5 mg to about 15 mg of diazepam, about 5 mg to about 10 mg of diazepam, about 10 mg to about 20 mg of diazepam, or about 15 mg to about 20 mg. Effective dosing may, in any embodiment, be determined based on the body weight of the subject to which the diazepam will be administered. For example, for any given subject having a body weight (in kg), a dose of about 0.25 mg/kg to about 0.60 mg/kg may be administered. For example, suitable doses include about 0.27 mg/kg, about 0.35 mg/kg, about 0.40 mg/kg, about 0.50 mg/kg, or about 0.55 mg/kg of body weight may be administered to a subject. Diazepam may be included in a composition comprising an about 0.01% w/v to about 1% w/v of an alkyl maltoside and a carrier system comprising about 30% w/v to about 90% w/v of a natural or synthetic tocopherol, a natural or synthetic tocotrienol, or a combination thereof, and about 10% w/v to about 70% w/v, the intranasal composition having a volume of about 10 μL to about 200 μL, about 50 μL to about 150 μL, about 75 μL to about 125 μL, about 75 μL, about 100 μL, or about 125 μL. For example, 5 mg, 7.5 mg, 10 mg, 15 mg, or 20 mg diazepam may be administered in a volume of about 75 μL, 100 μL, or 125 μL. A dose may be administered to a single nostril or split up between nostrils. For example, a dose of 5 mg diazepam in 100 μL, 7.5 mg diazepam in 100 μL, 10 mg diazepam in 100 μL, or 20 mg diazepam in 100 μL may be administered in a single nostril. A dose of 10 mg may alternatively be administered as a dose of 5 mg diazepam in 100 μL in each nostril. Similarly, a 15 mg dose may be administered, for example, as 7.5 mg diazepam in 100 μL to each nostril. A 20 mg dose may be administered, for example, 10 mg diazepam in 100 μL to each nostril.

In one embodiment, the intranasal composition administered in accordance with any of the methods disclosed herein comprises 5% w/v benzodiazepine, 56.5% w/v vitamin E, 0.25% w/v DDM, 10.5% w/v benzyl alcohol, and an amount of ethanol sufficient to reach the desired volume (e.g., 100 μL).

In one embodiment, the intranasal composition administered in accordance with any of the methods disclosed herein comprises 7.5% w/v benzodiazepine, 56.5% w/v vitamin E, 0.25% w/v DDM, 10.5% w/v benzyl alcohol, and an amount of ethanol sufficient to reach the desired volume (e.g., 100 μL).

In one embodiment, the intranasal composition administered in accordance with any of the methods disclosed herein comprises 10% w/v benzodiazepine, 56.5% w/v vitamin E, 0.25% w/v DDM, 10.5% w/v benzyl alcohol, and an amount of ethanol sufficient to reach the desired volume (e.g., 100 μL).

In some embodiments, the single dose intranasal composition administered in accordance with any of the methods disclosed herein comprises about 20% w/v benzodiazepine, about 35% to about 45% w/v vitamin E, about 35% to about 45% w/v benzyl alcohol, and about 0.25% DDM in a desired volume (e.g., 100 μL). In some embodiments, the single dose intranasal composition administered in accordance with any of the methods disclosed herein comprises about 20% w/v benzodiazepine, about 40% to about 42% w/v vitamin E, about 40% to about 42% w/v benzyl alcohol, and about 0.25% DDM in a desired volume (e.g., 100 μL).

In one embodiment, the single dose intranasal composition administered in accordance with any of the methods disclosed herein comprises about 20% w/v benzodiazepine, about 35% to about 45% w/v vitamin E, about 35% to about 45% w/v benzyl alcohol, and about 0.50% DDM in a desired volume (e.g., 100 μL). In one embodiment, the single dose intranasal composition administered in accordance with any of the methods disclosed herein comprises about 20% w/v benzodiazepine, about 40% to about 42% w/v vitamin E, about 40% to about 42% w/v benzyl alcohol, and about 0.50% DDM in a desired volume (e.g., 100 μL).

In one embodiment, the single dose intranasal composition administered in accordance with any of the methods disclosed herein comprises about 20% w/v benzodiazepine, about 35% to about 45% w/v vitamin E, about 35% to about 45% w/v benzyl alcohol, and about 0.75% DDM in a desired volume (e.g., 100 μL). In one embodiment, the single dose intranasal composition administered in accordance with any of the methods disclosed herein comprises about 20% w/v benzodiazepine, about 40% to about 42% w/v vitamin E, about 40% to about 42% w/v benzyl alcohol, and about 0.75% DDM in a desired volume (e.g., 100 μL).

An effective amount of a benzodiazepine may be administered to a subject via an intranasal composition, as disclosed herein, multiple times, if necessary, to effectively treat or prevent a seizure. In accordance with the methods disclosed herein, the administering is carried out to reduce one or more of the frequency, length, and severity of recurrent seizures in the subject. As such, in another aspect, the present disclosure provides a method of preventing (i.e., inhibiting the onset of a seizure) or treating a seizure comprising: administering a first dose of a composition comprising an effective amount of a benzodiazepine, such as diazepam; about 0.01% w/v to about 1% w/v of an alkyl maltoside; and a carrier system comprising about 30% w/v to about 90% w/v of a natural or synthetic tocopherol, a natural or synthetic tocotrienol, or a combination thereof, and about 10% w/v to about 70% w/v of one or more alcohols to a nasal mucosal membrane of the subject during or before a seizure, wherein when adequate cessation or prevention of the seizure is not achieved within 4 hours, 2 hours, 1 hour, 30 minutes, 15 minutes, or 10 minutes after the administering of the first dose, one or more subsequent doses of the composition are administered to the subject. For example, a composition comprising about 5 mg to about 20 mg of diazepam, about 0.01% w/v to about 1% w/v of an alkyl maltoside; and a carrier system comprising about 30% w/v to about 90% w/v of a natural or synthetic tocopherol, a natural or synthetic tocotrienol, or a combination thereof and about 10% w/v to about 70% w/v of one or more alcohols may be administered in a first dose and upon inadequate cessation of the seizure, a second dose of the composition may be administered. The second dose may be identical in volume and strength as the first dose. Optionally, if adequate cessation or prevention of the seizure is not achieved within 4 hours, 2 hours, 1 hour, 30 minutes, 15 minutes, or 10 minutes after administration of the second dose, the method may further comprise administering a third dose of the composition.

Advantageously, administration of a benzodiazepine via an intranasal composition, as disclosed herein, to a subject may induce a therapeutic benefit to the subject substantially before a therapeutically relevant benzodiazepine concentration is achieved in the blood plasma of the subject. For example, maximum plasma concentrations (Cₘₐₓ) of about 200 ng/mL to about 500 ng/mL may be reached at a Tₘₐₓ of about an hour or more, such as about 1 hour, about 1.25 hours, about 1.5 hours, about 1.75 hours, or about 2 hours following administration of a benzodiazepine via an intranasal composition as described herein. As such, the PK profiles of intranasal compositions, as disclosed herein, appear similar to oral formulations (see, e.g., Friedman, et al., Clinical Pharmacology & Therapeutics, 1992; 52(2): pp. 139-150, which is incorporated in its entirety herein by reference.) Advantageously and surprisingly, the PK profiles of single dose compositions, as disclosed herein, are superior to the PK profiles of multiple dose compositions. In some embodiments, the administration of a single dose intranasal composition provided herein results in a Cmax of over 250 ng/mL in a subject. In some embodiments, the administration of a single dose intranasal composition provided herein results in a Cmax of about 258.7 ng/mL in a subject. In some embodiments, the administration of a single dose intranasal composition provided herein results in a Tₘₐₓ of under 1.5 hours in a subject. In some embodiments, the administration of a single dose intranasal composition provided herein results in a Tₘₐₓ of about 1.25 hours in a subject. Therefore, in one aspect the present disclosure provides a method of reaching a Cₘₐₓ of over 250 ng/mL, a Tₘₐₓ of under 1.5 hours, or both, of a benzodiazepine or a pharmaceutically acceptable salt thereof in a subject, the method comprising a single dose intranasal administration of a composition comprising: an effective amount of a benzodiazepine (such as diazepam, lorazepam, midazolam, or the like); about 0.01% w/v to about 1% w/v of an alkyl maltoside; and a carrier system comprising about 30% w/v to about 90% w/v of a natural or synthetic tocopherol, a natural or synthetic tocotrienol, or a combination thereof, and about 10% w/v to about 70% w/v of one or more alcohols, to a nasal mucosal membrane of the subject. In some embodiments, a method of reaching a Cₘₐₓ of over 250 ng/mL, a Tₘₐₓ of under 1.5 hours, or both, of a benzodiazepine or a pharmaceutically acceptable salt thereof in a subject comprises a single dose intranasal administration of a composition comprising: diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.25% (w/v), to a nasal mucosal membrane of the subject. In some embodiments, a method of reaching a Cₘₐₓ of over 250 ng/mL, a Tₘₐₓ of under 1.5 hours, or both, of a benzodiazepine or a pharmaceutically acceptable salt thereof in a subject comprises a single dose intranasal administration of a composition comprising: diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.50% (w/v), to a nasal mucosal membrane of the subject. In some embodiments, a method of reaching a Cₘₐₓ of over 250 ng/mL, a Tₘₐₓ of under 1.5 hours, or both, of a benzodiazepine or a pharmaceutically acceptable salt thereof in a subject comprises a single dose intranasal administration of a composition comprising: diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.75% (w/v), to a nasal mucosal membrane of the subject. In any of the above embodiments, the Cₘₐₓ of over 250 ng/mL is a Cₘₐₓ of about 258.7 ng/mL. In any of the above embodiments, the Tₘₐₓ of under 1.5 hours is a Tₘₐₓ of about 1.25 hours.

Several clinical studies with intranasal formulations of benzodiazepines (e.g., diazepam) have been reported in the literature. Generally, the absolute bioavailability of the benzodiazepine administered via these formulations was low (about 50%). For example, Gizurarson S. and Bechgaard E; Diabetes Res Clin Pract. 1991; 12(2): 71-84, which is incorporated in its entirety herein by reference, reports the administering of a 2 mg dose of a 20 mg/mL diazepam solution dissolved in 5% glycofurol in polyethylene glycol 200. Mean bioavailability of the benzodiazepine was reported to be 50.4%±23.3% with a Tₘₐₓ of 18±11 minutes. Lindhardt, et al.; Br J Clin Pharmacol. 2001; 52(5): 521-527, which is incorporated in its entirety herein by reference, evaluated an intranasal formulation of diazepam with doses of 4 mg and 7 mg in polyethylene glycol 300 in seven healthy volunteers against a 5 mg IV dose. The benzodiazepine administered via the intranasal formulation had a relative bioavailability of 45% and 42%, a Cₘₐₓ of 99 ng/mL and 170 ng/mL, and a Tₘₐₓ of 18 minutes and 42 minutes for the 4 mg and 7 mg doses, respectively. Ivaturi et al.; Acta Neurol Scand. 2009; 120(5): 353-357, which is incorporated in its entirety herein by reference, conducted a study of the bioavailability and tolerability of diazepam administered to healthy volunteers intranasally. They compared 5 mg and 10 mg intranasal diazepam doses with a 5 mg IV dose of diazepam. Following the 5 mg and 10 mg doses, the median Tₘₐₓ was 20 and 30 minutes, respectively, and the mean Cₘₐₓ was 134.3±62 ng/mL and 247.6±61 ng/mL. Estimated bioavailability of diazepam was 75% for both doses.

In contrast, absolute bioavailability of a benzodiazepine (e.g., diazepam) administered via an intranasal composition, as disclosed herein, rivals that of the same benzodiazepine administered intravenously (about 97%). In addition, the mean elimination half-life of benzodiazepine following administration of the intranasal composition described herein is significantly longer than other intranasal benzodiazepine compositions (i.e., t_{1/2} of ˜49 hours vs. 2-6 hours). This longer half-life provides a longer duration of action, lowering the risk of a second seizure or need for repeat dosing.

Accordingly, in some embodiments, administering a composition comprising an effective amount of diazepam, an alkyl maltoside, and a carrier system comprising one or more natural or synthetic tocopherols or tocotrienols and one or more alcohols as described herein to a nasal mucosal membrane of a subject during or before a seizure is effective to reduce clinical seizure activity in the subject for a period of at least 8 hours after said administering. In some embodiments, the clinical seizure activity is reduced for a period of at least 12 hours after said administering, a period of at least 18 hours after said administering, a period of at least 24 hours after said administering, a period of at least 36 hours after said administering, a period of at least 48 hours after said administering, or a period of >48 hours after said administering. The longer half-life and longer duration of action is particularly useful for the treatment of a subject experiencing a seizure cluster or acute repetitive seizures.

In some embodiments, administering a composition comprising an effective amount of diazepam as described herein to a nasal mucosal membrane of a subject during or before a seizure is effective to prevent a second seizure in the subject for a period of at least 8 hours after said administering. In some embodiments, the second seizure in the subject is prevented for a period of at least 12 hours after said administering, a period of at least 18 hours after said administering, a period of at least 24 hours after said administering, a period of at least 36 hours after said administering, a period of at least 48 hours after said administering, or a period of >48 hours after said administering. The prevention of a second seizure is particularly beneficial for a subject experiencing a seizure cluster or acute repetitive seizures.

In some embodiments, administering a composition comprising an effective amount of diazepam as described herein to a nasal mucosal membrane of a subject during or before a seizure is effective to reduce the number of Detections per hour in a subject by about 25% as compared to the subject's baseline number of Detections per hour. As described in more detail in the Examples herein, “Detections” are electrographic spikes of a pre-determined character that can be detected and recorded. Suitable devices for detecting and recording baseline Detection levels and Detection levels after treatment include, without limitation, an electroencephalogram (EEG), a responsive neurostimulation (RNS) device, a portable EEG headband (e.g., MUSE from InteraXon, Inc. Toronto, Canada), a wrist-worn actigraphy device (e.g., Actiwatch 2 from Philips Respironics, Murrysville, Pa.), forehead worn sleep monitor (e.g., Sleep Profiler from Advance Brain Monitoring, Carlsbad Calif.), and non-contact sleep sensor (e.g., Beddit 3 Sleep monitoring system from Apple, Cupertino Calif.). Thus, in some embodiments, the subject has a device capable of detecting and recording the Detections, e.g., the subject has an RNS device implant, and the device is capable of identifying a baseline Detection rate and a reduction in the Detection rate in the subject following administration of a composition as described herein. A suitable baseline Detection per hour rate for a subject can be established as described herein, e.g., the mean hourly Detection rate over a defined pre-administration observation period (e.g., a 3-, 4-, 5-, 6-, 7-day or longer pre-administration observation period).

In some embodiments, administering the composition as described herein reduces Detections per hour in a subject by about 30%, by about 35%, by about 40%, by about 45%, by about 50%, by >50% as compared to the subject's baseline average Detections per hour. In some embodiments, the reduction in Detections per hour is maintained over an 8-hour period, over a 12-hour period, over an 18-hour period, over a 24-hour period, over a 36-hour period, over a 48-hour period, or for a period of time extending beyond 48-hours.

In some embodiments, administering a composition comprising an effective amount of diazepam as described herein to a nasal mucosal membrane of a subject during or before a seizure is effective to reduce the number of Long Episodes experienced by the subject over a 24-hour period as compared to the subject's baseline number of Long Episodes over a 24-hour period. As described in the Examples herein, “Long Episodes” are more complex Detections that correlate with the occurrence of a clinical seizure. In accordance with this embodiment, the number of Long Episodes experience by a subject over a 24-hour period may be reduced by 1, 2, 3, 4, 5, 6, 7, 8, 9, 10 or >10 over a 24-hour period. In some embodiments, the reduction in Long Episodes is maintained over a 36-hour period, over a 48-hour period, or over a >48-hour period. Similar to Detections, the Long Episodes can be detected, recorded, monitored, etc. using suitable devices. Thus, in some embodiments, the subject has a device capable of detecting and recording Long Episodes (e.g., the subject has an RNS device implant), and, in particular, the reduction in the Long Episodes after administration of the composition described herein.

In some embodiments, administering a composition comprising an effective amount of diazepam as described herein to a nasal mucosal membrane of a subject during or before a seizure is effective to preclude the need for a second administration of the composition within 24-hours of experiencing the first seizure. In some embodiments, the dose is effective to preclude the need for a second administration of the composition within 48-hours or more of experiencing the first seizure.

Equally surprising is that a therapeutic benefit of the disclosed intranasal compositions is realized in a subject prior to PK-based predictions. For example, a therapeutic benefit may be realized in a subject within 10 minutes of intranasal administration of a benzodiazepine via an intranasal composition as disclosed herein, such as within 5 minutes, within 1 minute, within about 30 seconds to 4 minutes, within about 30 seconds to about 3 minutes, within about 30 seconds to about 2 minutes, or within about 30 seconds to about 1 minute after intranasal administration. Examples of therapeutic benefits that may be realized in a subject include, but are not limited to, experiencing no seizure, a less-severe seizure, or a shorter seizure as compared to a subject who is administered a benzodiazepine orally or intravenously.

Therefore, in another aspect, the present disclosure provides a method of preventing (i.e., inhibiting the onset) or treating a seizure in a subject in need thereof and effecting a reduction in severity of the seizure within about 10 minutes after administration of an intranasal composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure. For example, an intranasal composition may comprise an effective amount of a benzodiazepine (such as about 5 mg to about 20 mg of diazepam), about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 51.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of recurrent seizures in the subject.

Quantitatively, a therapeutic effect of a benzodiazepine administered via an intranasal composition, as disclosed herein, on seizure activity can be detected by monitoring beta rhythm or beta frequency by EEG. Changes in beta frequency, after intranasal administration of 20 mg of diazepam via an intranasal composition, as disclosed herein, have been observed within about 6 minutes after intranasal administration. In comparison, beta frequency after oral administration of 10 mg diazepam does not change until about 1.5 to 2 hours (see, e.g., Friedman, et al., as disclosed above). The divergence of PD and PK profiles, more particularly, the observation that the PK profile of diazepam administered according to the methods disclosed herein mimics the Tₘₐₓ and Cₘₐₓ of oral dosage forms but has the bioavailability and rapid onset of therapeutic benefit of a benzodiazepine administered intravenously or rectally, is surprising and unexpected. Despite similar systemic drug concentrations, therapeutic benefit evidenced in both cessation of seizure as well as a change in measurable beta frequency, is achieved more rapidly using the intranasal compositions disclosed herein than in oral dosage forms. Additionally, therapeutic benefits are realized in a timeframe similar to IV administration while avoiding the high and dangerous systemic circulation concentrations.

Therefore, in another aspect, the present disclosure provides a method of preventing (i.e., inhibiting the onset) or treating a seizure in a subject comprising modulating the beta frequency of the subject by administering a composition comprising an effective amount of a benzodiazepine (e.g., diazepam), about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure. For example, an intranasal composition may comprise an effective amount of a benzodiazepine (such as about 5 mg to about 20 mg of diazepam), about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam benzodiazepine, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of recurrent seizures in the subject. Changes in beta frequency in a subject following administration of a benzodiazepine (e.g., diazepam) via the intranasal composition to the subject may occur within about 30 minutes, within about 25 minutes, within about 20 minutes, within about 15 minutes, within about 10 minutes, or within about 5 minutes after administration of the benzodiazepine.

Qualitatively (or semi-quantitatively), a therapeutic effect of a benzodiazepine administered via an intranasal composition, as disclosed herein, on seizure activity may be measured using a Quality of Life in Epilepsy Scale (QOLIE) assessment by a subject. QOLIE scores are based on individual sub scores in categories of each of Seizure Worry, Overall QoL, Emotional Well-Being, Energy/Fatigue, Cognitive Functioning, Medication Effects, Social Functioning) were analyzed in frequent and infrequent users of diazepam nasal spray; in particular, Seizure Worry and Social Functioning. Advantageously, QOLIE scores of subjects being treated for seizure clusters with benzodiazepine via an intranasal composition, as disclosed here, are maintained or improved with ongoing treatment. Therefore, in another aspect, the present disclosure provides a method of treating cluster seizures, which may optionally be refractory, in a subject comprising by administering a composition comprising an effective amount of a benzodiazepine (e.g., diazepam), about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure. For example, an intranasal composition may comprise an effective amount of a benzodiazepine (such as about 5 mg to about 20 mg of diazepam), about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam benzodiazepine, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of cluster seizures in the subject and to maintain or improve the subject's quality of life, as measured by a QOLIE assessment.

Advantageously, a benzodiazepine, such as diazepam, may be administered to a subject via an intranasal composition, as disclosed herein, in combination with a second benzodiazepine, such as clobazam without adversely affecting the efficacy of the benzodiazepine in the intranasal composition. As such, in another aspect, the present disclosure provides a method of preventing (i.e., inhibiting the onset) or treating a seizure in a subject in need thereof comprising administering to the subject a composition comprising an effective amount of a first benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure, wherein the subject is under treatment with a second benzodiazepine. In any embodiment, for example, the first benzodiazepine may be diazepam and the second benzodiazepine may be clobazam. For example, an intranasal composition may comprise an effective amount of a first benzodiazepine (such as about 5 mg to about 20 mg of diazepam), about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 The second benzodiazepine may be administered via any suitable route, such as orally. In accordance with the methods disclosed herein, the administering is carried out to reduce one or more of the frequency, length, and severity of recurrent seizures in the subject, and may have an added benefit of reduced euphoria thereafter.

Approximately one third of pediatric patients with epilepsy develop refractory epilepsy that can be associated with medically resistant seizures (with or without clustering) and impaired development. Advantageously, benzodiazepine administered via an intranasal composition as disclosed herein may be effective to treat medically resistant seizures in subject with epilepsy that are aged 6 or older. As such, in another aspect, the present disclosure provides a method of treating refractory epilepsy in a subject in need thereof comprising administering to the subject a composition comprising an effective amount of a first benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure. For example, an intranasal composition may comprise an effective amount of a first benzodiazepine (such as about 5 mg to about 20 mg of diazepam), about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of recurrent seizures in the subject, and may have an added benefit of reduced euphoria thereafter.

The systemic presence of benzodiazepines is often accompanied by adverse effects such as, but not limited to, somnolence, euphoria, headache/migraine, suicidal ideation or behavior, depression, vasodilation, diarrhea, ataxia, dizziness, incoordination, rash, asthma, confusion, slurred speech, muscle weakness, memory problems, dry mouth, constipation, and blurred vision. As such, compliance by a subject with treatment regimens is often poor, as a subject wishes to avoid adverse effects. Additionally, a subject may delay systemic administration in an attempt to avoid the adverse effects.

Surprisingly, subjects treated with benzodiazepine via an intranasal composition, as disclosed herein, report a lower incidence and/or a reduced severity of many of these aforementioned adverse effects, such as somnolence, euphoria, headache, suicidal ideation and behavior, depression, incoordination, rash, asthma, and vasodilation. This is particularly surprising in view of exhibiting a benzodiazepine bioavailability in a subject that rivals the bioavailability observed after direct systemic administration. Therefore, a subject undergoing treatment using the intranasal compositions, as disclosed herein, may be less likely to discontinue use or delay use of the composition to treat a seizure as compared to a subject using a benzodiazepine composition administered orally, intravenously, or rectally.

As such, in another aspect, the present disclosure provides a method of preventing (i.e., inhibiting the onset) or treating a seizure in a subject in need thereof comprising administering to the subject a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure, wherein the subject experiences a reduced incidence or severity of euphoria after the administering. For example, the incidence or severity of euphoria may be reduced in the subject by about 5%, about 10%, about 15%, about 20% about 25%, about 30%, about 35%, about 40%, about 45%, about 50%, or greater than 50% relative to euphoria experienced after administration of a therapeutically effective amount of diazepam via rectal, intravenous, or oral administration. For example, an intranasal composition may comprise an effective amount of a benzodiazepine (such as about 5 mg to about 20 mg of diazepam), about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of recurrent seizures in the subject, and may have an added benefit of reduced euphoria thereafter.

In another aspect, the present disclosure provides a method of preventing (i.e., inhibiting the onset) or treating a seizure in a subject in need thereof comprising administering to the subject a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure, wherein the subject experiences a reduced incidence or severity of headache after the administering. For example, the incidence or severity of headaches may be reduced in the subject by about 5%, about 10%, about 15%, about 20% about 25%, about 30%, about 35%, about 40%, about 45%, about 50%, or greater than 50% relative to headaches experienced after administration of a therapeutically effective amount of diazepam via rectal, intravenous, or oral administration. For example, an intranasal composition, as disclosed herein, may comprise an effective amount of a benzodiazepine (such as about 5 mg to about 20 mg of diazepam), about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of recurrent seizures in the subject, and may have an added benefit of reduced headache severity or length thereafter.

In another aspect, the present disclosure provides a method of preventing (i.e., inhibiting the onset) or treating a seizure in a subject in need thereof comprising administering to the subject a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure, wherein the subject experiences a reduced incidence or severity of suicidal thoughts, behaviors, or tendencies after the administering. For example, the incidence or severity of suicidal thought, behavior, or tendency may be reduced in the subject by about 5%, about 10%, about 15%, about 20% about 25%, about 30%, about 35%, about 40%, about 45%, about 50%, or greater than 50% relative to suicidal thought, behavior, or tendency experienced after administration of a therapeutically effective amount of diazepam via rectal, intravenous, or oral administration. For example, an intranasal composition, as disclosed herein, may comprise an effective amount of a benzodiazepine (such as about 5 mg to about 20 mg of diazepam), about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of recurrent seizures in the subject, and may have an added benefit of reduced suicidal thought, behavior, or tendency thereafter.

In another aspect, the present disclosure provides a method of preventing (i.e., inhibiting the onset) or treating a seizure in a subject in need thereof comprising administering to the subject a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure, wherein the subject experiences a reduced incidence or severity of depression after the administering. For example, the incidence or severity of depression may be reduced in the subject by about 5%, about 10%, about 15%, about 20% about 25%, about 30%, about 35%, about 40%, about 45%, about 50%, or greater than 50% relative to depression experienced after administration of a therapeutically effective amount of diazepam via rectal, intravenous, or oral administration. For example, an intranasal composition, as disclosed herein, may comprise an effective amount of a benzodiazepine (such as about 5 mg to about 20 mg of diazepam), about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of recurrent seizures in the subject, and may have an added benefit of reduced depression thereafter.

In another aspect, the present disclosure provides a method of preventing (i.e., inhibiting the onset) or treating a seizure in a subject in need thereof comprising administering to the subject a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure, wherein the subject experiences a reduced incidence or severity of somnolence after the administering. For example, the incidence or severity of somnolence may be reduced in the subject by about 5%, about 10%, about 15%, about 20% about 25%, about 30%, about 35%, about 40%, about 45%, about 50%, or greater than 50% relative to somnolence experienced after administration of a therapeutically effective amount of diazepam via rectal, intravenous, or oral administration. For example, an intranasal composition, as disclosed herein, may comprise an effective amount of a benzodiazepine (such as about 5 mg to about 20 mg of diazepam), about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of recurrent seizures in the subject, and may have an added benefit of reduced somnolence thereafter.

Having reduced incidence of adverse effects, compliance of a treatment regimen using the compositions disclosed herein by a subject may be more complete when compared to treatment regimen using an alternate benzodiazepine composition. Therefore, in another aspect, the present disclosure provides a method of improving patient compliance with a prescribed treatment regimen for preventing (i.e., inhibiting the onset) or treating a seizure, wherein the treatment method comprises intranasally administering a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure. For example, an intranasal composition, as disclosed herein, may comprise an effective amount of a benzodiazepine (such as about 5 mg to about 20 mg of diazepam), about 0.01% w/v to about 1% w/v of an alkyl maltoside, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with this aspect of the disclosure, the administering is carried out to reduce one or more of the frequency, length, and severity of the recurrent seizure in the subject.

Compliance of a treatment regimen using the single dose compositions disclosed herein by a subject may be more complete when compared to treatment regimen using an alternate, multi-dose benzodiazepine composition. Therefore, in another aspect, the present disclosure provides a method of improving patient compliance with a prescribed treatment regimen for preventing (i.e., inhibiting the onset) or treating a seizure, wherein the treatment method comprises intranasally administering a single dose composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure. In some embodiments, the single dose composition comprises about 20% w/v of diazepam, about 35% to about 45% w/v vitamin E, about 0.25% DDM, and about 35% to about 45% benzyl alcohol in the desired volume (e.g., 100 μL). In some embodiments, the single dose composition comprises about 20% w/v of diazepam, about 35% to about 45% w/v vitamin E, about 0.50% DDM, and about 35% to about 45% benzyl alcohol in the desired volume (e.g., 100 μL). In some embodiments, the single dose composition comprises about 20% w/v of diazepam, about 35% to about 45% w/v vitamin E, about 0.75% DDM, and about 35% to about 45% benzyl alcohol in the desired volume (e.g., 100 μL). In accordance with this aspect of the disclosure, the administering is carried out to reduce one or more of the frequency, length, and severity of the recurrent seizure in the subject.

Compliance with a prescribed treatment regimen is important in effectively treating recurrent seizures in a subject. Recurrent seizures, such as those that occur in epilepsy syndromes, that are left under-treated, mistreated, or not treated may, after repetitive seizure episodes, result in neurological damage. For example, evidence of progressive damage in temporal lobe epilepsy associated with hippocampal sclerosis (TLE-HS) has been identified through neuroimaging and electroencephalography (see, e.g., Coan, A. C., and Cendes, F.; Epilepsy & Behavior; March 2013, 26(3): pages 313-321, which is incorporated herein in its entirety by reference). Pathologically, excessive neuronal excitability characterizing a seizure results in massive depolarization of neurons, excessive glutamate release, and increased intracellular calcium which causes a cascade of changes that ultimately result in cell death (see, e.g., Holmes, G. L.; Neurology; Nov. 12, 2002, 59 (9 Suppl. 5), which is incorporated in its entirety herein by reference). The deleterious effects of recurrent seizures may be evidenced, for example, by an increase in cognitive dysfunction, an increase in severity, length, or frequency of seizures. However, administration of a benzodiazepine via the intranasal compositions as disclosed herein, advantageously, result in slowed or halted cognitive decline and/or decreased severity, length, or frequency of recurrent seizures when compared to the severity, length, and frequency of recurrent seizures in a subject left un-treated or treated via a different administration route.

Thus, in another aspect, the present disclosure provides a method of treating recurrent seizures in a subject comprising administering to a subject in need thereof a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In some embodiments, a method of treating recurrent seizures in a subject comprises administering a single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.25% (w/v). In some embodiments, a method of treating recurrent seizures in a subject comprises administering a single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.50% (w/v). In some embodiments, a method of treating recurrent seizures in a subject comprises administering a single dose intranasal composition comprising diazepam or a pharmaceutically acceptable salt thereof in an amount of about 20% (w/v); vitamin E in an amount from about 35% to about 45% (w/v); benzyl alcohol in an amount from about 35% to about 45% (w/v); and n-dodecyl beta D-maltoside (DDM) in an amount of about 0.75% (w/v). In accordance with the methods disclosed herein, the administering is carried out to reduce one or more of the frequency, length, and severity of recurrent seizures in the subject.

As seasonal allergies are associated with nasal inflammation, it is important to establish that the safety of intranasal seizure rescue medications is not impacted by seasonal allergies. Advantageously, seasonal allergies or rhinitis in a patient does not increase the number of treatment-emergent adverse effects associated with the administration of benzodiazepine via an intranasal composition, as disclosed herein. Thus, in another aspect, the present disclosure provides a method of treating recurrent seizures in a subject suffering from seasonal allergies or rhinitis, the method comprising administering to a subject in need thereof a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of recurrent seizures in the subject suffering from seasonal allergies or rhinitis.

Administering a benzodiazepine via an intranasal composition, as disclosed herein, to a subject may, in any embodiment, reduce neuronal loss caused by the recurrent seizures in a subject that would otherwise occur in the subject absent treatment or being treated with another benzodiazepine dosage form. For example, a 1% reduction, 2% reduction, 3% reduction, 4% reduction, 5% reduction, 6% reduction, 7% reduction, 8% reduction, 9% reduction, 10% reduction, 11% reduction, 12% reduction, 13% reduction, 14% reduction, 15% reduction, 16% reduction, 17% reduction, 18% reduction, 19% reduction, or a reduction greater than 20% in neuronal loss is observed in the subject following administration of a benzodiazepine via an intranasal composition, as disclosed herein, as compared to the neuronal loss that would otherwise occur absent treatment or being treated with another benzodiazepine dosage form.

Administering a benzodiazepine via an intranasal composition, as disclosed herein, for the treatment of recurrent seizures may, in any embodiment, reduce or prevent cognitive dysfunction caused by the recurrent seizures in a subject that would otherwise occur in the subject absent treatment or being treated with another benzodiazepine dosage form. In any embodiment, a 1% reduction, 2% reduction, 3% reduction, 4% reduction, 5% reduction, 6% reduction, 7% reduction, 8% reduction, 9% reduction, 10% reduction, 11% reduction, 12% reduction, 13% reduction, 14% reduction, 15% reduction, 16% reduction, 17% reduction, 18% reduction, 19% reduction, or a reduction greater than 20% in cognitive dysfunction is observed in the subject following administration of a benzodiazepine via an intranasal composition, as disclosed herein, as compared to cognitive dysfunction that would otherwise occur in the subject absent treatment or being treated with another benzodiazepine dosage form.

Dosage forms of benzodiazepines currently available have associated with them a variety of contraindications due in part to the various adverse effects that have been observed with the administration of the dosage forms. For example, the administration of diazepam is contraindicated in subjects with glaucoma (especially acute narrow-angle glaucoma and untreated open-angle glaucoma) due to the possibility of increased intraocular pressure. With the reduced incidence of adverse effects, the intranasal compositions disclosed herein can be safely and effectively administered to subjects with glaucoma to treat a co-morbid seizure or seizure disorder or syndrome. Accordingly, in another aspect, the present disclosure provides a method of treating or preventing (i.e., inhibiting the onset) a seizure in a subject having glaucoma, the method comprising administering to the subject a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In some embodiments, the subject has narrow angle glaucoma.

Co-administration of benzodiazepines with opioids is also contraindicated as both drugs sedate users and suppress breathing, leading to a higher risk for overdose and death due to respiratory distress. With the reduced incidence of adverse effects, the intranasal compositions disclosed herein can, in some instances, be safely and effectively administered to subjects undergoing treatment involving opioid administration. Accordingly, in another aspect, the present disclosure provides a method of treating or preventing (i.e., inhibiting the onset) a seizure in a subject taking prescription opioids, the method comprising administering a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL).

Chronic administration of benzodiazepines to treat seizures (e.g., administration of benzodiazepines more than five times per month), is also contraindicated in oral, intravenous, and rectal formulations of benzodiazepines due to the propensity to develop tolerance and/or dependence with the repeated systemic exposure to the benzodiazepine. Advantageously, the intranasal compositions disclosed herein do not induce tolerance or dependence within a subject, even when administered chronically to treat recurrent seizures, such as within 4 hours, 5 hours, 6 hours, 12 hours, 15 hours after a first dose and may be administered, on a monthly basis, up to at least 6 times a month, more than 10 times a month, or more than 15 times a month. Therefore, the intranasal compositions disclosed herein may be used to prevent or acutely treat a seizure without regard to prior administrations and the timing thereof.

Therefore, in another aspect, the present disclosure provides a method of preventing (i.e., inhibiting the onset) or treating a seizure within a series of recurrent seizures, the method comprising administering a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before each of the recurrent seizures, wherein 6, 7, 8, 9, 10, 11, 12, 13, 14, 15, 16, 17, 18, 19, or more seizures occur per month. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of recurrent seizures in the subject.

Further, and advantageously, the intranasal compositions disclosed herein do not induce any treatment-emergent adverse effects when administered as a second dose to treat a seizure event that was not adequately treated with a first dose. For example, a second dose, which may be the same strength or different than the first dose, may be administered 0.5 to 12 hours, such as 0.5 hours, 1 hour, 2 hours, 3 hours, 4 hours, or more than 4 hours after the first dose, if needed, to treat ongoing seizure clusters.

Therefore, in another aspect, the present disclosure provides a method of preventing (i.e., inhibiting the onset) or treating cluster seizures, the method comprising administering a first dose of a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure cluster, wherein the seizure cluster is not adequately treated with the first dose and wherein a second dose of the composition is administered to the nasal mucosal membrane of the subject after a period of at least 0.5 hours after the first dose. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of cluster seizures in the subject.

Seizure clusters, or acute repetitive seizures, are emergencies associated with increased risk of prolonged seizures and status epilepticus, requiring prompt treatment to lower the risk of associated morbidities. The time frame between seizure clusters is known as the interseizure cluster interval (ISCI). Advantageously and surprisingly, continued administration of a benzodiazepine via the intranasal compositions, as disclosed herein, to treat seizure clusters results in an increase in the duration of the ISCI in subjects. An increased ISCI duration ultimately leads to fewer seizure clusters, and thus fewer seizures, for a subject.

Therefore, in another aspect, the present disclosure provides a method of increasing the duration of an ISCI in a subject in need thereof, the method comprising administering a composition comprising an effective amount of a benzodiazepine to subject. In some embodiments, a method of treating seizure clusters in a subject in need thereof is provided, the method comprising administering to the subject a composition comprising an effective amount of a benzodiazepine, wherein administration of the composition increases the length of an interseizure cluster interval (ISCI) in the subject. In some embodiments, a method of increasing the time to a seizure cluster in a subject suffering from recurrent seizure clusters is provided, the method comprising administering to the subject a composition comprising an effective amount of a benzodiazepine, wherein administration of the composition increases the length of an ISCI in the subject. In some embodiments, the composition is an oral, intravenous, rectal, or intranasal formulation. In some embodiments, the composition is an intranasal formulation administered to a nasal mucosal membrane of the subject.

In some embodiments, the composition comprises about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure cluster, wherein the seizure cluster is not adequately treated with the first dose and wherein a second dose of the composition is administered to the nasal mucosal membrane of the subject after a period of at least 0.5 hours after the first dose. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL).

Increasing the duration of an ISCI in a subject with seizure clusters requires administering composition comprising an effective amount of a benzodiazepine before, during, or after a seizure within a seizure cluster or acute repetitive seizures. Therefore, in another aspect, the present disclosure provides a method of increasing the duration of an ISCI in a subject in need thereof or increasing the time to a seizure cluster in a subject suffering recurrent seizure clusters, the method comprising administering at least two or more doses of a composition comprising an effective amount of a benzodiazepine to a nasal mucosal membrane of the subject. In some embodiments, the composition comprises about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure cluster, wherein the seizure cluster is not adequately treated with the first dose and wherein a second dose of the composition is administered to the nasal mucosal membrane of the subject after a period of at least 0.5 hours after the first dose. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL).

In some embodiments, the ISCI duration in a subject will continue to increase as treatment progresses. For example, subjects suffering from seizure clusters who use an intranasal benzodiazepine composition, saw averages increased in ISCI duration when monitored over at least a three-month period. Therefore, in another aspect, the present disclosure provides a method of increasing the duration of an ISCI in a subject in need thereof, or increasing the time to a seizure cluster in a subject suffering recurrent seizure clusters, over at least a three-month period, the method comprising administering at least two or more doses of a composition comprising an effective amount of a benzodiazepine to a nasal mucosal membrane of the subject. In some embodiments, the composition comprises about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure cluster, wherein the seizure cluster is not adequately treated with the first dose and wherein a second dose of the composition is administered to the nasal mucosal membrane of the subject after a period of at least 0.5 hours after the first dose. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL).

In some embodiments, the increase in ISCI duration can be seen in an outright decrease in the frequency of seizure clusters over a period of time, for example, a year. Therefore, in another aspect, the present disclosure provides a method of decreasing the frequency of seizure clusters in a subject suffering recurrent seizure clusters, the method comprising administering at least two or more doses of a composition comprising an effective amount of a benzodiazepine to a nasal mucosal membrane of the subject. In some embodiments, the composition comprises about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure cluster, wherein the seizure cluster is not adequately treated with the first dose and wherein a second dose of the composition is administered to the nasal mucosal membrane of the subject after a period of at least 0.5 hours after the first dose. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL).

An effective amount of a benzodiazepine may be administered to a subject via an intranasal composition, as disclosed herein, multiple times, if necessary, to effectively treat or prevent a seizure. Even if a subject requires multiple treatments to effectively treat or prevent a seizure within a seizure cluster, the subject can still see an increase in ISCI duration with continued use of the intranasal benzodiazepine composition. As such, in another aspect, the present disclosure provides a method of increasing the duration of an ISCI in a subject in need thereof, or increasing the time to a seizure cluster in a subject suffering recurrent seizure clusters, the method comprising administering a first dose of a composition comprising an effective amount of a benzodiazepine to a nasal mucosal membrane of the subject during or before a seizure, wherein when adequate cessation or prevention of the seizure is not achieved within 4 hours, 2 hours, 1 hour, 30 minutes, 15 minutes, or 10 minutes after the administering of the first dose, one or more subsequent doses of the composition are administered to the subject. In some embodiments, the composition comprises about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure cluster, wherein the seizure cluster is not adequately treated with the first dose and wherein a second dose of the composition is administered to the nasal mucosal membrane of the subject after a period of at least 0.5 hours after the first dose. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL).

In any embodiment, an intranasal composition can comprise an effective amount of diazepam. Effective amounts of diazepam that may be administered via the intranasal compositions, as disclosed herein, include about 5 mg to about 20 mg of diazepam, such as about 5 mg to about 15 mg of diazepam, about 5 mg to about 10 mg of diazepam, about 10 mg to about 20 mg of diazepam, or about 15 mg to about 20 mg. Effective dosing may, in any embodiment, be determined based on the body weight of the subject to which the diazepam will be administered. For example, for any given subject having a body weight (in kg), a dose of about 0.25 mg/kg to about 0.60 mg/kg may be administered. For example, suitable doses include about 0.27 mg/kg, about 0.35 mg/kg, about 0.40 mg/kg, about 0.50 mg/kg, or about 0.55 mg/kg of body weight may be administered to a subject. Diazepam may be included in a composition comprising an about 0.01% w/v to about 1% w/v of an alkyl maltoside and a carrier system comprising about 30% w/v to about 90% w/v of a natural or synthetic tocopherol, a natural or synthetic tocotrienol, or a combination thereof, and about 10% w/v to about 70% w/v, the intranasal composition having a volume of about 10 μL to about 200 μL, about 50 μL to about 150 μL, about 75 μL to about 125 μL, about 75 μL, about 100 μL, or about 125 μL. For example, 5 mg, 7.5 mg, 10 mg, 15 mg, or 20 mg diazepam may be administered in a volume of about 75 μL, 100 μL, or 125 μL. A dose may be administered to a single nostril or split up between nostrils. For example, a dose of 5 mg diazepam in 100 μL, 7.5 mg diazepam in 100 μL, 10 mg diazepam in 100 μL, or 20 mg diazepam in 100 μL may be administered in a single nostril. A dose of 10 mg may alternatively be administered as a dose of 5 mg diazepam in 100 μL in each nostril. Similarly, a 15 mg dose may be administered, for example, as 7.5 mg diazepam in 100 μL to each nostril. A 20 mg dose may be administered, for example, 10 mg diazepam in 100 μL to each nostril.

In one embodiment, the intranasal composition administered in accordance with any of the methods disclosed herein comprises 5% w/v benzodiazepine, 56.5% w/v vitamin E, 0.25% w/v DDM, 10.5% w/v benzyl alcohol, and an amount of ethanol sufficient to reach the desired volume (e.g., 100 μL).

In one embodiment, the intranasal composition administered in accordance with any of the methods disclosed herein comprises 7.5% w/v benzodiazepine, 56.5% w/v vitamin E, 0.25% w/v DDM, 10.5% w/v benzyl alcohol, and an amount of ethanol sufficient to reach the desired volume (e.g., 100 μL). In one embodiment, the intranasal composition administered in accordance with any of the methods disclosed herein comprises 10% w/v benzodiazepine, 56.5% w/v vitamin E, 0.25% w/v DDM, 10.5% w/v benzyl alcohol, and an amount of ethanol sufficient to reach the desired volume (e.g., 100 μL). In one embodiment, the intranasal composition administered in accordance with any of the methods disclosed herein comprises 20% w/v benzodiazepine, about 35% to about 45% w/v vitamin E, about 0.25% DDM, and about 35 to about 45% benzyl alcohol in the desired volume (e.g., 100 μL). In one embodiment, the intranasal composition administered in accordance with any of the methods disclosed herein comprises 20% w/v benzodiazepine, about 35% to about 45% w/v vitamin E, about 0.5% DDM, and about 35 to about 45% benzyl alcohol in the desired volume (e.g., 100 μL). In one embodiment, the intranasal composition administered in accordance with any of the methods disclosed herein comprises 20% w/v benzodiazepine, about 35% to about 45% w/v vitamin E, about 0.75% DDM, and about 35 to about 45% benzyl alcohol in the desired volume (e.g., 100 μL).

In addition to being suitability for subjects ages 6 and above, the compositions disclosed herein are suitable for administration in younger patient populations, such as 2-5 year-olds. Therefore, in another aspect, the present disclosure provides a method of preventing (i.e., inhibiting the onset) or treating a seizure in a subject that is 2 years old, 3 years old, 4 years old, 5 years old, or older wherein the treatment method comprises administering a composition comprising an effective amount of a benzodiazepine, about 0.01% w/v to about 1% w/v of an alkyl maltoside (e.g., DDM or TDM), and a carrier system comprising about 30% w/v to about 90% w/v of a natural or synthetic tocopherol, a natural or synthetic tocotrienol, or a combination thereof and about 10% w/v to about 70% w/v of one or more alcohols to a nasal mucosal membrane of the subject during or before a seizure. For example, an intranasal composition, as disclosed herein, may comprise about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the composition comprises about 5% to about 20% w/v of diazepam, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL). In accordance with the methods disclosed herein, the administering is carried out to reduce one or more of the frequency, length, and severity of recurrent seizures in the subject.

Compared to other routes of administration, such as oral, intravenous, or rectal, the compositions disclosed herein may be administered in a much simpler manner through a nasal spray. Therefore, the subject themselves, particularly if in a prodromal or pre-ictal phase of a seizure, can effectively self-administer any intranasal composition, as disclosed herein, to their nasal mucosal membrane. Additionally, a caregiver need not be a trained medical professional to effectively administer the composition to the subject, as required, for example, in intravenous administration. Therefore, advantageously, the compositions disclosed herein provide an easily administrable dosage form that may be administered before or during a seizure for rapid and quick prevention or treatment of the seizure that has the capacity, in some instances, to provide long-term benefits.

### Therapeutic Systems

Another aspect of the disclosure relates to a therapeutic system for the treatment of a seizure condition, disorder, syndrome, or disease. This therapeutic system comprises a neurological monitoring device and an intranasal benzodiazepine composition as described herein. The neurological monitoring device may comprise a smart watch (e.g., Embrace by EMPATICA™, Inc.), an adhesive sensor applied externally to the skin (e.g., the BioStamp by MC10), an implantable detecting device, an electrode patch (e.g., SPEAC System by Brain Sentinel), or any other sensing device suitable for detecting changes in neurological or other physiological parameters indicative of an impending seizure. Alternatively, and/or in addition to the monitoring device and algorithm for predicting the onset of a seizure effect can also be employed or included in this therapeutic system (see e.g., Dauod and Bayoumi, “Efficient Epileptic Seizure Prediction Based on Deep Learning,” IEEE Trans. Biomed. Circuits Systems 13(5): 804 (2019), which is hereby incorporated by reference in its entirety).

In accordance with this aspect of the disclosure, the intranasal benzodiazepine composition of the system comprises about 5 mg to about 20 mg of diazepam, about 55% w/v to about 70% w/v of a natural or synthetic tocopherol or tocotrienol, and a mixture of about 5% w/v to about 15% w/v benzyl alcohol and about 10% w/v to about 25% w/v ethanol. In some embodiments, the intranasal benzodiazepine composition comprises about 5% w/v, about 10% w/v, or about 20% w/v benzodiazepine, about 56.5% w/v vitamin E, about 0.25% DDM, about 10.5% benzyl alcohol, and an amount of ethanol sufficient to reach the desired volume (e.g., 100 μL). In some embodiments, the composition comprises about 5% to about 20% w/v of diazepam, about 35% to about 45% w/v vitamin E, about 0.25% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.50% DDM, and about 35% to about 45% benzyl alcohol in the desired volume (e.g., 100 μL). In some embodiments, the composition comprises about 5% to about 20% w/v of diazepam, about 35% to about 45% w/v vitamin E, about 0.75% DDM, and about 35% to about 45% benzyl alcohol in the desired volume (e.g., 100 μL).

The therapeutic system herein comprises a suitable therapeutic regimen for treating a seizure condition, disorder, syndrome, or disease. Pairing the monitoring device with the intranasal benzodiazepine composition described herein allows for a subject suffering a seizure condition, disorder, syndrome, or disease to recognize the onset or impending onset of seizure and administer the intranasal benzodiazepine composition prior to or at the onset of the seizure to inhibit the onset of the seizure or reduce the frequency, length, and/or severity of the seizure.

The therapeutic system described herein is also suitable to employ in a method of preventing injuries that result from seizures, e.g., falls, and preventing ongoing or further injury to the brain in recurrent seizure syndromes. In some embodiments, the therapeutic system described herein is employed as a replacement or adjuvant therapy to chronic anti-epileptic drugs to reduce or avoid associate long-term adverse effects of those drugs.

### Methods of Manufacture

In any embodiment, the composition for intranasal administration may be substantially free of benzodiazepine microparticles, nanoparticles or combinations thereof. In any embodiment, an intranasal composition, as disclosed herein, may be made by adding one or more benzodiazepine compounds to a mixture of the one or more natural or synthetic tocopherols or tocotrienols. The mixture is stirred until the one or more benzodiazepine drugs dissolve or are substantially dissolved. Next, the one or more alcohols or glycols, or any combinations thereof, are added to the composition. This composition may be stirred until a homogeneous composition is achieved.

The formulation process may be adjusted to take into consideration variations in the intranasal composition. For example, an intranasal composition comprising benzyl alcohol and ethanol may be prepared by first combining vitamin E, benzyl alcohol and ethanol, mixing until the ingredients are homogenous, adding an alkyl maltoside (e.g., DDM and/or TDM), and mixing until the alkyl maltoside is dissolved and the solution is homogenous. Benzodiazepine (e.g., diazepam) may be added after which the mixture may be brought to volume by adding ethanol (Q.S.) to achieve the final target weight of solution. For another example, an intranasal composition comprising benzyl alcohol may be prepared by first combining vitamin E and benzyl alcohol, mixing until the ingredients are homogenous, adding an alkyl maltoside (e.g., DDM and/or TDM), and mixing until the alkyl maltoside is dissolved and the solution is homogenous. Benzodiazepine (e.g., diazepam) may be added and dissolved by stirring after which the mixture may be brought to volume to achieve the final target weight of solution. Solutions manufactured according to this process may be prepared in different concentrations of diazepam, such as, but not limited to 50 mg/mL (5% w/v), 75 mg/mL (7.5% w/v), 100 mg/mL (10% w/v), 150 mg/mL (15% w/v) or 200 mg/mL (20% w/v). Being suitable for administration to the nasal mucosal membrane, an intranasal composition, as disclosed herein, may be formulated as a sprayable composition having a therapeutically effective amount of a benzodiazepine (e.g., 1 mg to about 20 mg of diazepam) in an intranasally-administrable volume, such as about 10 μL to about 200 about 50 μL to about 150 about 75 μL to about 125 about 75 about 100 or about 125 μL.

Therefore, in another aspect, the present disclosure provides a device adapted for intranasal delivery of any compositions disclosed herein. Such a device may be provided or supplied as a pre-primed device or may be primed by the subject before use. A device may be a metered device and/or a single-dose device or a multi-dose device, such as a bi-dose device. Such devices typically comprise a piston, a swirl chamber, an actuator and deliver a spray formed when a composition in the reservoir is forced out through the swirl chamber. Devices may be actuated by a subject by holding the device between a second and third finger with a thumb on the actuator. A device may additionally include a pressure point mechanism to ensure reproducibility of the actuation force and emitted plume/spray characteristics.

### EMBODIMENTS OF THE DISCLOSURE

Embodiments provided herein also include, but are not limited to, the following:

Embodiment 1. A method of treating a seizure in a subject in need thereof comprising intranasally administering a composition comprising an effective amount of diazepam, an alkyl maltoside, and a carrier system comprising one or more natural or synthetic tocopherols or tocotrienols and one or more alcohols, to a nasal mucosal membrane of the subject.

Embodiment 2. A method of inhibiting the onset of a seizure comprising administering a composition comprising an effective amount of diazepam, an alkyl maltoside, and a carrier system comprising one or more natural or synthetic tocopherols or tocotrienols and one or more alcohols to a nasal mucosal membrane of the subject during a prodromal or pre-ictal phase of the seizure.

Embodiment 3. A method of treating recurrent seizures in a subject in need thereof comprising administering a composition comprising an effective amount of diazepam, an alkyl maltoside, and a carrier system comprising one or more natural or synthetic tocopherols or tocotrienols and one or more alcohols to a nasal mucosal membrane of the subject before, during, or after a seizure.

Embodiment 4. The method of any one of embodiments 1-3, wherein the effective amount of diazepam is about 5 mg to about 20 mg diazepam in a volume of about 10 μL to 200 μL of the composition.

Embodiment 5. The method of any one of embodiments 1-4, wherein the effective amount of diazepam is about 5 mg to about 10 mg diazepam in a volume of about 10 μL to 200 μL of the composition.

Embodiment 6. The method of any one of embodiments 1-5, wherein the alkyl maltoside is selected from dodecyl maltoside, tetradecyl maltoside, or a combination thereof.

Embodiment 7. The method of any one of embodiments 1-6, wherein the composition comprises about 0.1% w/v to about 1% w/v of the alkyl maltoside.

Embodiment 8. The method of any one of embodiments 1-7, wherein the one or more alcohols comprises a mixture of ethanol and benzyl alcohol, or only benzyl alcohol.

Embodiment 9. The method of any one of embodiments 1-8, wherein the composition comprises a mixture of about 17% w/v to about 20% w/v ethanol and about 10% w/v to about 12% w/v benzyl alcohol; or only benzyl alcohol in an amount from about 35% to about 45% (w/v).

Embodiment 10. The method of any one of embodiments 1-9, wherein the severity of the seizure in the subject is reduced within 10 minutes after administration.

Embodiment 11. The method of any one of embodiments 1-10, wherein a change in beta frequency is measured in the subject within 2 minutes after administration.

Embodiment 12. The method of any one of embodiments 1-11, wherein the seizure is caused by epilepsy or an epileptic disorder.

Embodiment 13. The method of any one of embodiments 1-12, wherein the seizure is an absence seizure, a myoclonic seizure, a clonic seizure, a tonic seizure, a tonic-clonic seizure, an atonic seizure, a focal seizure, or any combination thereof.

Embodiment 14. The method of any one of embodiments 1-13, wherein the seizure is a seizure within a seizure cluster or acute repetitive seizures.

Embodiment 15. The method of any one of embodiments 1-14, wherein the administering is performed by the subject or a caregiver.

Embodiment 16. The method of any one of embodiments 1-15, wherein the administering is performed by the subject.

Embodiment 17. The method of any one of embodiments 1-16, wherein the administering is performed in a prodromal phase or pre-ictal phase of the seizure.

Embodiment 18. The method of any one of embodiments 1-17, wherein the administering is performed in an ictal phase of the seizure.

Embodiment 19. The method of any one of embodiments 1-18, wherein said administering achieves a Tₘₐₓ for diazepam of 1 hour or more.

Embodiment 20. The method of any one of embodiments 1-19, wherein after said administering, the subject experiences decreased incidence or severity of somnolence in comparison with somnolence experienced after administration of a therapeutically effective amount of diazepam via rectal, intravenous, or oral administration.

Embodiment 21. The method of any one of embodiments 1-20 further comprising: administering a second effective amount of the composition within 4 hours of said administering.

Embodiment 22. The method of any one of embodiments 1-21, wherein the administering the second effective amount of the composition is carried out within 2 hours of said administering.

Embodiment 23. The method of any one of embodiments 1-22, wherein the administering the second effective amount of the composition is carried out within 1 hour of said administering.

Embodiment 24. The method of embodiments 21, further comprising: administering a third effective amount of the composition within 4 hours of administering the second effective amount.

Embodiment 25. The method of any one of embodiments 1-24, wherein after said administering, the subject experiences decreased incidence or severity of euphoria in comparison with euphoria experienced after administration of a therapeutically effective amount of diazepam via rectal, intravenous, or oral administration.

Embodiment 26. The method of any one of embodiments 1-25, wherein after said administering, the subject experiences decreased incidence or severity of headache in comparison with headache experienced after administration of diazepam via rectal, intravenous, or oral administration.

Embodiment 27. The method of any one of embodiments 1-26, wherein after said administering, the subject experiences decreased suicidal ideation and behavior in comparison with suicidal ideation experienced after administration of a therapeutically effective amount of diazepam via rectal, intravenous, or oral administration.

Embodiment 28. The method of any one of embodiments 1-24, wherein after said administering, the subject experiences decreased depression in comparison with depression experienced after administration of a therapeutically effective amount of diazepam via rectal, intravenous, or oral administration.

Embodiment 29. The method of any one of embodiments 1-28, wherein the composition is provided in a pre-primed single use dosage device containing about 0.1 mL of the composition.

Embodiment 30. The method of any one of embodiments 1-29, wherein said administering comprises administering 0.1 mL of the composition to each nostril of the subject, or administering 0.1 mL of the composition to one nostril of the subject.

Embodiment 31. The method of any one of embodiments 1-30, wherein the subject suffers from narrow angle glaucoma.

Embodiment 32. The method of any one of embodiments 1-31, wherein the subject is undergoing treatment that comprises administration of an opioid.

Embodiment 33. The method of any one of embodiments 1-32, wherein the subject is 2-5 years old or older.

Embodiment 34. The method of any one of embodiments 1-33, wherein one or more of the frequency, length, and severity of the recurrent seizures is reduced.

Embodiment 35. The method of any one of embodiments 1-33, wherein the method reduces neuronal loss caused by the recurrent seizures.

Embodiment 36. The method of any one of embodiments 1-33, wherein the method slows or stops cognitive dysfunction caused by the recurrent seizures.

Embodiment 37. The method of any one of embodiments 1-33, wherein six or more seizures occur within a period of a month and the composition is administered during at least six of the seizures.

Embodiment 38. The method of any one of embodiments 1-37, wherein after said administering, the subject is less likely to discontinue such treatment with said composition in comparison to the likelihood of discontinuing treatment after administration of a therapeutically effective amount of diazepam via rectal, intravenous, or oral administration.

Embodiment 39. The method of any one of embodiments 1-38, wherein after said administering, the subject is less likely to develop tolerance to said composition in comparison to the likelihood of developing tolerance after administration of a therapeutically effective amount of diazepam via rectal, intravenous, or oral administration.

Embodiment 40. A method of increasing the time to a second seizure in a subject suffering from recurrent seizures, said method comprising: administering a composition comprising an effective amount of diazepam, an alkyl maltoside, and a carrier system comprising one or more natural or synthetic tocopherols or tocotrienols and one or more alcohols to a nasal mucosal membrane of the subject during or before a first seizure.

Embodiment 41: The method of any one of embodiments 1-40, wherein the subject has been diagnosed with or suspects that they suffer from seasonal allergies.

Embodiment 42. The method of any one of embodiments 1-41, wherein the subject has been diagnosed with refractory epilepsy.

Embodiment 43. The method of any one of embodiments 1-42, further comprising administering clobazam to the subject.

Embodiment 44. The method of any one of embodiments 1-43, wherein the administration causes mild to no nasal irritation.

Embodiment 45. The method of any one of embodiments 1-44, wherein administrating maintains or improve the subject's quality of life, as measured by a QOLIE assessment.

Embodiment 46. A method treating seizure clusters in a subject in need thereof comprising administering to the subject a composition comprising an effective amount of a benzodiazepine, wherein administration of the composition increases the length of an interseizure cluster interval (ISCI) in the subject.

Embodiment 47. A method of increasing the time to a seizure cluster in a subject suffering from recurrent seizure clusters comprising administering to the subject a composition comprising an effective amount of a benzodiazepine, wherein administration of the composition increases the length of an ISCI in the subject.

Embodiment 48. The method of embodiments 46 or 47, wherein the composition is an oral, intravenous, rectal, or intranasal formulation.

Embodiment 49. The method of any one of embodiments 46-48, wherein the composition is an intranasal formulation administered to a nasal mucosal membrane of the subject.

Embodiment 50. The method of any one of embodiments 46-49, wherein composition is administered to the subject before, during, or after a seizure within a seizure cluster or acute repetitive seizures.

Embodiment 51. The method of embodiment 50, wherein the seizure is an absence seizure, a myoclonic seizure, a clonic seizure, a tonic seizure, a tonic-clonic seizure, an atonic seizure, a focal seizure, or any combination thereof.

Embodiment 52. The method of any one of embodiments 46-51, wherein the method comprises administering at least two or more doses of the composition comprising an effective amount of a benzodiazepine.

Embodiment 53. The method of embodiment 52, wherein the method comprises administering at least two or more doses of the composition over at least a three month period.

Embodiment 54. The method of any one of embodiments 46-53, wherein the subject has a decrease in the frequency of seizure clusters.

Embodiment 55. The method of any one of embodiments 46-54, wherein the ISCI duration increases by at least 1, 2, 3, 4, 5, 6, 7, 8, 9, 10, 11, 12, 13, 14, 15, 16, 17, 18, 19, or 20 days.

Embodiment 56. The method of any one of embodiments 46-55, wherein the benzodiazepine is alprazolam, brotizolam, chlordiazepoxide, clobazepam, clonazepam, clorazepate, demoxepam, diazepam, estazolam, flurazepam, quazepam, halazepam, lorazepam, midazolam, nitrazepam, nordazapam, oxazepam, prazepam, quazepam, temazepam, triazolam, zolpidem, zaleplon, olanzapine, flumanezil, or combinations thereof.

Embodiment 57. The method of any one of embodiments 46-56, wherein the benzodiazepine is diazepam.

Embodiment 58. The method of any one of embodiments 46-57, wherein the composition comprises an effective amount of diazepam, an alkyl maltoside, and a carrier system comprising one or more natural or synthetic tocopherols or tocotrienols and one or more alcohols.

Embodiment 59. The method of embodiment 58, wherein the effective amount of diazepam is about 5 mg to about 20 mg diazepam in a volume of about 10 μL to 200 μL of the composition.

Embodiment 60. The method of embodiments 58 or 59, wherein the alkyl maltoside is selected from dodecyl maltoside, tetradecyl maltoside, or a combination thereof.

Embodiment 61. The method of any one of embodiments 58-60, wherein the composition comprises about 0.1% w/v to about 1% w/v of the alkyl maltoside.

Embodiment 62. The method of any one of embodiments 58-61, wherein the one or more alcohols comprises a mixture of ethanol and benzyl alcohol.

Embodiment 63. The method of any one of embodiments 58-62, wherein the one or more alcohols comprises benzyl alcohol.

Embodiment 64. The method of any one of embodiments 46-63, wherein the seizure or seizure cluster is caused by epilepsy or an epileptic disorder.

Embodiment 65. The method of any one of embodiments 46-64, wherein the administering is performed by the subject or a caregiver.

Embodiment 66. The method of any one of embodiments 46-64, wherein the administering is performed by the subject.

Embodiment 67. The method of any one of embodiments 46-66, wherein the administering is performed in a prodromal phase or pre-ictal phase of the seizure.

Embodiment 68. The method of any one of embodiments 46-67, wherein the administering is performed in an ictal phase of the seizure.

Embodiment 69. The method of any one of embodiments 46-68, wherein the administering is performed in a post-ictal phase of the seizure.

Embodiment 70. The method of any one of embodiments 46-69, further comprising:
administering a second effective amount of the composition within 4 hours of said administering.

Embodiment 71. A pharmaceutical solution for nasal administration consisting of: diazepam or a pharmaceutically acceptable salt thereof one or more natural or synthetic tocopherolds or tocotrienols, or any combination thereof, in an amount from about 35% to about 45% (w/v); benzyl alcohol in an amount from about 35% to about 45% (w/v); and n-dodecyl beta D-maltoside (DDM) in an amount from about 0.15% to about 0.75% (w/v).

Embodiment 72. The pharmaceutical solution of embodiment 71, wherein the diazepam or a pharmaceutically acceptable salt thereof is present in a concentration of 5 to 20% (w/v).

Embodiment 73. The pharmaceutical solution of embodiment 72, wherein the diazepam or a pharmaceutically acceptable salt thereof is present in a concentration of 20% (w/v).

Embodiment 74. The pharmaceutical solution of embodiment 71, wherein the diazepam or a pharmaceutically acceptable salt thereof is present in a concentration of 100 mg/mL to 500 mg/mL in about 10 μL to 200 μL dose of the pharmaceutical solution.

Embodiment 75. The pharmaceutical solution of embodiment 74, wherein the diazepam or a pharmaceutically acceptable salt thereof is present in a concentration of about 200 mg/mL in about 100 μL of the pharmaceutical solution.

Embodiment 76. The pharmaceutical solution of any one of embodiments 71-75, wherein the one or more natural or synthetic tocopherols or tocotrienols are selected from the group consisting of: α-tocopherol, β-tocopherol, γ-tocopherol, δ-tocopherol, α-tocotrienol, β-tocotrienol, γ-tocotrienol, δ-tocotrienol, tocophersolan, any isomers thereof, any esters thereof, any analogs or derivatives thereof, and any combinations thereof.

Embodiment 77. The pharmaceutical solution of embodiment 76, wherein the one or more natural or synthetic tocopherols or tocotrienols is vitamin E.

Embodiment 78. The pharmaceutical solution of any one of embodiments 71-77, wherein the one or more natural or synthetic tocopherols or tocotrienols is present in an amount from about 40% to about 42% (w/v).

Embodiment 79. The pharmaceutical solution of any one of embodiments 71-78, wherein the benzyl alcohol is present in an amount from about 40% to about 42% (w/v).

Embodiment 80. The pharmaceutical solution of any one of embodiments 71-79, wherein the DDM is present in an amount from about 0.25% to about 0.75% (w/v).

Embodiment 81. The pharmaceutical solution of embodiment 80, wherein the DDM is present in an amount of about 0.25% (w/v).

Embodiment 82. The pharmaceutical solution of embodiment 80, wherein the DDM is present in an amount of about 0.50% (w/v).

Embodiment 83. The pharmaceutical solution of embodiment 80, wherein the DDM is present in an amount of about 0.75% (w/v).

Embodiment 84. The pharmaceutical solution of any one of embodiments 71-83, wherein the nasal administration is a single dose nasal administration.

Embodiment 85. A pharmaceutical solution for a single dose nasal administration consisting of: diazepam or a pharmaceutically acceptable salt thereof in an amount of about 20% (w/v); vitamin E in an amount from about 40% to about 42% (w/v); benzyl alcohol in an amount from about 40% to about 42% (w/v); and n-dodecyl beta D-maltoside (DDM) in an amount of about 0.25% (w/v).

Embodiment 86. A pharmaceutical solution for a single dose nasal administration consisting of: diazepam or a pharmaceutically acceptable salt thereof in an amount of about 20% (w/v); vitamin E in an amount from about 40% to about 42% (w/v); benzyl alcohol in an amount from about 40% to about 42% (w/v); and n-dodecyl beta D-maltoside (DDM) in an amount of about 0.50% (w/v).

Embodiment 87. A pharmaceutical solution for a single dose nasal administration consisting of: diazepam or a pharmaceutically acceptable salt thereof in an amount of about 20% (w/v); vitamin E in an amount from about 40% to about 42% (w/v); benzyl alcohol in an amount from about 40% to about 42% (w/v); and n-dodecyl beta D-maltoside (DDM) in an amount of about 0.75% (w/v).

Embodiment 88. A method of administering diazepam or a pharmaceutically acceptable salt thereof to a subject in need thereof comprising intranasally administering any of the pharmaceutical solutions of embodiments 71-87 to a nasal mucosal membrane of the subject in a single dose.

Embodiment 89. A method of treating a seizure in a subject in need thereof comprising intranasally administering any of the pharmaceutical solutions of embodiments 71-87 to a nasal mucosal membrane of the subject in a single dose.

Embodiment 90. A method of inhibiting the onset of a seizure comprising intranasally administering any of the pharmaceutical solutions of embodiments 71-87 to a nasal mucosal membrane of a subject in a single dose during a prodromal or pre-ictal phase of the seizure.

Embodiment 91. A method of treating recurrent seizures in a subject in need thereof comprising intranasally administering any of the pharmaceutical solutions of embodiments 71-87 to a nasal mucosal membrane of the subject in a single dose during or before a seizure.

Embodiment 92. A method of improving patient compliance with a treatment regimen for preventing or treating a seizure, the method comprising intranasally administering any of the pharmaceutical solutions of embodiments 71-87 to a nasal mucosal membrane of the subject in a single dose.

Embodiment 93. The method of any one of embodiments 88-92, wherein the administering is performed by the subject or a caregiver.

Embodiment 94. The method of any one of embodiments 88-93, wherein the administering is performed by the subject.

Embodiment 95. The method of any one of embodiments 89-93, wherein the seizure is caused by epilepsy or an epileptic disorder.

Embodiment 96. The method of any one of embodiments 89-93, wherein the seizure is an absence seizure, a myoclonic seizure, a clonic seizure, a tonic seizure, a tonic-clonic seizure, an atonic seizure, a focal seizure, or any combination thereof.

Embodiment 97. The method of any one of embodiments 89-92, wherein the seizure is a seizure within a seizure cluster or acute repetitive seizures.

### EXAMPLES

EXAMPLE 1: Formulations—Various non-limiting examples of intranasal compositions comprising a benzodiazepine, as described herein, are provided in Table 1 below. In any embodiment, the alkyl maltoside may be tetradecyl maltoside or dodecyl maltoside, preferably dodecyl maltoside. In any embodiment, the Vitamin E may be, for example, α-tocopherol, β-tocopherol, γ-tocopherol, δ-tocopherol, α-tocotrienol, β-tocotrienol, γ-tocotrienol, δ-tocotrienol, tocophersolan, preferably α-tocopherol.

TABLE 1

| | Solution No. | | | | | |
|---|---|---|---|---|---|---|
| Component | 1 | 3 | 4 | 6 | 7 | 8 |
| diazepam (% w/v) | 5-15 | 9-11 | 10 | 5 | 7.5 | 5-20 |
| alkyl maltoside (% w/v) | 0.01-1 | 0.1-0.5 | 0.15-0.3 | 0.01-1 | 0.01-1 | 0.01-1 |
| vitamin E (% w/v) | 45-65 | 50-60 | 50-60 | 70 | 65 | 35-45 |
| ethanol (% w/v) | 10-25 | 15-22.5 | 17-20 | q.s. | q.s. | 0 |
| benzyl alcohol (% w/v) | 5-15 | 7.5-12.5 | 10-12 | — | — | 35-45 |

EXAMPLE 2: Rapid Onset of Therapeutic Effect—43 Subjects (24 female/19 male aged 6-59) were administered 10 mg, 15 mg, or 20 mg of diazepam via an intranasal composition, as disclosed herein, intranasally, using one of the compositions as depicted in Table 2 below. For subjects receiving 15 mg, 100 μL of the 75 mg/mL diazepam solution was administered to each nostril. Likewise, for subject receiving 20 mg, 100 μL of the 10 mg/mL diazepam solution was administered to each nostril. A 10 mg dose was administered as 100 μL of the 10 mg/mL diazepam solution to a single nostril.

TABLE 2

| Component | 50 mg/mL | | 75 mg/mL | | 100 mg/mL | |
|---|---|---|---|---|---|---|
| Diazepam | 5.00 | mg | 7.50 | mg | 10.0 | mg |
| Vitamin E | 56.47 | mg | 56.47 | mg | 56.47 | mg |
| Dodecyl maltoside | 0.25 | mg | 0.25 | mg | 0.25 | mg |
| Benzyl alcohol | 10.50 | mg | 10.50 | mg | 10.50 | mg |

| | | | |
|---|---|---|---|
| Dehydrated ethanol | q.s. to 100 μL | q.s. to 100 μL | q.s. to 100 μL |

Administration of diazepam to the subject via an intranasal composition occurred in the during an active seizure in 16 of the subjects. Cessation of the seizure, as measured by EEG, was achieved within about 1 minute in all but one of the subjects. Table 3 below depicts the rapid therapeutic effect gleaned from administration of the composition in each of the subjects tested (a=am, p=pm).

TABLE 3

| | | | | | | | | | Time to |
|---|---|---|---|---|---|---|---|---|---|
| | | | | | Seizure | | Seizure | | Seizure |
| | | | Time of | | Start | | Stop | | Cessation |
| Gender | Age | Dosage | Dosing | | Time | | Time | | (min) |
| F | 32 | 20 mg | 5:55 | p | 5:55 | p | 5:55 | p | <1 |
| M | 38 | 20 mg | 9:10 | a | 9:10 | a | 9:10 | a | <1 |
| M | 24 | 15 mg | 9:02 | a | 9:02 | a | 9:02 | a | <1 |
| F | 34 | 20 mg | 8:10 | a | 8:10 | a | 8:10 | a | <1 |
| F | 47 | 20 mg | 12:46 | p | 12:45 | p | 12:46 | p | <1 |
| M | 34 | 20 mg | 9:49 | a | 9:46 | a | 9:49 | a | <1 |
| F | 45 | 15 mg | 4:57 | p | 4:57 | p | 4:57 | p | <1 |
| F | 39 | 15 mg | 7:03 | p | 7:03 | p | 7:04 | p | 1 |
| M | 46 | 20 mg | 12:30 | p | 12:30 | p | 12:30 | p | <1 |
| M | 37 | 20 mg | 5:21 | p | 5:19 | p | 5:22 | p | 1 |
| F | 15 | 15 mg | 1:00 | a | 12:59 | a | 1:02 | a | 2 |
| F | 35 | 15 mg | 4:17 | p | 4:06 | p | 4:17 | p | <1 |
| F | 46 | 20 mg | 6:52 | p | 6:52 | p | 6:52 | p | <1 |
| F | 7 | 10 mg | 1:25 | p | 1:25 | p | 1:26 | p | 1 |
| M | 10 | 15 mg | 6:40 | a | 6:39 | a | 6:40 | a | <1 |
| F | 12 | 15 mg | 6:24 | p | 6:23 | p | 6:25 | p | 1 |
| F | 45 | 20 mg | 10:06 | a | 10:06 | a | 10:06 | a | <1 |
| F | 20 | 10 mg | 9:13 | a | 9:13 | a | 9:13 | a | <1 |
| F | 46 | 20 mg | 1:23 | p | 1:23 | p | 1:23 | p | <1 |
| M | 31 | 20 mg | 9:16 | a | 9:16 | a | 9:16 | a | <1 |
| F | 42 | 15 mg | 10:32 | a | 10:32 | a | 10:32 | a | <1 |
| F | 36 | 20 mg | 10:41 | a | 10:41 | a | 10:41 | a | <1 |
| F | 57 | 20 mg | 9:57 | a | 9:57 | a | 9:57 | a | <1 |
| F | 24 | 15 mg | 8:19 | a | 8:19 | a | 8:19 | a | <1 |
| F | 13 | 10 mg | 9:17 | a | 9:17 | a | 9:17 | a | <1 |

The intranasal compositions as disclosed herein may be used to prevent future seizures, for example, within a cluster of seizures. Therefore, in some subjects, administration of diazepam to the subject via the intranasal composition occurred after the seizure had already ended, as measured by EEG. In each of these cases, no subsequent seizure was experienced by any of the subjects during the period of observation. Table 4 below depicts the rapid therapeutic effect gleaned from administration of the composition in each of the subjects tested (a=am, p=pm).

TABLE 4

| | | | | | Seizure | | Seizure | | Sub- |
|---|---|---|---|---|---|---|---|---|---|
| | | | Time of | | Start | | Stop | | sequent |
| Gender | Age | Dosage | Dosing | | Time | | Time | | Seizure |
| M | 31 | 20 mg | 8:26 | a | 8:25 | a | 8:25 | a | N |
| M | 42 | 20 mg | 7:10 | p | 7:06 | p | 7:08 | p | N |
| F | 59 | 20 mg | 11:06 | a | 11:05 | a | 11:05 | a | N |
| F | 32 | 15 mg | 7:57 | a | 7:54 | a | 7:56 | a | N |
| M | 37 | 20 mg | 10:25 | a | 10:22 | a | 10:22 | a | N |
| M | 59 | 20 mg | 11:18 | a | 11:15 | a | 11:16 | a | N |
| F | 33 | 15 mg | 12:50 | p | 12:45 | p | 12:47 | p | N |
| M | 31 | 20 mg | 3:35 | p | 3:20 | p | 3:25 | p | N |
| M | 30 | 20 mg | 11:47 | p | 11:46 | p | 11:46 | p | N |
| F | 18 | 15 mg | 7:58 | p | 7:57 | p | 7:57 | p | N |
| M | 15 | 10 mg | 8:05 | p | 7:50 | p | 7:58 | p | N |
| M | 19 | 15 mg | 12:44 | p | 12:42 | p | 12:43 | p | N |
| M | 9 | 20 mg | 12:40 | p | 12:37 | p | 12:39 | p | N |
| F | 9 | 15 mg | 12:47 | p | 12:45 | p | 12:46 | p | N |
| M | 6 | 10 mg | 12:20 | p | 12:18 | p | 12:19 | p | N |
| M | 21 | 10 mg | 11:10 | a | 11:07 | a | 11:08 | a | N |
| M | 34 | 20 mg | 5:53 | p | 5:48 | p | 5:48 | p | N |
| F | 11 | 10 mg | 9:48 | a | 9:43 | a | 9:43 | a | N |

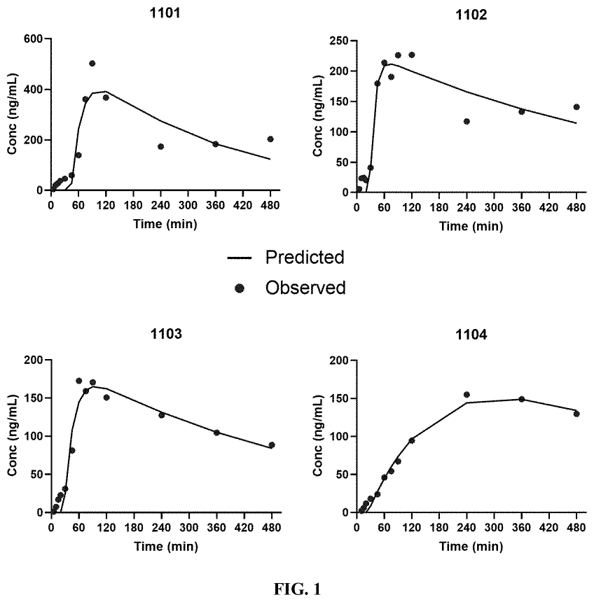

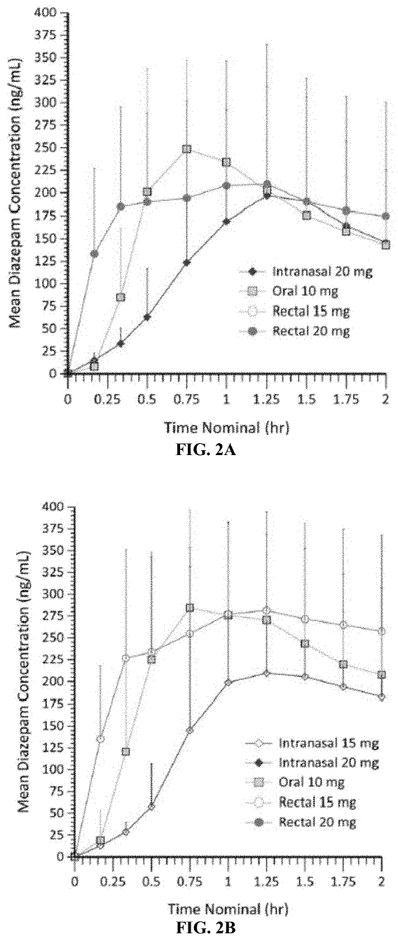

EXAMPLE 3: PK Data—Timing of Maximum Plasma Concentrations: Four subjects were administered 20 mg diazepam intranasally formulated according to Example 1. FIG. 1 depicts the plasma concentrations versus time for each subject, illustrating that despite near-immediate therapeutic benefit post-administration, Tₘₐₓ is not achieved until at least an hour after administration. FIG. 2A and FIG. 2B depict plasma concentrations of diazepam administered orally (10 mg), rectally (15 mg or 20 mg), and intranasally (15 mg or 20 mg). Notably, a later Tₘₐₓ is achieved in compositions administered intranasally.

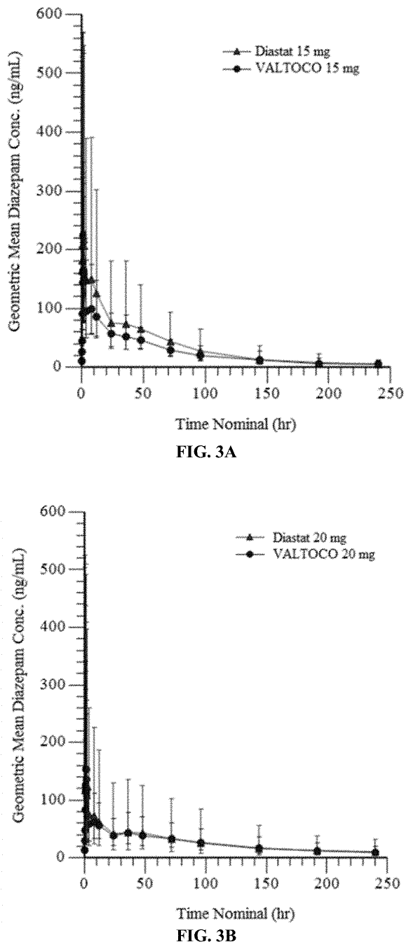

EXAMPLE 4: PK Data—Bioavailability/AUC: Twenty-eight subjects received 15 mg or 20 mg diazepam administered intranasally using the described in Example 1. Forty-three (43) subjects were administered 15 mg or 20 mg diazepam rectally (DIASTAT®). As shown in FIG. 3A and FIG. 3B, compared to rectally administered diazepam, the intranasally administered diazepam resulted in comparable AUC values at the 20 mg dose and slightly lower at the 15 mg dose. While not wishing to be bound by theory, the slightly lower values at the 15 mg dose may be attributed to variability in DIASTAT® dosing.

EXAMPLE 5: Clinical Study on Adverse Effects: A study of adverse effects (AE) was conducted over a one-year time period spanning 163 subjects and 3853 seizure clusters. After four months of treatment of frequent or breakthrough seizures with the diazepam formulated as described in Example 1, 134 of the 163 subjects reported at least one AE, of which the AEs of only 30 of the 134 subjects were considered treatment-related. None of the treatment-related AEs were serious. Subjects reported AEs considered treatment-related such as nasal discomfort (10 subjects), headache (4 subjects), epistaxis (3 subjects), dysgeusia (3 subjects), somnolence (3 subjects), cough (2 subjects), rhinalgia (2 subjects), rhinorrhea (2 subjects), migraine (2 subjects), fatigue (2 subjects), eye irritation (2 subjects), and lacrimation (2 subjects) increase. Nasal mucus disorder, nasal pruritus, rash, throat irritation, tonsillar hypertrophy, lethargy, loss of consciousness, sensory integrative dysfunction, sedation, ocular hyperemia, nausea, retching, insomnia, sleep disorder, and administration site pain were each reported by a single subject. There were no incidents of respiratory depression. Overall, the safety study demonstrated that diazepam administered intranasally according to the methods disclosed herein is safe and well tolerated in subjects with epilepsy who have frequent breakthrough seizures.

EXAMPLE 6: Clinical Study on AEs: A dose of 5 mg, 10 mg, 15 mg, or 20 mg of diazepam according to the intranasal compositions described in Example 1 was selected according to the subject's weight (rounded to the nearest kg). Children aged 6-11 years and 10 kg to 18 kg body weight received a 5 mg dose (50 mg/milliliter [mL], 100 microliters [μL]) administered as one spray in the left nostril; 19 kg to 37 kg body weight, received a 10 mg dose (100 mg/mL, 100 μL) administered as one spray in the left nostril; 38 kg to 55 kg body weight received a 15 mg dose (75 mg/mL, 100 μL) administered as two 7.5 mg sprays with one in each nostril (the left nostril will be sprayed first followed by the right nostril); and 56 kg to 74 kg body weight, received a 20 mg dose (100 mg/mL, 100 μL) of diazepam administered as two 10 mg sprays with one in each nostril (the left nostril will be sprayed first followed by the right nostril). Subjects 12 years or greater and 14 kg to 27 kg body weight received a 5 mg dose (50 mg/mL, 100 μL) administered as one spray in the left nostril; 28 kg to 50 kg body weight received a 10 mg dose (100 mg/mL, 100 μL) administered as one spray in the left nostril; 51 kg to 75 kg body weight received a 15 mg dose (75 mg/mL, 100 μL) administered as two 7.5 mg sprays with one in each nostril (the left nostril will be sprayed first followed by the right nostril); and greater than 76 kg body weight received a 20 mg dose (100 mg/mL, 100 μL) of diazepam administered as two 10 mg sprays with one in each nostril (the left nostril was sprayed first followed by the right nostril). A total of 4015 seizures were treated with diazepam across 175 epilepsy patients. A total of 163 of the 175 enrolled subjects (93.1%) received study drug and were included in a safety population. Of the 163 subjects included in the safety population, 45 subjects were between the ages of 6 and 11 years old. Of the 45 subjects, 3 (6.7%) were treated for less than 6 months, 7 (15.6%) were treated for 6 to less than 12 months, and 35 (77.8%) were treated for 12 months or longer. Eight subjects (17.8%) received 1 to 2 doses, 12 subjects (26.7%) received 3 to 10 doses, 9 subjects (20.0%) received 11 to 20 doses, 10 subjects (22.2%) received 21 to 40 doses, and 6 subjects (13.3%) received more than 40 doses. There were 19 subjects (42.2%) in this 6-11-year-old age group that averaged 2 or more doses per month. Of the 163 subjects included in the safety population, 118 subjects were 12 years of age or older. Of the 118 subjects, 6 (5.1%) were treated for less than 6 months, 14 (11.9%) were treated for 6 to less than 12 months, and 98 (83.1%) were treated for 12 months or longer. Fifteen (15) subjects (12.7%) received 1 to 2 doses, 30 subjects (25.4%) received 3 to 10 doses, 23 subjects (19.5%) received 11 to 20 doses, 25 subjects (21.2%) received 21 to 40 doses, and 25 subjects (21.2%) received more than 40 doses. There were 67 subjects (56.8%) in this age group (>12 years old) that averaged 2 or more doses per month.

Of the 163 subjects included in the safety population, 27 subjects self-administered the intranasal compositions described in Example 1. Of the 27 subjects, none were treated for less than 6 months, 1 (3.7%) was treated for 6 to less than 12 months, and 26 (96.3%) were treated for 12 months or longer. Three (3) subjects (11.1%) received 1 to 2 doses, 2 subjects (7.4%) received between 3 to 10 doses, 6 subjects (22.2%) received between 11 to 20, 8 subjects (29.6%) received between 21 to 40 doses, and 8 subjects (29.6%) received more than 40 doses. There were 19 subjects (70.4%) in this self-administrator group that averaged 2 or more doses per month.

The 163 subjects in the Safety population experienced 3853 seizure clusters. Of the 3853 seizure clusters, 485 (12.6%) required a second dose of medication. Overall, 134 out of 163 subjects (82.2%) had at least one AE. Thirty (30) subjects (18.4%) had AEs that were considered treatment-related. None of the AEs that were considered treatment-related were serious. The treatment-related AEs were: nasal discomfort (10 subjects, 6.1%); headache (4 subjects, 2.5%); epistaxis, dysgeusia, somnolence (3 subjects each, 1.8%); cough, rhinalgia, and rhinorrhea, migraine, eye irritation, lacrimation increased, fatigue (2 subjects each, 1.2%), nasal mucosal disorder, nasal pruritus, throat irritation, tonsillar hypertrophy, lethargy, loss of consciousness, sensory integrative dysfunction, ocular hyperemia, administration site pain, nausea, retching, insomnia, sleep disorder, and rash (1 subject each, 0.6%). Overall, diazepam, administering via the intranasal compositions according to Example 1, demonstrated a good safety profile for treating seizures.

Notably, of the 175 subjects enrolled, 27 of the subjects were self-administrators and able to administer the composition prior to onset of seizure due to prodromal or pre-ictal symptoms, such as aura. The seizures experienced by the self-administrators accounted for over 900 of the 4390 doses administered using the intranasal compositions as described in Example 1.

EXAMPLE 7: Clinical Study on PK Profile: Twenty-four subjects were randomized. All 24 subjects received a dose of diazepam in one of three ways: diazepam nasal spray (suspension, 10 mg), diazepam nasal spray (solution, 10 mg), or diazepam intravenous (5 mg/mL for 1 minute). Fourteen days later, the subject returned for administration of diazepam via a different dosing than the first dosing. After another 14 days, subject was administered a final dose of diazepam, different than the first two, having been administered diazepam in all three manners throughout the trial period. Table 5 below reports various PK measurements made for each dose type.

TABLE 5

| | Diazepam Nasal Spray (10 mg/100 μL) | | | | | | Diazepam Injection | | |
|---|---|---|---|---|---|---|---|---|---|
| | NRL-1.A Suspension | | | NRL-1.B Solution | | | 5 mg/mL IV | | |
| Parameter^{a} | n | Mean (SD)^{b} | | n | Mean (SD)^{b} | | n | Mean (SD)^{b} | |
| Cₘₐₓ (ng/mL) | 24 | 221 | (78.6) | 24 | 272 | (100) | 24 | 555 | (316) |
| Tₘₐₓ (h) | 24 | 1.00 | (0.6, 2.0) | 24 | 1.50 | (0.8, 4.0) | 24 | 0.03 | (0.03, 0.50) |
| AUC₀₋ₜ (h × ng/mL) | 24 | 5229 | (1463) | 24 | 7340 | (1882) | 24 | 3832 | (1150) |
| AUC_{0-∞} (h × ng/mL) | 20 | 5381 | (1409) | 20 | 7338 | (2072) | 24 | 4104 | (1318) |
| λz (b⁻¹) | 20 | 0.0142 | (0.0053) | 20 | 0.0155 | (0.0046) | 24 | 0.0142 | (0.0055) |
| t½ (h) | 20 | 56.2 | (23.0) | 20 | 49.2 | (16.9) | 24 | 56.2 | (21.0) |

^{a}Mean values are presented as arithmetic means
^{b}Median (min, max) reported for Tₘₐₓ

Differences between the solution and suspension with respect to the metabolite, desmethyldiazepam, were consistent with those for the intravenous injection. Metabolite-to parent ratios suggest little or no contribution to the extent of formation of desmethyldiazepam by first-pass metabolism and thus a low likelihood that any of the intranasal-administered diazepam was absorbed from the gastrointestinal tract after swallowing any “run off” from the back of the nose.

Safety results show that the intranasal administration of diazepam via suspension and solution are well-tolerated. Most subjects experienced at least one AE, most frequently epistaxis (7 subjects) and somnolence (6 subjects). Somnolence, however was more commonly associated with IV diazepam (4 subjects) than either of the intranasal dosages (1 subject for each). Reports of epistaxis was also higher following diazepam administered intravenously (5 events) than after diazepam administered intranasally via the intranasal compositions as disclosed herein (3 events).

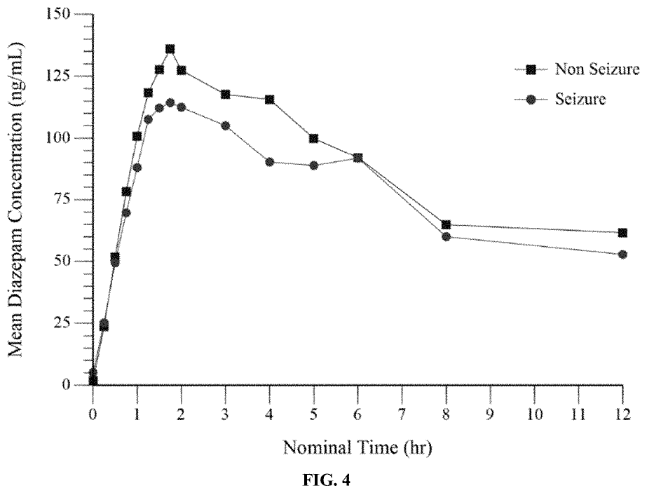

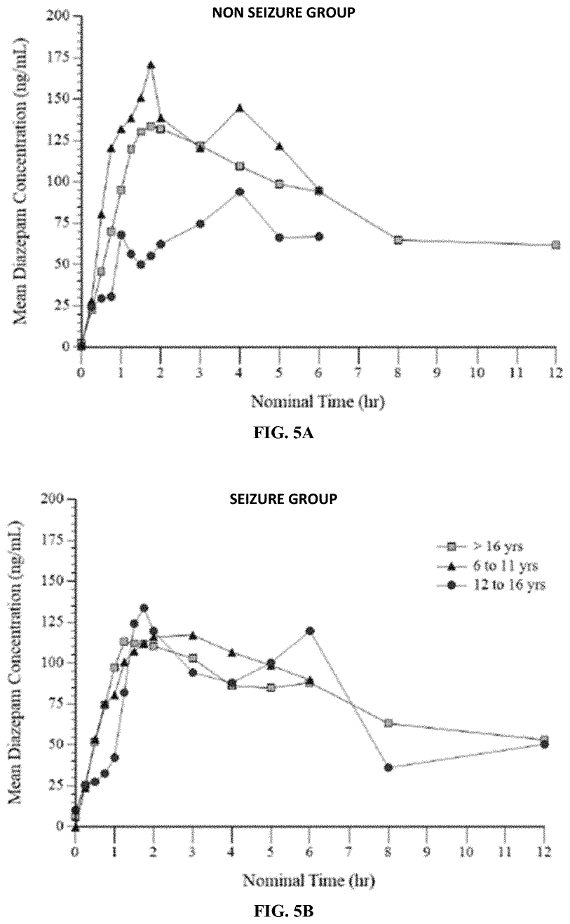

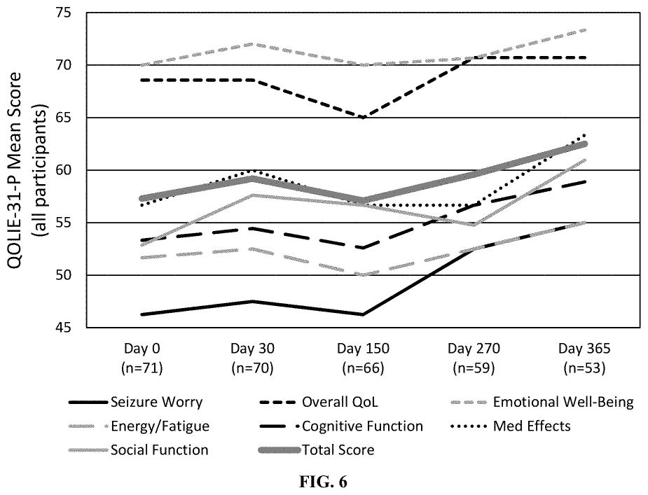

EXAMPLE 8: Clinical Study on AEs & PK Data: Fifty-seven (57) subjects were administered 5 mg, 10 mg, 15 mg, or 20 mg diazepam intranasally according to the compositions disclosed in Example 1, based on the subject's body weight, during an ictal, peri-ictal, or interictal (non-seizing) phase. As shown in FIG. 4, FIG. 5A, and FIG. 5B, various aspects of the PK profile of diazepam administered via an intranasal composition, as disclosed herein, in subjects suffering from epilepsy was not notably altered by their physiological condition (e.g., seizure or non-seizure state) for any of the age groups or at any dose evaluated.

All fifty-seven (57) subjects were included in the Safety Population. Three (3) subjects (5.3%) withdrew from the study prior to receiving diazepam nasal spray. Per protocol, each subject was to receive two doses of diazepam via administered via an intranasal composition described in Example 1. 10 mg, 15 mg, or 20 mg dosages were administered, based on the subject's body weight. Thirteen (13) subjects were in the 10 mg dose group, 19 subjects were in the 15 mg dose group, and 25 subjects were in the 20 mg dose group. Overall, 17 out of 57 subjects (29.8%) experienced at least one AE. Eight (8) subjects (14.0%) had AEs that were considered treatment-related. Overall, the most common treatment-emergent AEs (TEAEs) were dysgeusia (3 subjects, 5.3%), seizure, nasopharyngitis, and nasal discomfort (2 subject each, 3.5%). There were no dose-related trends or changes in nasal irritation from pre-dose to dosing to post dose. No irritation was observed for the majority of the subjects at any time point after administration, and for those that reported irritation, only focal nasal mucosal irritation or inflammation was reported. Three subjects had suicidal ideation or behavior prior to dosing. No subjects had suicidal ideation at any time post-dosing.

EXAMPLE 9: Quality of Life in Epilepsy Scale from a Long-Term Safety Study of Diazepam Nasal Spray for Seizure Cluster. The burden of seizure clusters affects patient quality of life (QoL), but few studies have examined this impact. Diazepam nasal spray is approved for acute treatment of seizure clusters in patients with epilepsy aged ≥6 years. The present example provides a long-term safety study of diazepam nasal spray examines results of the patient-reported Quality of Life in Epilepsy (QOLIE) questionnaire among adults with seizure clusters.

Patients aged 6-65 years in a long-term, open-label safety study of diazepam nasal spray (Valtoco®) used 5-, 10-, 15- or 20-mg doses (based on age and weight) to treat seizure clusters. QOLIE-31-P is an epilepsy-specific instrument to assess health-related QoL in adult patients. The total score, on a 100-point scale, is a weighted composite of 7 separate 100-point subscales: Seizure Worry, Overall QoL, Emotional Well-Being, Energy/Fatigue, Cognitive Functioning, Medication Effects, and Social Functioning. Higher scores indicate better QoL. Patients ≥18 years old completed the QOLIE-31-P at baseline (day 0) and days 30, 150, 270, and 365, recording responses covering the prior 4 weeks. Descriptive statistics were calculated across time points using all available data from each patient (All Data) and data from the subset of patients who completed QOLIE-31-P at all 5 time points (Completers).

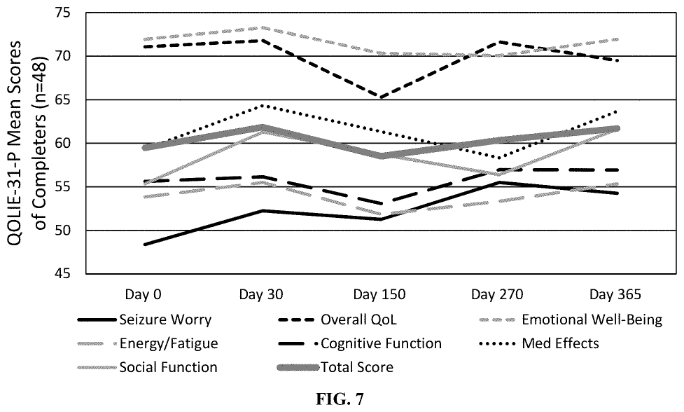

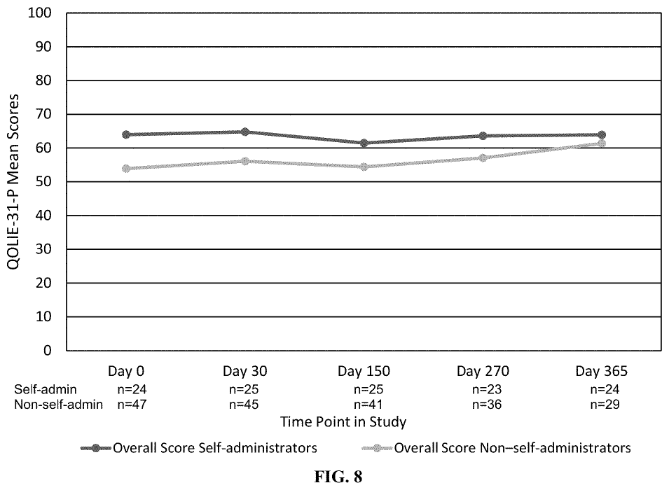

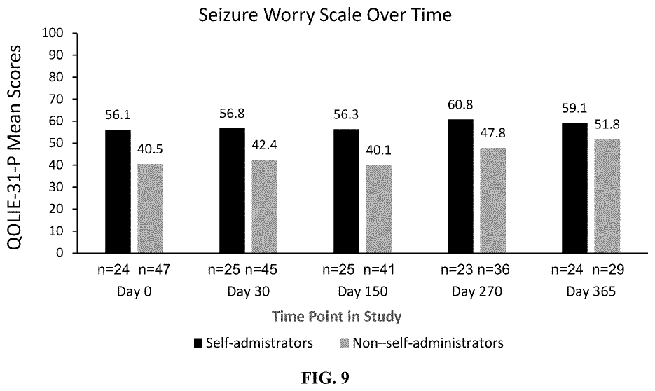

Among 163 treated patients, 74 adults (59.5% female) provided data and had a mean age of 35.6 years. Exposure was ≥12 months for 60 patients (range, 3.1-40.4 months), with a mean of 2.4 doses per month. Mean QOLIE-31-P scores tended to be similar or increase slightly across time points, with maximum at day 365 in both the All Data (n=53-71; FIG. 6) and Completer (n=48; FIG. 7) analyses. Sub score means for Seizure Worry and Social Functioning showed the greatest numeric increase from baseline. The mean change from day 0 to day 365 for Seizure Worry was 8.75 (All Data) and 5.88 (Completers); and for Social Functioning was 8.10 (All Data) and 6.29 (Completers). Total scores increased from day 0 to day 365 by 5.20 (All Data) and 2.21 (Completers).

Tolerability was similar to that seen in a full study cohort with 23.0% reporting treatment-related TEAEs; none of which were serious or led to discontinuation.

In this analysis, adults with highly refractory epilepsy treated with diazepam nasal spray for seizure clusters maintained QOLIE scores over the course of the 12-month study period, generally with small numeric improvement. Seizure Worry and Social Functioning subscale scores showed apparent improvements over time. Differences in results for individual subscales may suggest variations in sensitivity to use of an intermittent treatment. Availability of diazepam nasal spray for out-of-hospital seizure treatment has potential to improve patients' sense of empowerment to treat seizure clusters.

EXAMPLE 10: Adults Self-Administering Diazepam Nasal Spray for Seizure Clusters. Seizure clusters negatively impact quality of life (QoL). Within a subgroup of patients in a phase 3 study self-administered diazepam nasal spray, 78% found it easy to use and 48% primarily administered it at the first sign a seizure may be coming. This analysis of the self-administering adults in that study examines results of the Quality of Life in Epilepsy (QOLIE) questionnaire.

Age- and weight-based doses of diazepam nasal spray were used in this phase 3, long-term, repeat-dose safety study in patients aged 6-65 years. The QOLIE-31-P, a self-reported tool for adults (≥18 years), uses a 4-week recall period and was given at days 0 (baseline), 30, 150, 270, and 365. The overall score is a weighted composite of 7 subscales: Seizure Worry, Overall QoL, Emotional Well-Being, Energy/Fatigue, Cognitive Functioning, Medication Effects, and Social Functioning; scores were analyzed for adults who self-administered diazepam nasal spray, with higher scores indicating better QoL. Descriptive statistics were calculated, and the 2-sided, 2-sample t test was used for comparison with non-self-administering adults.

Of 163 treated patients, 27 (16.6%) self-administered diazepam nasal spray (25 adults); 24 (96.0%) completed the last study visit at day 365. Duration of use of diazepam nasal spray was ≥12 months for 96.3% (range, 7.4-39.7 months), with a mean of 2.8 doses per patient per month and 1057 total doses overall.

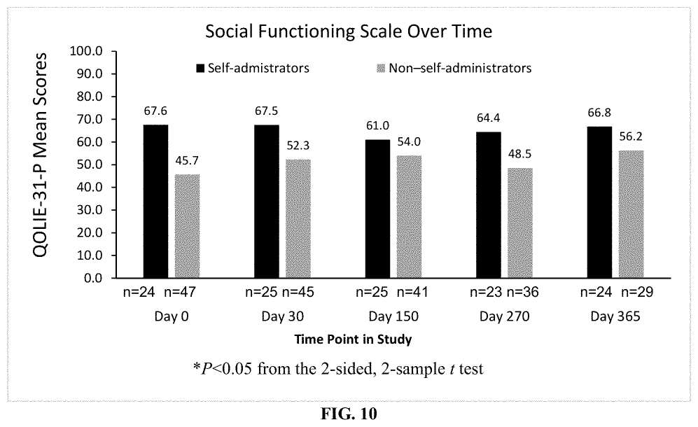

QOLIE-31-P was given to the 25 adult self-administrators and 30 other adults. Overall QOLIE scores for self-administrators were maintained and similar to baseline at all-time points (FIG. 8). Among unadjusted subscales, mean scores for seizure worry were higher for self-administrators than scores for other adults by 7.3-16.2 points through day 365 (FIG. 9); for social functioning, mean scores were higher for self-administrators by 7.0-21.9 points (FIG. 10).

TEAEs were reported in 76.0% (19/25) of adult self-administrators, with treatment-related TEAEs reported in 32.0% (8/25). Serious TEAEs were reported in 6 patients; none were treatment related.

The QOLIE scores reported here, especially those for the Seizure Worry and Social Functioning subscales, suggest that the 25 adult patients able to self-administer diazepam nasal spray in this study may have had greater functional status than the non-self-administering adults. The potential for self-administration represents a possible benefit of diazepam nasal spray, and self-administrators appeared to maintain QOLIE status from baseline through study end.

EXAMPLE 11: Safety and Time to Second Doses in Age Subgroups of Patients with Seizure Clusters Treated with Diazepam Nasal Spray. Non-intravenous benzodiazepines are key rescue therapies for treatment of outpatient seizure clusters in patients with epilepsy. This analysis assessed safety and time to second doses (as a proxy for effectiveness) in patients with epilepsy aged 6-11 years and 12-65 years from a long-term, phase 3, open-label, repeat-dose safety study of diazepam nasal spray.

The study enrolled patients aged 6-65 years with frequent seizure clusters. Patients and care partners were trained to administer doses of 5, 10, 15, or 20 mg of diazepam nasal spray based on age (≤11 or ≥12 years) and weight, with instructions to administer second doses 4-12 hours later if needed. Investigators could adjust doses for effectiveness or safety reasons. Seizures, drug administration, and TEAEs were recorded in seizure diaries. Use of second doses at 4, 6, 12, and 24 hours was assessed for patients aged 6-11 and ≥12 years.

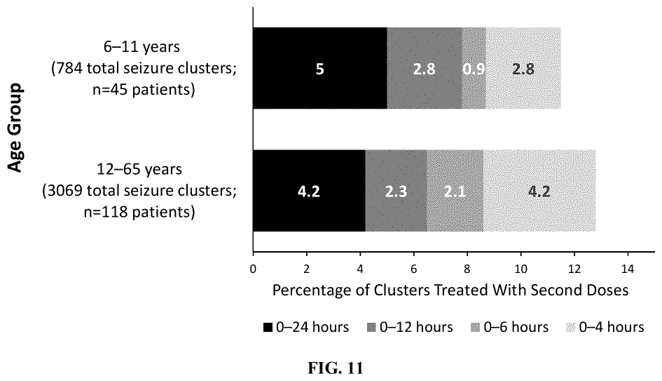

The study enrolled 175 patients, with 163 treated and included in the safety population. Mean age was 23.1 years (6-11 years, n=45 [27.6%]; 12-65 years, n=118 [72.4%]). As shown in FIG. 11, overall mean duration of exposure was 17.4 months and was ≥12 months in 35 (77.8%) of the 6-11 group and 98 (83.1%) of the ≥12 group. The 6-11 group had 784 total seizure clusters, with second doses used in 22 clusters within 4 hours, 7 in 4-6 hours, 22 in 6-12 hours, and 39 in 12-24 hours, for a total of 90 (11.5%) second doses. The ≥12 group had 3069 total seizure clusters, with second doses used in 130 clusters within 4 hours, 65 in 4-6 hours, 234 in 6-12 hours, and 105 in 12-24 hours, for a total of 395 (12.9%) second doses.

TEAEs were reported in 91.1% of patients in the 6-11 group, including 40.0% with serious TEAEs and 6.7% with treatment-related TEAEs (see Table 6). In the ≥12 group, TEAEs were reported for 78.8% of patients, including 27.1% with serious TEAEs and 22.9% with treatment-related TEAEs. None of the serious TEAEs in either group were considered treatment related. One patient in the ≥12 group discontinued owing to a TEAE, and one died; neither was deemed treatment related. The overall completion rate was 71.8%; 7 patients discontinued owing to study closure. Aside from discontinuations due to study closure, the retention rate was 75.5% for the 6-11 group and 76.3% for the ≥12 group.

TABLE 6
TEAEs in Age Subgroups

| | Age 6-11 years | | Age ≥12 years | |
|---|---|---|---|---|
| Patients With TEAEs, n (%) | (n = 45) | | (n = 118) | |
| ≥1 TEAE | 41 | (91.1) | 93 | (78.8) |
| TEAE leading to discontinuation | 0 | | 1 | (0.8)^{a} |
| Serious TEAE | 18 | (40.0) | 32 | (27.1) |

| Treatment-related | 0 | 0 | |
|---|---|---|---|
| Death | 0 | 1 | (0.8)^{a} |

Most common TEAEs (≥5% of either group)

| Nasopharyngitis | 12 | (26.7) | 8 | (6.8) |
|---|---|---|---|---|
| Seizure | 12 | (26.7) | 19 | (16.1) |
| Pyrexia | 10 | (22.2) | 7 | (5.9) |
| Upper respiratory tract infection | 10 | (22.2) | 10 | (8.5) |
| Constipation | 8 | (17.8) | 0 | |
| Influenza | 8 | (17.8) | 5 | (4.2) |
| Pneumonia | 6 | (13.3) | 6 | (5.1) |
| Urinary tract infection | 6 | (13.3) | 5 | (4.2) |
| Vomiting | 6 | (13.3) | 3 | (2.5) |
| Diarrhea | 5 | (11.1) | 4 | (3.4) |
| Sinusitis | 5 | (11.1) | 3 | (2.5) |
| Status epilepticus | 5 | (11.1) | 2 | (1.7) |
| Nausea | 3 | (6.7) | 5 | (4.2) |
| Somnolence | 3 | (6.7) | 8 | (6.8) |
| Contusion | 0 | | 6 | (5.1) |
| Dizziness | 0 | | 8 | (6.8) |
| Headache | 0 | | 7 | (5.9) |
| Nasal discomfort | 0 | | 10 | (8.5) |
| Treatment-related TEAEs | 3 | (6.7) | 27 | (22.9) |

Most common treatment-related TEAEs (>2% in either group)

| | | | | |
|---|---|---|---|---|
| Epistaxis | 1 | (2.2) | 2 | (1.7) |
| Eye irritation | 1 | (2.2) | 1 | (0.8) |

| | | | |
|---|---|---|---|
| Tonsillar hypertrophy | 1 | (2.2) | 0 |

| | | | |
|---|---|---|---|
| Dysgeusia | 0 | 3 | (2.5) |
| Headache | 0 | 4 | (3.4) |
| Nasal discomfort | 0 | 10 | (8.5) |
| Somnolence | 0 | 3 | (2.5) |

^{a}Not deemed treatment related by the investigator.

In an analysis of age subgroups of patients with seizure clusters from a long-term safety study of diazepam nasal spray, use of second doses, safety, and retention rate were similar independent of subgroup. Second doses were administered within 24 hours for 11.5% of seizure clusters among patients 6-11 years and 12.9% among patients ≥12 years. The safety profiles were consistent with the established profile of rectal diazepam. The retention rate was ˜76% in both subgroups.

EXAMPLE 12: Evaluating Tolerability Endpoints Relevant to Clinicians and Patients. Diazepam nasal spray (Valtoco) provides a rapid and noninvasive route of diazepam administration and has been shown to have a safety profile consistent with rectal diazepam across age groups. This analysis of final data from a long-term, phase 3 safety study of diazepam nasal spray follows up on a previously presented analysis from an earlier study to discuss AEs relevant to clinicians and patients.

Individuals aged 6-65 years with a clinical diagnosis of epilepsy experiencing seizure clusters were eligible. Care partners and patients were trained to administer diazepam nasal spray age- and weight-based doses of 5, 10, 15, or 20 mg. A second dose could be administered 4-12 hours later, if needed. Safety assessments included TEAEs, vital signs, and electrocardiograms. Other safety/tolerability outcomes included nasal assessments by a trained observer and olfactory changes on the NIH Toolbox Odor Identification Test.

A total of 175 patients were enrolled, and 163 (54.6% female; mean age, 23.1 years) received ≥1 dose of diazepam nasal spray. Overall, TEAEs were reported for 134 (82.2%) patients; 1 patient had a TEAE (0.6%) resulting in discontinuation that was not treatment related. There were 50 (30.7%) patients with serious TEAEs, none of which were treatment related; one serious TEAE resulted in death. TEAEs considered treatment related were reported in 30 (18.4%) patients; those in >2% of patients were nasal discomfort (6.1%) and headache (2.5%), which were mild or moderate. Rates of cardiorespiratory TEAEs were low (<5% of patients). There were no reported TEAEs of respiratory depression, cardiac depression, or hypotension. Somnolence was reported in 11 (6.7%) patients and sedation in 2 (1.2%) patients. There were no clinically significant abnormalities noted for vital signs; specifically, there were no changes in respiratory rate, blood pressure, or pulse rate with diazepam nasal spray administration. Nasal irritation was uncommon, mild, and transient, as shown in Table 7. In Table 7, the assessment scale was as follows: Grade 0: no sign of nasal irritation or mucosal erosion; grade 1A, focal nasal mucosal irritation or inflammation; grade 1B, superficial mucosal erosion; grade 2, moderate mucosal erosion; grade 3, ulceration; grade 4, septal perforation.

TABLE 7
Nasal Irritation (n = 163)^{a}

| | |
|---|---|
| Time Point | n (%) |

Baseline

| | | |
|---|---|---|
| Grade 0 | 112 | (68.7) |
| Grade 1A | 4 | (2.5) |

Day 30

| | | |
|---|---|---|
| Grade 0 | 113 | (69.3) |
| Grade 1A | 2 | (1.2) |

Day 90

| | | |
|---|---|---|
| Grade 0 | 109 | (66.9) |
| Grade 1A | 2 | (1.2) |

Day 150

| | | |
|---|---|---|
| Grade 0 | 101 | (62.0) |
| Grade 1A | 1 | (0.6) |
| Grade 1B | 1 | (0.6) |

Day 210

| | | |
|---|---|---|
| Grade 0 | 99 | (60.7) |

Day 270

| | | |
|---|---|---|
| Grade 0 | 90 | (55.2) |
| Grade 1B | 1 | (0.6) |

Day 330

| | | |
|---|---|---|
| Grade 0 | 77 | (47.2) |
| Grade 1A | 2 | (1.2) |
| Grade 1B | 1 | (0.6) |

Day 365

| | | |
|---|---|---|
| Grade 0 | 80 | (49.1) |
| Grade 1A | 3 | (1.8) |

The few observations of mucosal edema, crusting, erythema, epistaxis, and nasal discharge were typically mild and similar to baseline. Odor identification results showed no clinically relevant olfactory changes in both age groups of interest (6-11 and ≥12 years), as shown in Table 8, where high scores signify better performance.

TABLE 8
NIH Odor Identification

| Time | | | | | |
|---|---|---|---|---|---|
| Point | Mean ± Standard Deviation (n) | | | | |
| (Day) | 5 mg | 10 mg | 15 mg | 20 mg | Total |

Ages 6 to 11

| 0 | 4.00 ± 0.0 (1) | 2.50 ± 2.12 (2) | 0 | | 5.00 ± 0.00 | (1) | 3.50 ± 1.73 | (4) |
|---|---|---|---|---|---|---|---|---|
| 30 | 2.00 ± 0.0 (1) | 3.50 ± 2.52 (4) | 9.00 ± 0.00 | (2) | 7.00 ± 0.00 | (1) | 5.13 ± 3.23 | (8) |
| 90 | 0 | 3.50 ± 1.29 (4) | 8.50 ± 0.71 | (2) | 2.00 ± 0.00 | (1) | 4.71 ± 2.81 | (7) |
| 150 | 0 | 3.00 ± 2.71 (4) | 7.50 ± 0.71 | (2) | 0 | | 4.50 ± 3.15 | (6) |
| 210 | 0 | 2.50 ± 2.38 (4) | 8.00 ± 0.00 | (2) | 1.00 ± 0.00 | (1) | 3.86 ± 3.34 | (7) |
| 270 | 0 | 3.00 ± 2.16 (4) | 5.50 ± 2.12 | (2) | 0 | | 3.83 ± 2.32 | (6) |
| 330 | 0 | 2.33 ± 1.53 (3) | 6.50 ± 0.71 | (2) | 6.00 ± 0.00 | (1) | 4.33 ± 2.42 | (6) |

| | | | | | | | |
|---|---|---|---|---|---|---|---|
| 365 | 0 | 2.00 ± 0.00 (2) | 8.00 ± 1.41 | (2) | 0 | 5.00 ± 3.56 | (4) |

Ages >12

| | | | | | | | | |
|---|---|---|---|---|---|---|---|---|
| 0 | 0 | 4.40 ± 2.19 (5) | 6.43 ± 2.29 | (21) | 7.09 ± 1.55 | (34) | 6.63 ± 2.00 | (60) |
| 30 | 0 | 6.00 ± 2.12 (5) | 6.36 ± 2.48 | (22) | 6.97 ± 1.89 | (35) | 6.68 ± 2.13 | (62) |
| 90 | 0 | 5.80 ± 2.49 (5) | 6.62 ± 2.04 | (21) | 7.14 ± 1.74 | (36) | 6.85 ± 1.92 | (62) |
| 150 | 0 | 5.80 ± 2.86 (5) | 6.33 ± 2.22 | (18) | 7.17 ± 1.70 | (30) | 6.75 ± 2.03 | (53) |
| 210 | 0 | 5.80 ± 2.05 (5) | 6.44 ± 2.34 | (16) | 6.79 ± 2.20 | (28) | 6.57 ± 2.21 | (49) |
| 270 | 0 | 5.40 ± 2.88 (5) | 6.05 ± 2.32 | (19) | 7.74 ± 1.48 | (27) | 6.88 ± 2.15 | (51) |
| 330 | 0 | 4.25 ± 1.71 (4) | 5.81 ± 2.74 | (16) | 7.00 ± 2.14 | (18) | 6.21 ± 2.48 | (38) |
| 365 | 0 | 5.33 ± 1.53 (3) | 5.94 ± 2.59 | (17) | 6.92 ± 2.17 | (24) | 6.43 ± 2.33 | (44) |

In these final data from a long-term study, diazepam nasal spray as rescue therapy for seizure clusters did not result in any new safety signals. AEs of specific relevance to clinicians and patients such as somnolence, nasal tolerability, and olfactory changes were infrequent and minimal in severity in patients aged 6-65 years. Additionally, no changes in respiratory rate, blood pressure, and heart rate were observed. These final data confirm those from previous analyses.

EXAMPLE 13: Lack of Impact of Seasonal Allergies or Rhinitis History on the Safety of Diazepam Nasal Spray in Patients with Seizure Clusters. As seasonal allergies are associated with nasal inflammation, it is important to establish that the safety of intranasal seizure rescue medications is not impacted by seasonal allergies. Diazepam nasal spray (Valtoco®) is formulated with Intravail® A3 (dodecyl maltoside, to enhance absorption) and vitamin E (to enhance solubility) to provide a rapid and noninvasive route of diazepam administration for acute treatment of seizure clusters in patients with epilepsy aged ≥6 years. Presented here are the final results of an exploratory analysis from the long-term, phase 3, open-label, repeat-dose safety study evaluating the impact of seasonal allergies or rhinitis history on the safety profile of diazepam nasal spray. The current investigation, with longer patient exposure across the seasons, updates a prior interim analysis.

Eligible patients were aged 6-65 years with a diagnosis of partial or generalized epilepsy with motor seizures or seizures with clear alteration of awareness who experienced seizure clusters. Patients and care partners were trained to administer age- and weight-based doses of 5, 10, 15, or 20 mg of diazepam nasal spray, with a second dose administered 4-12 hours later, if needed. TEAEs were recorded in a diary, and relationship to study drug was assessed. For this analysis, safety data were evaluated for subgroups of patients with or without seasonal allergies or rhinitis history, assessed overall and by season.

Of 175 patients enrolled in the study, 163 received ≥1 dose of diazepam nasal spray (54.6% female; mean age, 23.1 years) and were included in the safety population. Duration of exposure to diazepam nasal spray was ≥12 months (i.e., across all 4 seasons) in 81.6% of these patients. Within the safety population, 73 (44.8%) had seasonal allergies or rhinitis history. In this subgroup, 45 (61.6%) had TEAEs, 7 (9.6%) had serious TEAEs, and 1 (1.4%) had treatment-related TEAEs (nasal pruritus and rhinalgia) (Table 9).

TABLE 9
Most Common TEAEs in Patients
with or without Seasonal Allergies/Rhinitis

| | Seasonal Allergy | No Seasonal Allergy |
|---|---|---|
| | or Rhinitis | or Rhinitis |
| TEAEs, n (%) | (n = 73) | (n = 90) |
| Any TEAE | 45 (61.6) | 71 (78.9) |
| Serious TEAE | 7 (9.6) | 21 (23.3) |
| Death | 0 | 1 (1.1)* |
| Treatment-related TEAE | 1 (1.4) | 15 (16.7) |

Most common TEAEs (≥5% of patients in either group)

| | | |
|---|---|---|
| Upper respiratory tract infection | 15 (20.5) | 5 (5.6) |
| Seizure | 0 | 14 (15.6) |
| Pneumonia | 12 (16.4) | 0 |
| Nasopharyngitis | 9 (12.3) | 11 (12.2) |
| Influenza | 8 (11.0) | 5 (5.6) |
| Sinusitis | 7 (9.6) | 1 (1.1) |
| Dizziness | 0 | 7 (7.8) |
| Nasal discomfort | 0 | 7 (7.8) |
| Pyrexia | 0 | 6 (6.7) |
| Contusion | 0 | 5 (5.6) |
| Diarrhea | 0 | 5 (5.6) |

Treatment-related TEAEs (≥2 patients in either group)

| | | |
|---|---|---|
| Nasal discomfort | 0 | 7 (7.8) |
| Headache | 0 | 3 (3.3) |
| Epistaxis | 0 | 2 (2.2) |
| Migraine | 0 | 2 (2.2) |

*Not deemed treatment related.

In the subgroup of patients without allergies (n=90 [55.2%]), a total of 71 (78.9%) had TEAEs, 21 (23.3%) had serious TEAEs, and 15 (16.7%) had treatment-related TEAEs; in this subgroup, there was one death, which was not deemed treatment-related. There were no serious treatment-related TEAEs in either subgroup. TEAEs in the allergies subgroup occurred more frequently in the winter (from January-March) than in other seasons (Table 10).

TABLE 10
TEAEs by Season

| | |
|---|---|
| | Seasonal Allergy |
| | or Rhinitis |
| Season, n (%) | (n = 73) |

Overall TEAEs

| | |
|---|---|
| January-March (winter) | 27 (37.0) |
| April-June (spring) | 10 (13.7) |
| July-September (summer) | 13 (17.8) |
| October-December (fall) | 15 (20.5) |

Treatment-related TEAEs

| | |
|---|---|
| January-March (winter) | 0 |
| April-June (spring) | 1 (1.4) |
| July-September (summer) | 0 |
| October-December (fall) | 0 |

EXAMPLE 14: Timing to Administration and Ease of Dosing of Diazepam Nasal Spray Rescue Therapy for Seizure Clusters. Seizure clusters are emergencies associated with increased risk of prolonged seizures and status epilepticus, requiring prompt treatment to lower the risk of associated morbidities. Need for prompt treatment is reinforced by protocols recommending treatment in seizures lasting ≥5 minutes. Benzodiazepines are the cornerstone of treatment for seizure clusters. Diazepam nasal spray, formulated with Intravail® A3 (dodecyl maltoside) as an absorption enhancer and vitamin E for solubility, provides a quick, noninvasive, socially acceptable route of administration for acute treatment for seizure clusters in an easy-to-use portable device. Intranasal administration is quicker and avoids social challenges associated with rectal administration. This analysis of a long-term safety study examined the time to administration and ease of use of diazepam nasal spray for patients with epilepsy experiencing seizure clusters.

The study included patients aged 6-65 years with epilepsy and seizure clusters. Patients and caregivers were trained to administer diazepam nasal spray in age- and weight-based doses of 5, 10, 15, or 20 mg; second doses were to be administered 4-12 hours later if needed. A patient diary was used to record seizure timing, drug administration, and TEAEs. Surveys were distributed to patients and caregivers at study end, completion, or discontinuation to collect data on ease of use of diazepam nasal spray.

Of 175 patients enrolled, 163 received ≥1 dose of study treatment (54.6% female, mean age, 23.1 years; 27 [16.6%] reported self-administration). There were a total of 3853 seizure clusters treated with 4390 doses of diazepam nasal spray. Complete summary of time to administration statistics were available for 3627 seizure clusters lasting ≤24 hours (n=162 patients; range 1-143 seizure clusters per patient). Patients received treatment within 2 minutes of the start of 50% of seizure clusters, and 75% were treated within the first 5 minutes. For self-administering patients, 50% were treated within 2 minutes and 75% within 10 minutes. Eighty-four caregivers and 67 patients (including the 27 who self-administered treatment) responded to the exit survey (Table 11).

TABLE 11
Caregiver and Patient Survey Responses*

| | | |
|---|---|---|
| | | Self- |
| | | Administering |
| Characteristic | Caregivers† | Patients‡ |

Ease of administration of diazepam (%)

| | | |
|---|---|---|
| Extremely easy | 54.3 | 40.7 |
| Very easy | 39.5 | 37.0 |
| Somewhat easy | 4.9 | 14.8 |
| Slightly easy | 0 | 0 |
| Not at all easy | 1.2 | 7.4 |

*Surveys were provided to all patients enrolled in the study and their caregivers; caregivers and patients on the study at the time were given surveys at one time point toward the end of the study, to be returned at the next visit, and the surveys were mailed to those who had already completed or discontinued the study.
†n = 84 responded to the survey
‡n = 67 patients responded to the survey, and 27 reported self-administering diazepam nasal spray

Administration of diazepam nasal spray was rated extremely or very easy by 93.8% of caregivers and by 77.8% of patients that self-administered. Overall, treatment-related TEAEs were reported for 18.4% of patients (29.6% for patients who self-administered).

These results from the phase 3 safety study of diazepam nasal spray suggest that patients/caregivers administer diazepam rapidly (75% of seizures treated within 5 minutes). In the survey, ≥75% consider administration extremely or very easy and 40% of surveyed patients reported self-administration. This is meaningful for clinicians, as rapid time to seizure cluster control is critical to reduce morbidity and the risk of progression to status epilepticus and mortality.

EXAMPLE 15: Diazepam Nasal Spray (Valtoco®) Second Dose Administration Within 4 Hours: A Population Pharmacokinetic (PK) Analysis. Rectal diazepam has been an outpatient rescue therapy for seizure clusters for 2 decades but is limited by social considerations and PK variability. Diazepam nasal spray (Valtoco®) is now approved for acute treatment of seizure clusters in patients ≥6 years with epilepsy. Both product labels permit a second dose after 4 hours if needed. This schedule is based on safety and therapeutic estimates from the 1990s; more recent clinical diazepam nasal spray data included shorter dosing intervals, without safety signals. We developed a population PK model of diazepam nasal spray, identified sources of inter-individual variability (IIV), and performed simulation studies to estimate diazepam exposures after a second dose within shorter dosing intervals.

Diazepam PK data were analyzed from 3 phase 1 studies in healthy volunteers or patients with epilepsy. All participants received ≥1 dose of diazepam nasal spray (5, 10, 15, or 20 mg) based on age (6-11 years and ≥12 years) and weight. Population PK models were run using nonlinear mixed-effects modeling, with first-order conditional maximum likelihood estimation with interaction in NONMEM. In the covariate model, all relevant covariates with observed bias were tested separately, and all significant covariates were collectively added for the full covariate model. Parameter-covariate relationships were tested with backward selection. An exposure-response analysis was planned for AEs of interest after single and repeated doses in patients with epilepsy.

The final dataset included PK measurements from 126 individuals (Table 12), some receiving >1 dosage.

TABLE 12
Baseline Demographics, Dosing, and
Concomitant Therapies with Enzymatic Interaction

| | Patient Population | | |
|---|---|---|---|
| | Healthy | Patients with | |
| | Volunteers | Epilepsy | Total |
| n (male, female) | 78 (45, 33) | 48 (22, 26) | 126 (67, 59) |
| Age (years) | 36 (18-55) | 27.5 (6-59) | 33 (6-59) |
| Weight (kg) | 85 (52-109) | 67.6 (18.6-106) | 79 (18.6-109) |
| BMI (kg/m²) | 29.7 (19.5-44.3) | 24.4 (13.8-37.7) | 28.9 (13.8-44.3) |
| Dose strength- | 5 mg = 31 (Study 2) | 10 mg = 25 | 5 mg = 31 |
| Number of doses | 10 mg = 89 (Study 2) | 5 mg = 29 | 10 mg = 114 |
| | 15 mg = 17 (Study 3) | 20 mg = 41 | 15 mg = 46 |
| | 20 mg = 61 | | 20 mg = 102 |
| | (32 in Study 2; | | |
| | 29 in Study 3) | | |
| Concomitant CYP2C19 | NA | 24 | 24 |
| inhibitors* | | | |
| Concomitant | NA | 2 | 2 |
| CYP3A inhibitors* | | | |
| Concomitant | NA | 15 | 15 |
| CYP3A inducers* | | | |

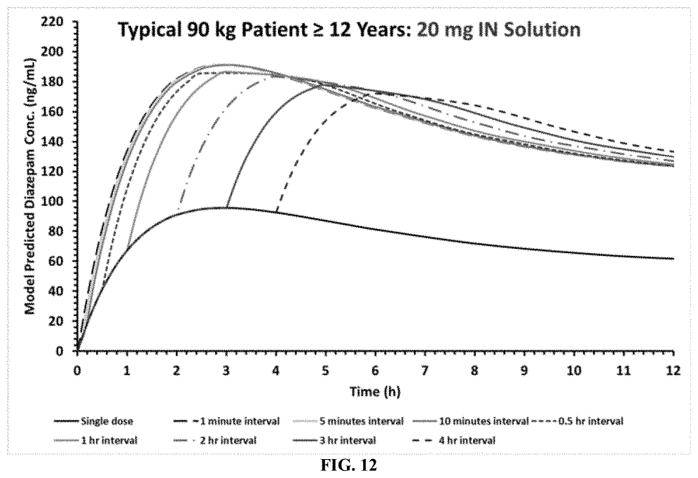

A two-compartment open PK model with first-order input and first-order elimination adequately fit the data. The model included clearance (CL), volume of distribution in central (V2) or peripheral compartments (V3), inter-compartmental clearance (Q), and first-order absorption rate constant (kₐ); weight was added as an allometric covariate. A final model that fit the observed PK was generated when population (volunteers or patients) was included as a covariate for kₐ. Point estimates of IIV were 41.8%, 45.5%, 47.1%, 66.2%, and 36.2% for CL, V2, V3, Q, and kₐ, respectively. As shown in FIG. 12, >90% of the observed data fell within the range of the 5^{th} and 95^{th} percentiles of the predicted data. In a simulation using the final PK model, a second dose of diazepam nasal spray increased maximum concentration (˜65%) and total exposure (˜100%). The predicted exposures from dosing intervals of 0.5-4 hour overlapped. There was no relationship between AEs and number of doses, and so the exposure-response analysis was not performed.

The PK of diazepam nasal spray is adequately described by a two-compartment model with first-order absorption and first-order elimination. This pattern was similar in healthy volunteers and patients with epilepsy. Dosing intervals of ≤4 hour were not associated with more safety events and are predicted to lead to comparable diazepam levels and exposures.

EXAMPLE 16: Development of a School Nurse Survey to Understand Current Practices, Barriers, and Needs for Treatment of Seizure Clusters in a School Setting. Children and adolescents with epilepsy may experience seizure clusters while at school. School nurses need to quickly implement personalized seizure action plans that include administering seizure rescue drugs. Intranasal benzodiazepine formulations are an effective alternative to rectal formulations, and may help better address social considerations and legal/policy restrictions in some school districts, especially when a registered nurse is not present. Diazepam nasal spray (Valtoco®) is approved for acute treatment of seizure clusters in patients with epilepsy aged ≥6 years. The purpose of our study was to evaluate the current practices among school nurses regarding their understanding and treatment of seizure clusters, and to identify the practice implications of using diazepam nasal spray to treat acute seizures in a school setting.

Drawing from the insights of a series of focus groups, a survey was developed to better understand the use of intranasal rescue medicine for seizures in the school health setting. The survey was emailed to approximately 40,000 school nurses across the US and included 33 questions related to frequency of in-school seizures, school requirements for seizure action plans, perceptions of ease and comfort of using diazepam nasal spray in a school setting (including student self-administration) and changes in school practice during off-campus activities, such as sports.

Results from the survey will identify barriers and solutions to in-school treatment of seizure clusters in order to promote educational initiatives for school nurses and other school personnel.

The data collected from this timely survey will provide information to guide the content of new training programs and the development of appropriate educational materials for school settings.

EXAMPLE 17: Safety of a Second Dose of Diazepam Nasal Spray within 4 Hours in Patients with Seizure Clusters. Second doses of diazepam rescue therapy for seizure clusters have historically been given 4-12 hours after the initial dose, if needed, based on the clinical development program for the rectal gel formulation approved by the US Food and Drug Administration in 1997. This timing was intended to address potential safety issues and drug blood levels. However, approximately one-third of second seizures in clusters take place within 3 hours of the first; thus, waiting to administer a second dose may be a limitation for the treating physician. Diazepam has been shown to be safe at a range of concentrations. In a long-term, phase 3, open-label, repeat-dose safety study of diazepam nasal spray, an exploratory analysis descriptively evaluated the safety profile of second doses within 4 hours of the first dose.

Enrolled patients were aged 6-65 years who experienced seizure clusters. Patients and care partners were trained to administer age- and weight-based doses of diazepam nasal spray of 5, 10, 15, or 20 mg; if needed, a second dose could have been administered 4-12 hours later. Per protocol, dosing could be adjusted as deemed appropriate by the investigator. This analysis assessed the safety of diazepam nasal spray in patients treated with ≥1 second dose within 4 hours and 4-24 hours after the first dose. ≥1 second dose within 4 hours and 4-24 hours after the first dose at any point during the study duration. TEAEs were recorded.

Of 175 enrolled patients, 163 received at least 1 dose (mean age, 23.1 years; 54.6% female). A total of 3853 seizure clusters were treated with a 4390 total doses of diazepam nasal spray. Of these doses, 485 were second doses given to 79 (48.5%) patients; 38 (23.3%) patients received ≥1 second dose within 4 hours (<4-hours group; 152 doses), and 41 (25.2%) patients received second doses at 4-24 hours (4-24-hours group; 333 doses) (all groups non-overlapping). Overall rates of TEAEs were generally similar between the groups (Table 13).

TABLE 13
TEAEs Reported for the Second Dose
in <4 Hours and 4-24 Hours Groups

| | <4 Hours | 4-24 Hours |
|---|---|---|
| Category, n (%) | (n = 38) | (n = 41) |
| Patients with TEAEs | 34 (89.5) | 33 (80.5) |
| Patients with serious TEAEs | 14 (36.8) | 14 (34.1) |
| Required/prolonged hospitalization | 13 (34.2) | 12 (29.3) |
| Treatment-related | 0 | 0 |
| Death | 0 | 1 (2.4)* |
| Discontinued due to TEAE | 0 | 1 (2.4)* |
| Patients with treatment-related TEAEs | 12 (31.6) | 7 (17.1) |

Most common treatment-related TEAEs (≥2 patients in either group)

| | | |
|---|---|---|
| Epistaxis | 3 (7.9) | 0 |
| Nasal discomfort | 3 (7.9) | 2 (4.9) |
| Headache | 2 (5.3) | 1 (2.4) |
| Rhinorrhea | 2 (5.3) | 0 |
| Somnolence | 2 (5.3) | 0 |
| Eye irritation | 0 | 2 (4.9) |
| Fatigue | 0 | 2 (4.9) |

The most common treatment-related TEAEs (≥2 patients) in the <4-hours group were of mild or moderate severity. Most common treatment-related TEAEs in the 4-24-hours group were rated as mild. One discontinuation due to a TEAE and one death (neither treatment-related) were reported in the 4-24-hours group; neither was reported in the <4-hours group. No serious treatment-related TEAEs were reported in either group.

These safety data from a long-term, phase 3, open-label, repeat-dose safety study found few treatment-related TEAEs in patients who received a second dose within 4 hours of the first dose or in patients who received second doses at 4-24 hours. These treatment-related TEAEs were typically mild or moderate. The results for both subgroups were consistent with an earlier interim analysis in the overall safety population and the established profile for rectal diazepam. These findings are reassuring for patients who received a second dose within 4 hours.

EXAMPLE 18: Assessment of Study Endpoints for Patients Discontinuing from a Phase 3, Long-Term, Open-Label, Repeat-Dose, Safety Study of Diazepam Nasal Spray for Acute Treatment of Seizure Clusters. Patients with severe and poorly controlled epilepsy are more likely to have seizure clusters, which are associated with increased risk for status epilepticus and death. This analysis evaluated patients from a phase 3, long-term, open-label, repeat-dose, safety study of diazepam nasal spray to compare results for those who completed the study with those who discontinued.

Eligible patients with epilepsy were aged 6-65 years and experienced seizure clusters. Patients and care partners were trained to administer age- and weight-based doses of 5, 10, 15, or 20 mg of diazepam nasal spray, with a second dose 4-12 hours later if needed. Proportion of seizures for which second doses were used was a proxy measure for effectiveness. Safety assessments included TEAEs and relationship to study drug. The safety population included patients who received ≥1 dose of diazepam nasal spray.

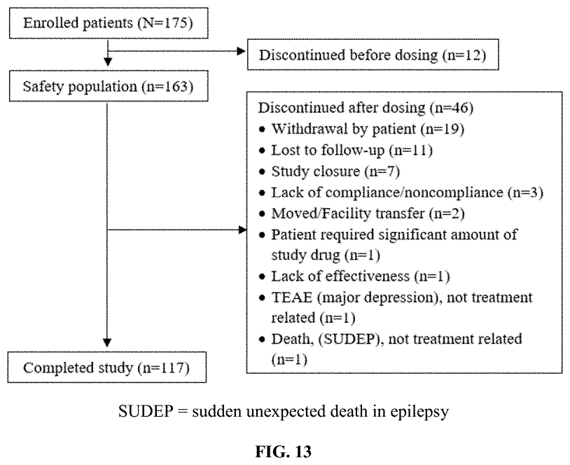

Of 175 patients enrolled, 163 were included in the safety population and had a total of 3853 seizure clusters, which were treated with a total of 4390 doses of diazepam nasal spray. Overall mean duration on the study was 17.4 months (range: 1.8-40.5 months). Of the 163 patients, 117 (71.8%) completed the study, and 46 (28.2%) discontinued prematurely. As shown in FIG. 13, the most common reasons for discontinuation (>15%) were withdrawal by patient (41.3% [19/46]), lost to follow-up (23.9% [11/46]), and study closure (15.2% [7/46]).

The mean age of the groups that completed or discontinued was similar at 23.6 years and 21.9 years, respectively. Duration of exposure was ≥12 months for 113 (96.6%) completers and 20 (43.5%) of those who discontinued. Mean doses per month was 2.3 for both groups. Proportion of second doses was similar at 12.6% (401 of 3195 seizure clusters) for completers and 12.8% (84 of 658 seizure clusters) for those who discontinued. TEAEs were reported for 104 (88.9%) completers, including 39 (33.3%) with serious TEAEs and 22 (18.8%) with treatment-related TEAEs (Table 14).

TABLE 14
Summary of TEAEs for Completers and
Those Who Discontinued (n = 163)

| | Completers | Discontinued |
|---|---|---|
| Category, n (%) | (n = 117) | (n = 46) |
| Total TEAEs | 104 (88.9) | 30 (65.2) |
| Serious TEAEs | 39 (33.3) | 11 (23.9) |
| Death | 0 | 1 (2.2)* |
| Discontinued due to TEAE | 0 | 1 (2.2)* |
| Treatment-related TEAEs | 22 (18.8) | 8 (17.4) |

Most common TEAEs (≥5% in either group)

| Seizure | 25 (21.4) | 6 (13.0) |
|---|---|---|
| Upper respiratory tract infection | 18 (15.4) | 2 (4.3) |
| Nasopharyngitis | 16 (13.7) | 4 (8.7) |
| Pyrexia | 14 (12.0) | 3 (6.5) |
| Influenza | 10 (8.5) | 3 (6.5) |
| Pneumonia | 10 (8.5) | 2 (4.3) |
| Urinary tract infection | 9 (7.7) | 2 (4.3) |
| Somnolence | 10 (8.5) | 1 (2.2) |
| Sinusitis | 7 (6.0) | 1 (2.2) |
| Status epilepticus | 7 (6.0) | 0 |
| Vomiting | 7 (6.0) | 2 (4.3) |
| Constipation | 6 (5.1) | 2 (4.3) |
| Contusion | 6 (5.1) | 0 |
| Diarrhea | 6 (5.1) | 3 (6.5) |
| Ear infection | 6 (5.1) | 1 (2.2) |
| Epilepsy | 6 (5.1) | 0 |
| Fall | 6 (5.1) | 0 |
| Nausea | 6 (5.1) | 2 (4.3) |
| Dizziness | 5 (4.3) | 6 (13.0) |
| Nasal discomfort | 5 (4.3) | 5 (10.9) |
| Cough | 5 (4.3) | 3 (6.5) |
| Most common treatment-related TEAEs | | |
| (>2 patients in either group) | | |
| Nasal discomfort | 5 (4.3) | 5 (10.9) |
| Dysgeusia | 3 (2.6) | 0 |
| Somnolence | 3 (2.6) | 0 |

*Not deemed treatment related.

Among those who discontinued, the proportions of TEAEs were consistently lower: TEAEs were reported for 30 (65.2%), including 11 (23.9%) with serious TEAEs and 8 (17.4%) with treatment-related TEAEs. Among the discontinuations, one was due to an AE of major depression and one to sudden unexpected death in epilepsy; neither of these was treatment related.

These results from a long-term safety study demonstrate similarities in safety and effectiveness of diazepam nasal spray between completers and those who discontinued. Only one discontinuation was due to a TEAE and one to death, neither of which were deemed treatment related. These results suggest that discontinuations had minimal effect on either the safety or effectiveness of diazepam nasal spray.

EXAMPLE 19: Evaluation of Diazepam Nasal Spray in Patients with Seizure Clusters Concomitantly Receiving Clobazam. Benzodiazepines are a mainstay of seizure-cluster treatment and are also included in some daily anti-seizure drug (ASD) regimens. Thus, it is clinically relevant to understand whether diazepam nasal spray may be affected by other benzodiazepines, including chronic treatment with clobazam. This sub-analysis from a long-term safety study evaluates the effectiveness and safety of diazepam nasal spray in subgroups of patients receiving clobazam or other benzodiazepines.

Data from a phase 3, open-label, repeat-dose safety study of diazepam nasal spray were analyzed by subgroups receiving chronic clobazam or other intermittent and chronic benzodiazepines. Patients were aged 6-65 years with frequent seizure clusters despite stable ASDs. Care partners and patients were trained to administer diazepam nasal spray (5, 10, 15, or 20 mg), with second doses 4-12 hours later if needed. Investigators could adjust doses for effectiveness or safety reasons. Seizures, drug administration, and TEAEs were recorded in seizure diaries. Second doses were assessed during the 24 hours after the seizure.

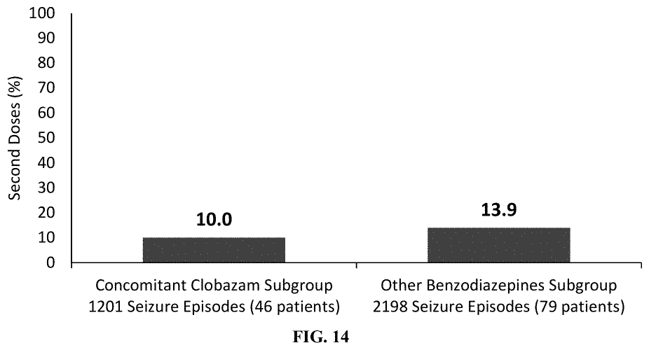

Among 175 enrolled patients, 163 were treated. Of these, 125 patients (52.8% female) are included in the current analysis; 46 (36.8%; mean age, 17.7 years) used clobazam and 79 (63.2%; mean age, 26.0 years) used other benzodiazepines. Exposure to diazepam nasal spray was ≥12 months in most patients (clobazam, 81.0%; other benzodiazepines, 91.3%) and retention rates (study completion irrespective of study closure) were 72.2% and 80.4% in the clobazam and other benzodiazepine subgroups, respectively. As shown in FIG. 14, The subgroups had similar mean total doses per patient (clobazam, 26.1; other benzodiazepines, 27.8) and doses per month (clobazam, 2.5; other benzodiazepines, 2.3). In both subgroups, <15% of seizure episodes were treated with a second dose.

The proportion of patients with TEAEs was slightly higher in the clobazam subgroup (89.1% vs 83.5%); however, treatment-related TEAE rates were similar (˜20%) (Table 15). Serious TEAEs were higher in the clobazam subgroup (47.8% vs 25.3%), but no serious TEAEs were deemed treatment-related. No events of respiratory depression were reported. The only treatment-related TEAT in ≥5% of patients in either group was nasal discomfort (8.9%, other benzodiazepines; 4.3%, clobazam).

TABLE 15
TEAEs in Patients Treated with Diazepam Nasal Spray.

| | Number (%) of Patients | |
|---|---|---|
| | Concomitant | Other Concomitant |
| | Clobazam | Benzodiazepines |
| TEAE | Subgroup (n = 46) | Subgroup (n = 79) |
| Any TEAE | 41 (89.1) | 66 (83.5) |
| Serious TEAE | 22 (47.8) | 20 (25.3) |
| Treatment-related TEAE | 9 (19.6) | 17 (21.5) |

Most common TEAEs (≥10% in either subgroup)

| Seizure | 13 (28.3) | 13 (16.5) |
|---|---|---|
| Nasopharyngitis | 11 (23.9) | 3 (3.8) |
| Upper respiratory tract | 10 (21.7) | 7 (8.9) |
| infection | | |
| Nasal discomfort | 2 (4.3) | 7 (8.9) |
| Pneumonia | 7 (15.2) | 4 (5.1) |
| Pyrexia | 7 (15.2) | 8 (10.1) |
| Urinary tract infection | 7 (15.2) | 3 (3.8) |
| Influenza | 6 (13.0) | 6 (7.6) |
| Most common treatment-related | | |
| TEAEs ≥2 patients in either subgroup) | | |
| Nasal discomfort | 2 (4.3) | 7 (8.9) |
| Dysgeusia | 0 | 3 (3.8) |
| Headache | 0 | 3 (3.8) |
| Migraine | 0 | 2 (2.5) |
| Rhinalgia | 0 | 2 (2.5) |
| Somnolence | 0 | 2 (2.5) |

Final results from this long-term study show that the effectiveness and safety profile of diazepam nasal spray was not substantially altered by concomitant chronic clobazam treatment. There was no notable effect of chronic clobazam on the number of second doses needed per episode (used as a proxy of effectiveness), on the safety/tolerability profile of diazepam nasal spray, or study retention compared with other chronic or intermittent benzodiazepines or with the overall study.

EXAMPLE 20: QoL and Treatment Satisfaction in a Long-Term Safety Study of Diazepam Nasal Spray for Seizure Clusters as Assessed by Patients and Their Caregivers. Patients with seizure clusters may experience reduced quality of life (QoL) and could benefit from a rescue treatment that can be easily administered in any setting. A long-term safety study of diazepam nasal spray examined Quality of Life in Epilepsy (QOLIE) and treatment satisfaction among patients and caregivers.

Patients (6-65 years) in a long-term, open-label safety study of diazepam nasal spray used 5-, 10-, 15- or 20-mg doses (based on age and weight) to treat seizure clusters. QOLIE-31-P and QOLIE-48 are epilepsy-specific instruments to assess health-related QoL in adults (≥18 years) and adolescents (11-17 years), respectively. Total scores, on a 100-point scale, are a weighted composite of 7 (QOLIE-31-P) or 8 (QOLIE-48) 100-point subscales. Higher scores indicate better QoL. Patients completed the age-appropriate assessment at baseline (Day 0) and Days 30, 150, 270 and 365. In addition, near study end or after study completion/discontinuation, patient and caregiver pairs completed surveys assessing use of rescue medications and comfort with/ease of using diazepam nasal spray that affect QoL. Descriptive statistics were calculated on QOLIE and survey data for the subset of patients with QOLIE baseline data (Day 0 or 30), survey data, and a caregiver-completed survey.

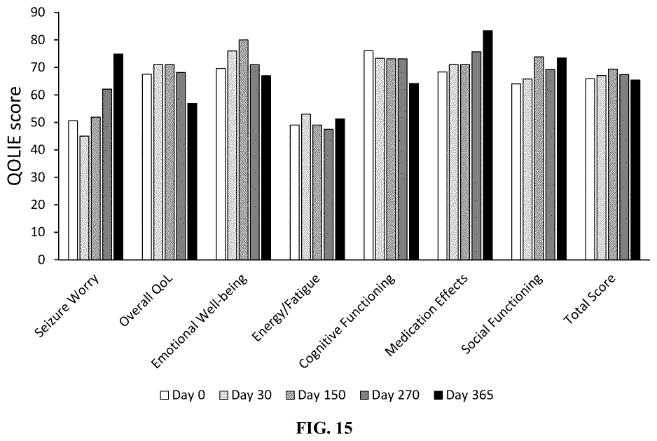

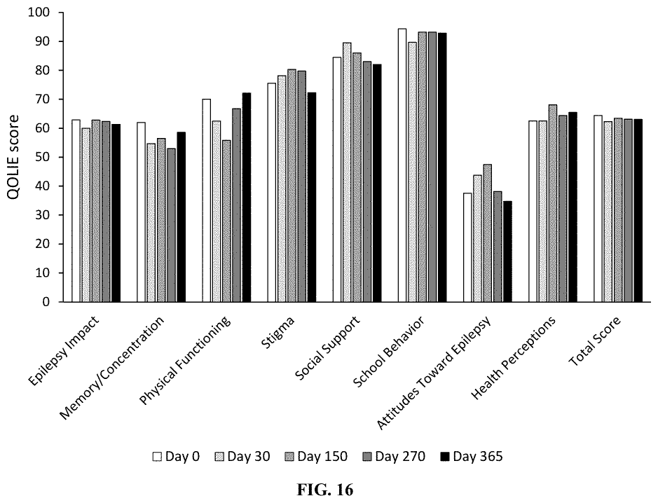

Of 163 treated patients, 5 adults and 14 adolescents and their caregivers provided data for the QOLIE and the survey analyses. As shown in FIG. 15, Mean total QOLIE-31-P score was stable across time with a numerical trend toward improvement from baseline in mean Seizure Worry sub score. Mean total QOLIE-48 score also remained stable across time with no consistent trends in subscale scores, as shown in FIG. 16. Survey responses on comfort carrying diazepam nasal spray outside the home showed most patients and caregivers were extremely or very comfortable (84.2% and 88.9%, respectively); responses on comfort doing activities outside the home while having diazepam nasal spray available showed most patients and caregivers were extremely or very comfortable (94.7% and 88.9%, respectively).

In the study overall, tolerability of diazepam nasal spray was similar to rectal diazepam; no treatment-related AEs were considered serious or led to discontinuation.

In this small subset analysis from a long-term safety study, QOLIE scores generally remained stable with a trend toward improvement in Seizure Worry in the adults. Based on survey data, patients and caregivers had a high degree of comfort carrying diazepam nasal spray and doing daily activities outside the home, benefiting QoL. QOLIE assessments may be less sensitive for measuring change with an intermittent rescue therapy compared with a survey geared towards assessing comfort with treatment. Both types of measures suggest diazepam nasal spray availability may decrease concern about seizure occurrence.

EXAMPLE 21: Lack of Tolerance with Diazepam Nasal Spray for Seizure Clusters After Long-term Use. Long-term effectiveness of benzodiazepine rescue therapy for seizure clusters may be affected if tolerance develops. Diazepam nasal spray (Valtoco®) is approved for acute treatment of seizure clusters in patients aged ≥6 years with epilepsy and is designed to provide a rapid, noninvasive, and socially acceptable route of administration. This large, final analysis from a long-term phase 3 safety study of diazepam nasal spray updates prior results to assess whether use of a second dose, as a proxy for effectiveness, is maintained.

Patients aged 6-65 years with epilepsy and seizure clusters were enrolled. Patients and caregivers were trained to administer age- and weight-based doses of diazepam nasal spray; if needed, a second dose could be given 4-12 hours later. Tolerance was assessed in 2 adjacent periods (period 1 [initial] and period 2 [subsequent]) for each patient and comparing the proportion of events for which second doses were used in periods 1 and 2. Two methods were used to define “initial” and “subsequent” in both periods: (1) minimum number of events and (2) specific number of months. For all methods, consideration was restricted to subjects with ≥8 events in the initial period. Seizure clusters were defined to include any seizures within 24 hours of the initial event.

Of 175 patients enrolled, 163 were treated with diazepam nasal spray for 3853 seizure-clusters during a mean of 1.5 years. Based on the range of exposure across the patient population, 256 analyses were conducted with time cutoffs from 4-36 months in each period (totals of 8-72 months) and 1-22 events in each period (totals of 2-44 events; Table 16). Only 6 (2.3%) analyses showed nominally significant changes (P<0.05) in number of second doses between periods 1 and 2; fewer than expected by chance. Of nominally significant changes, period 2 mean rate was greater than period 1 in 4 instances and smaller in 2 instances. Across all analyses, rate of second doses was generally lower in period 2, with fewer second doses in period 2 for 167 (65.2%) analyses and fewer second doses in period 1 for 89 (34.8%) analyses. TEAEs occurred in 134 (82.2%) patients. One patient discontinued due to a TEAE (major depression), deemed not treatment related. One death occurred (sudden unexpected death in epilepsy), deemed unlikely related to treatment. None of the treatment-related TEAEs (30 [18.4%] patients) were serious.

TABLE 16
Summary^{a} of Mean Percentage of Events Treated with Second Doses, Averaged Over Patients,
based on the Cutoff with the Highest Number of Events Analyzed in Each Time Period

| | | | | | | | | Wilcoxon |
|---|---|---|---|---|---|---|---|---|
| | Number | | Period 1 | | Period 2 | | | Signed |
| Month | of Events | Patients, | Mean, | Max | Mean, | Max | Difference, | Rank Test |
| Cutoff | Cutoff | n^{b} | n (%) | Events, n | n (%) | Events, n | % (P2 − P1) | P value |
| 4 | 7 | 12 | 9.8 (10.1) | 14 | 10.3 (18.2) | 17 | 8.1 | 0.195 |
| 6 | 10 | 8 | 14.1 (10.5) | 18 | 13.6 (22.5) | 17 | 12.0 | 0.063 |
| 8 | 13 | 9 | 19.0 (25.3) | 28 | 18.4 (28.3) | 25 | 3.1 | 0.844 |
| 10 | 18 | 9 | 24.6 (27.5) | 32 | 24.0 (23.7) | 38 | −3.8 | 0.129 |
| 12 | 19 | 8 | 28.0 (27.9) | 32 | 28.3 (21.7) | 47 | −6.2 | 0.039 |
| 14 | 18 | 8 | 27.6 (20.9) | 36 | 29.3 (18.1) | 53 | −2.8 | 0.547 |
| 16 | 19 | 8 | 32.5 (19.2) | 45 | 31.6 (14.2) | 56 | −4.9 | 0.469 |
| 18 | 18 | 8 | 32.5 (14.2) | 52 | 34.4 (9.5) | 64 | −4.7 | 0.156 |
| 20 | 18 | 9 | 34.7 (16.7) | 61 | 37.4 (15.2) | 74 | −1.5 | 0.813 |
| 22 | 20 | 8 | 39.1 (16.2) | 69 | 40.4 (14.2) | 73 | −2.0 | 0.563 |
| 24 | 22 | 8 | 40.6 (16.7) | 74 | 42.9 (13.3) | 77 | −3.4 | 0.438 |
| 26 | 14 | 8 | 41.8 (16.5) | 81 | 37.9 (18.9) | 82 | 2.4 | 0.844 |
| 28 | 15 | 8 | 43.0 (16.6) | 82 | 45.9 (17.7) | 99 | 1.0 | 0.844 |
| 30 | 11 | 8 | 43.6 (16.4) | 86 | 45.8 (18.4) | 102 | 1.9 | 0.938 |
| 32 | 13 | 8 | 47.9 (16.1) | 94 | 46.1 (19.1) | 107 | 3.0 | 0.938 |
| 34 | 15 | 8 | 50.6 (16.5) | 100 | 47.8 (19.2) | 108 | 2.7 | 0.813 |
| 36 | 6 | 8 | 55.0 (14.4) | 109 | 43.3 (18.9) | 112 | 4.5 | 0.813 |

^{a}Summary of all 256 analyses
^{b}Minimum 8 subjects

This array of analyses of final data from a phase 3 safety study on use of a second dose of diazepam nasal spray suggests that effectiveness is retained over time for treatment of seizure clusters, with no evidence of tolerance. No safety signals emerged with continued use. Across multiple analyses, only 2.3% were statistically nominally significant, below the 5% level suggested by chance. Moreover, observed changes were directionally inconsistent, suggesting that the nominally significant differences observed were random.

EXAMPLE 22: Quality of Life in Epilepsy Scale for Frequent and Infrequent Users of Diazepam Nasal Spray for Seizure Clusters. Rationale: Despite appropriate anti-seizure drugs, seizure clusters can disrupt daily living and health-related quality of life (QoL). This analysis of a long-term safety study assessed scores on the Quality of Life in Epilepsy scale (QOLIE) in adults with epilepsy in subgroups based on frequency of use of diazepam nasal spray to treat seizure clusters.

A long-term, open-label, repeat-dose safety study of diazepam nasal spray enrolled patients with epilepsy aged 6-65 years. Participants received age- and weight-based doses of diazepam nasal spray for outpatient treatment of seizure clusters. Frequent treatment was defined as an average of ≥2 doses/month. The QOLIE-31-P, a self-reported tool for adults (≥18 years), uses a 4-week recall period, and was administered on Days 0 (baseline), 30, 150, 270, and 365. The overall score (100-point scale; higher scores signify better QoL) and 7 subscale scores (Seizure Worry, Overall QoL, Emotional Well-Being, Energy/Fatigue, Cognitive Functioning, Medication Effects, Social Functioning) were analyzed in frequent and infrequent users of diazepam nasal spray; in particular, Seizure Worry and Social Functioning were examined because they might be expected to be the most relevant to control of intermittent seizure clusters. Descriptive statistics were calculated, and the 2-sided, 2-sample t test was used for comparison of frequent and infrequent users of diazepam nasal spray.

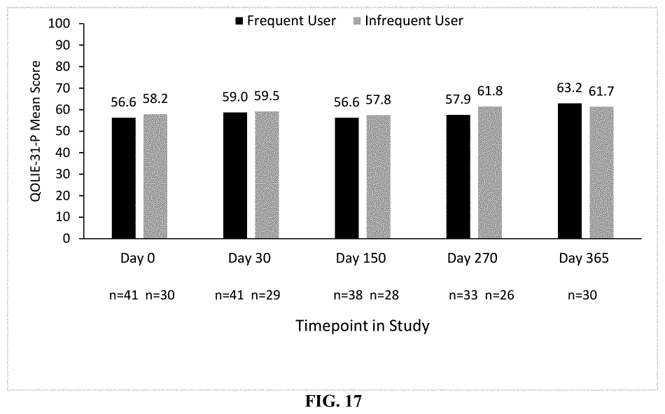

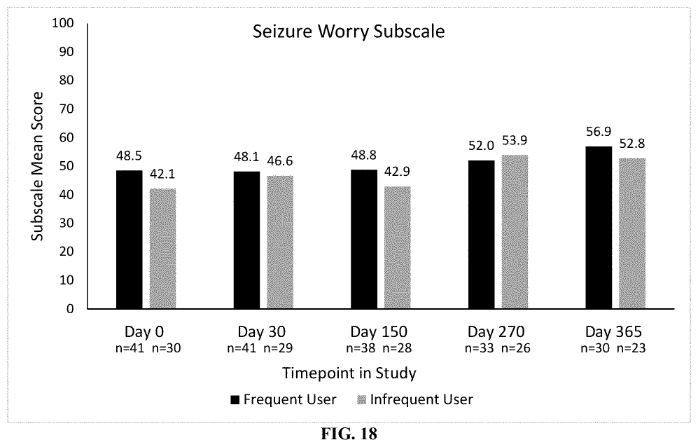

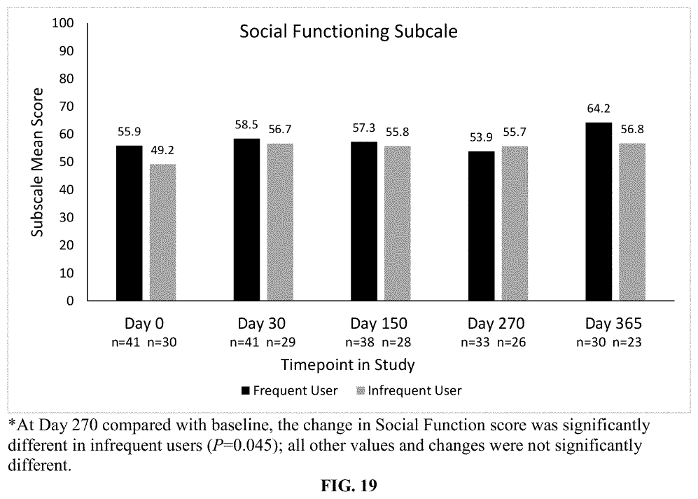

Of 175 enrolled patients, 163 pediatric (n=78) and adult (n=85) patients were treated; 117 completed the study. The QOLIE-31-P was completed by 74 adults (median age, 33 y [range, 18-65]; female, 59.5%) at one or more time points. There were 41 frequent and 33 infrequent users of diazepam nasal spray among QOLIE-31-P respondents (mean [SD] doses per month were 3.2 [1.80] and 1.3 [0.33], respectively). Overall QOLIE-31-P scores were similar between frequent and infrequent users and were slightly higher (improved) in both groups during the study, as shown in FIG. 17. Mean values for Seizure Worry and Social Function scores for QOLIE-31-P were similar between frequent and infrequent users and rose slightly from baseline, as shown in FIG. 18 and FIG. 19. Tolerability was similar to the full study, with 23.0% reporting treatment-related TEAEs; none were serious or led to discontinuation.

In both frequent and infrequent groups, QOLIE-31-P scores were maintained, improving slightly during a year with diazepam nasal spray available as a rescue treatment for seizure clusters, suggesting a favorable relationship between benefit and tolerability that is relevant for patients with epilepsy. Patients with less frequent seizure clusters (about 1.3 per 28-day recall period) had higher baseline overall QOLIE scores; however, overall improvement was higher in the frequent usage group.

EXAMPLE 23: Safety and Effectiveness of Diazepam Nasal Spray in Pediatric Patients with Epileptic Encephalopathy. Approximately one third of pediatric patients with epilepsy develop refractory epilepsy that can be associated with medically resistant seizures (with or without clustering) and impaired development. This analysis assessed the safety and effectiveness of diazepam nasal spray in pediatric patients with epileptic encephalopathy from a long-term, phase 3, open-label, repeat-dose, safety study.

The study enrolled patients aged 6-65 years with frequent seizure clusters. Care partners and patients were trained to administer doses of 5, 10, 15, or 20 mg of diazepam nasal spray based on age (≤11 or ≥12 years) and weight, with instructions to administer second doses 4-12 hours later if needed. Investigators could adjust doses for effectiveness or safety reasons. Seizures, drug administration, and TEAEs were recorded in seizure diaries. Use of a second dose within 24 hours for a seizure cluster was a proxy measure for effectiveness. The epileptic encephalopathy subgroup was determined based on the patients' medical histories.

The study enrolled 175 adult and pediatric patients, with a safety population of 163 treated patients that included 78 patients aged 6-17 years. A total of 64 (39.3%) of these patients were included in the pediatric epileptic encephalopathy subgroup (54.7% female; mean age [SD], 10.1 [3.2]; age range 6-17 years; Table 17).

TABLE 17
Demographics and Duration of Exposure
for Pediatric Patients with Encephalopathies

| | | Pediatric Patients with |
|---|---|---|
| | | Encephalopathies |
| Characteristics | | (n = 64) |
| Sex | | |
| Male | | 29 (45.3) |
| Female | | 35 (54.7) |
| Age, years | | |
| Mean (SD) | | 10.1 (3.2) |
| Median | | 10.0 |
| Range | | 6-17 |
| Weight, kg^{a} | | |
| Mean (SD) | | 32.6 (15.7) |
| Median | | 27.5 |
| Range | | 12-78 |
| Duration of exposure, n (%) | | |
| <6 | months | 3 (4.7) |
| 6 to <12 | months | 9 (14.1) |
| ≥12 | months | 52 (81.3) |

^{a}n = 63

Duration of exposure to diazepam nasal spray in this subgroup was ≥12 months in 52 (81.3%) patients. Mean (SD) number of doses of diazepam nasal spray per month were 2.3 (1.4). There were 1402 treated seizure clusters in this subgroup; 149 (10.6%) were treated with a second dose.

TEAEs were reported in 57 (89.1%) patients in this pediatric epileptic encephalopathy subgroup, including 25 (39.1%) with serious TEAEs and 10 (15.6%) with treatment-related TEAEs (Table 18).

TABLE 18
TEAEs in Pediatric Patients with Encephalopathies

| | Pediatric Patients |
|---|---|
| | with |
| TEAEs, | Encephalopathies |
| n (%) | (n = 64) |
| ≥1 TEAE | 57 (89.1) |
| TEAE leading to discontinuation | 0 |
| Serious TEAE | 25 (39.1) |
| Treatment related | 0 |
| Death | 0 |

Most common TEAEs (≥5%)

| | |
|---|---|
| Seizure | 17 (26.6) |
| Nasopharyngitis | 14 (21.9) |
| Pyrexia | 13 (20.3) |
| Upper respiratory tract infection | 10 (15.6) |
| Influenza | 9 (14.1) |
| Pneumonia | 8 (12.5) |
| Constipation | 7 (10.9) |
| Vomiting | 7 (10.9) |
| Diarrhea | 6 (9.4) |
| Pharyngitis streptococcal | 6 (9.4) |
| Somnolence | 6 (9.4) |
| Urinary tract infection | 6 (9.4) |
| Ear infection | 5 (7.8) |
| Sinusitis | 4 (6.3) |
| Cough | 5 (7.8) |
| Status epilepticus | 5 (7.8) |
| Treatment-related TEAEs | 10 (15.6) |
| Epistaxis | 2 (3.1) |
| Dysgeusia | 1 (1.6) |
| Eye irritation | 1 (1.6) |
| Nasal discomfort | 1 (1.6) |
| Nasal mucosal disorder | 1 (1.6) |
| Rash | 1 (1.6) |
| Rhinorrhea | 1 (1.6) |
| Sensory integrative dysfunction | 1 (1.6) |
| Somnolence | 1 (1.6) |
| Tonsillar hypertrophy | 1 (1.6) |

The most common TEAEs (≥10 patients) were seizure, 17 (26.6%); nasopharyngitis, 14 (21.9%); pyrexia, 13 (20.3%); and upper respiratory tract infection, 10 (15.6%). The only treatment-related TEAE in ≥1 patient was epistaxis (n=2). In this sub-analysis, there were no treatment-related serious TEAEs and no discontinuations due to TEAEs or deaths. Forty-three patients completed the study, and 7 discontinued due to study closure, for a retention rate in this subgroup up to study closure of 78.1%.

In an analysis of the subgroup of pediatric patients with epileptic encephalopathy from the long-term, phase 3, safety study of diazepam nasal spray, the high percentage of seizure clusters not using a second dose suggests initial-dose effectiveness in this highly intractable subgroup. There were no safety signals compared with previous diazepam formulations for treatment of seizure clusters, with a high retention rate of 78.1% in this long-term study.

EXAMPLE 24: Single-Dose Crossover Study to Evaluate Relative Biocompatability of Two Dose Actuation Formulations Versus Single Dose Actuation Formulations of Diazepam Nasal Spray.

Background

Epilepsy is a significant health problem affecting 50 million people worldwide, including 2.7 million Americans. Epilepsy negatively impacts quality of life and increases morbidity and mortality. In the US, 25,000 to 50,000 deaths each year are attributed to seizures and related causes. Seizure emergencies include status epilepticus, prolonged seizures and acute repetitive seizures ([ARS], also known as cluster seizures). The IV formulation of diazepam has been used for over 30 years in the treatment of seizure emergencies around the world, including status epilepticus, but the current standard of care formulations for the outpatient management of cluster seizures includes the use in the US of a rectal gel formulation of diazepam, Diastat® (Diastat-2016) approved in 1997, diazepam nasal spray, Valtoco® (Valtoco-2021) approved in 2020, and a midazolam nasal spray, Nayzilam® (Nayzilam-2021) approved in 2019.

Valtoco is indicated for the acute treatment of intermittent, stereotypic episodes of frequent seizure activity (i.e., seizure clusters, acute repetitive seizures) that are distinct from a patient's usual seizure pattern in patients with epilepsy 6 years of age and older. Diazepam, the active ingredient of Valtoco nasal spray, is a benzodiazepine anticonvulsant with the chemical name 7-chloro-1,3-dihydro-1-methyl-5-phenyl-2H-1,4-benzodiazepin-2-one. Intravail A3® is included in the Valtoco formulations as an absorption enhancement agent for intranasal delivery of diazepam (Investigator's Brochure 2020). Intravail A3 (dodecylmaltoside) belongs to a class of nonionic surfactants known as alkylglycosides. Alkylglycosides, which consist of alkyl chains of various lengths linked to a sugar moiety, have been extensively studied for their ability to promote increased bioavailability of drugs via the nasal, oral, and ocular routes. Dodecylmaltoside is the glycoside of maltose and the long chain alcohol dodecanol. The chemical name of Intravail is 1-O-n-dodecyl-β-D-Maltopyranoside (CAS #69227-93-6). Synonyms are Dodecyl-4-O-α-D-glucopyranosyl-β-D-glucopyranoside, dodecylmaltoside, and n-Dodecyl β-D-maltoside.

Previous pharmacokinetic information for Valtoco following nasal administration was obtained from studies conducted in healthy adult subjects, as well as adult and pediatric patients with epilepsy 6 years of age and older. In a PK study in healthy adult subjects, the highest plasma diazepam concentrations after nasal administration of Valtoco were reached in 1.5 hours. The estimated volume of distribution of diazepam at steady-state is 0.8 to 1.0 μL/kg. The absolute bioavailability of Valtoco relative to intravenous diazepam was 97%. The t½ of diazepam following administration of a 10 mg dose of Valtoco was found to be about 49.2 hours. In another PK study in healthy adult subjects, diazepam plasma exposures (Cmax and AUC) increased approximately proportional to dose from 5 mg to 20 mg. In a relative bioavailability study in healthy adult subjects, diazepam exposure (Cmax and AUCs) was evaluated following administration of 15 and 20 mg of Valtoco nasal spray and diazepam rectal gel. The diazepam PK parameters were 2- to 4-fold less variable for Valtoco and within the range of those seen with diazepam rectal gel. In a pharmacokinetic study in patients with epilepsy, PK parameters were similar between seizure versus non-seizure states.

Rationale and Objectives

A diazepam nasal spray has been developed for patients who experience ARS to provide a convenient and acceptable route of diazepam administration compared to alternative routes of administration, such as rectal gel. Doses of Valtoco 20 mg are currently administered using a series of 2 intranasal spray applications of 10 mg into each nostril (Reference). The current study is planned to support the development of a single intranasal spray administration of 20-mg dose of Valtoco (Test) using 3 different amounts of an absorption enhancer and to determine the relative bioavailability of the Test and Reference formulations. The formulations are listed in TABLE 19. The Reference formulation (Formulation A, TABLE 19) will be replicated in each sequence to obtain the within-subject variability deviation for the Reference formulation.

TABLE 19

| | Solution Identifier | | | |
|---|---|---|---|---|
| Component | A | B | C | D |
| Number of doses | 2 | 1 | 1 | 1 |
| diazepam (% w/v) | 10 | 20 | 20 | 20 |
| in each dose | | | | |
| alkyl maltoside (% w/v) | 0.25 | 0.25 | 0.50 | 0.75 |
| in each dose | | | | |
| vitamin E (% w/v) | 56.47 | 41.86 | 41.68 | 41.50 |
| in each dose | | | | |
| ethanol (% w/v) | q.s. 100 μL | 0 | 0 | 0 |
| in each dose | | | | |
| benzyl alcohol (% w/v) | 10.5 | 41.50 | 41.50 | 41.50 |
| in each dose | | | | |

The primary objectives of this study are to characterize differences in the PK of diazepam and evaluate relative biocomparability after administration of 20 mg of diazepam using the test and reference formulations of Valtoco nasal spray in healthy subjects. The secondary objectives are to 1) determine whether two 10-mg actuations of the current Valtoco formulation (Reference Formulation A) are bioequivalent to a single 20-mg actuation of a new Valtoco formulation (Test Formulations B, C, D, Table 19) using different amounts of absorption enhancer, administered nasally in healthy subjects; and 2) evaluate the safety and tolerability of a single 20-mg actuation of Valtoco using different amounts of absorption enhancer, administered nasally in healthy subjects.

Study Design

This is a pilot, Phase 1, open-label, randomized, 5-period, partial replicate crossover study in healthy subjects to determine the relative biocomparability of 2 nasal spray actuations of the current formulation of Valtoco (Reference Formulation A) and 1 nasal spray actuation of 3 new formulations of Valtoco (Test Formulations B, C, and D). The bioequivalence in terms of diazepam, as well as the safety and tolerability of 20 mg of diazepam, using the Test and Reference formulations of Valtoco nasal spray was evaluated.

Approximately 25 eligible subjects, with a body weight of 76 to 111 kg (inclusive) and BMI of 18 to 32, were randomized after an overnight fast of at least 8 hours into 1 of 10 treatment sequences (Table 20) and received a single dose of Valtoco, either via 2 intranasal spray applications (1 device each) or 1 intranasal spray application in the 5 treatment periods (Treatment Periods 1, 2, 3, 4, and 5). Subjects were dosed on Day 1 of each of the Periods 1 through 5. Treatments were separated by a minimum 14-day washout period.

TABLE 20

| Treatment | No. of | | | | | |
|---|---|---|---|---|---|---|
| Sequence | Subjects | Period 1 | Period 2 | Period 3 | Period 4 | Period 5 |
| 1 | 3 | A | B | A | C | D |
| 2 | 3 | A | D | A | C | B |
| 3 | 3 | C | D | B | A | A |
| 4 | 3 | B | A | C | A | D |
| 5 | 3 | A | A | D | B | C |
| 6 | 3 | D | C | A | B | A |
| 7 | 3 | B | C | A | D | A |
| 8 | 3 | A | A | B | D | C |
| 9 | 3 | D | A | C | A | B |
| 10 | 3 | C | B | D | A | A |

Safety assessments were performed at each of the study visits. Subjects were followed until Day 14 of Treatment Period 5. Nasal assessments were conducted after each administration of study drug. The scoring was done by a trained observer based on an assessment of the nasal mucosa. Irritation was assessed using a 6-point scoring system by evaluating the degree of mucosal irritation, inflammation, erosion, ulceration, and septal perforation. Nasal mucosal epistaxis, edema, discharge, erythema, and crusting were assessed using a 4-point scoring system. The subjects were required to report any incident of bleeding or inflammation in between the actual evaluation time points.

Evaluations of sedation were made using a 6-point (0 through 5) sedation scoring system to assess the degree of drowsiness of the subjects after each administration of study drug. Sedation scores were reported by the subject (if awake) as well as by a trained observer using the same rating scale. Subjects were also questioned by the trained observer regarding their degree of drowsiness.

A VAS that consists of a 10 cm (100 mm) horizontal straight line was used to assess acute pain following each administration of study drug. The ends of the scale are defined as extreme limits of pain sensation: 0=no pain, 10=extreme pain. The subjects were asked to mark a point on the scale that best describes their intensity of pain and discomfort. The location of the marking at each time point was measured and noted as the reported score.

Blood samples for the measurement of plasma concentrations of diazepam were collected. Plasma samples from all subjects that complete at least 1 period of the study were analyzed.

Results

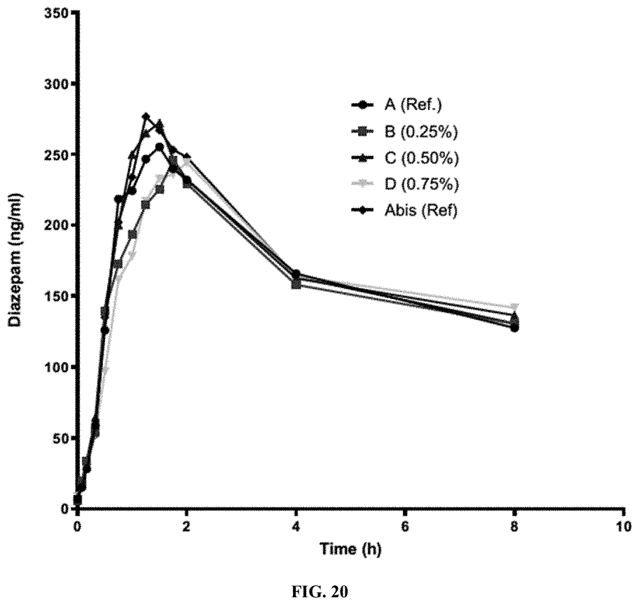

Preliminary PK results are shown in Table 21 and FIG. 20.

TABLE 21

| | Formulations | | | | |
|---|---|---|---|---|---|
| | A | B | C | D | Abis |
| No. of Peaks | 1 | 1 | 1 | 1 | 1 |
| Cₘₐₓ | 255.2 | 245.7 | 272.3 | 244.2 | 276.6 |
| Tₘₐₓ | 1.500 | 1.750 | 1.500 | 2.000 | 1.250 |

Cₘₐₓ = peak concentration of diazepam (ng/mL)
Tₘₐₓ = time of Cₘₐₓ in hours
Abis = Second Formulation A dose

The results show that the single dose Formulation C, with 20% (w/v) of diazepam and 0.50% (w/v) of the alkyl maltoside n-dodecyl beta D-maltoside (DDM) had a comparable Cₘₐₓ and Tₘₐₓ to the reference Formulation A, the 2 nasal spray actuations of the current formulation of Valtoco. Surprisingly, Formulation C, with 0.50% (w/v) of the alkyl maltoside n-dodecyl beta D-maltoside (DDM) had a higher Cₘₐₓ and faster Tₘₐₓ to the test Formulation B, with 0.25% (w/v) of the alkyl maltoside n-dodecyl beta D-maltoside (DDM) and Formulation D, with 0.75% (w/v) of the alkyl maltoside n-dodecyl beta D-maltoside (DDM).

EXAMPLE 25: Clinical Study on the Duration of Intranasal Diazepam Seizure Activity Suppression: Responsive Neurostimulation (RNS) is a device approved by FDA as a treatment for refractory focal epilepsy in 2013. The device consists of electrodes that both record and stimulate when electrographic seizures or electrographic spikes of pre-determined characteristics, i.e., Detections, are recorded. The device also keeps a long-term record via continuous ambulatory intracranial electrode monitoring of various types of Detections, stimulations, and more complex Detections called “Long Episodes”, which are Detections that exceed a pre-determined duration, such as longer than 10 seconds, longer than 20 seconds, longer than 30 seconds, longer than 50 seconds, etc. The NeuroPace company has developed data structures and software tools for download and sharing of RNS data, via the Patient Data Management System (PDMS) that is used in the routine management of patients with RNS devices. The RNS data via PDMS is used passively in this study to measure the number and variability of Detections and Long Episodes and to quantify the duration of the anti-seizure effect of diazepam administered intranasally. Detection or stimulation parameters are not changed during the study. Baseline data for six potential study candidates is summarized in Table 22 below.

TABLE 22

| | Implantation | Detection | Areas/Timing |
|---|---|---|---|
| Subject | Location | Rate | of Detection |
| 1 | Bitemporal | 500-1200 times/day | 80:20 favoring right side; |
| | | | occur mostly at night |
| 2 | Mesial temporal | Variable (correlates | 6-7/month |
| | | with seizures) | |
| 3 | Bitemporal | 20-125/hour | — |
| 4 | Bitemporal | 500-2500/day | 80:20 favoring right side; |
| | | | more active during day |
| 5 | Bitemporal | 2000-8000/day | Left side highly favored |
| 6 | Medial | 250-1700/day | — |

Inclusion criteria for study subjects include: (1) Aged 18+; (2) a weight greater than 50 kg; (3) an RNS implant for usual treatment for at least 3 months with Detection settings stable for at least 30 days; (4) no changes in AED dosages, VNS settings (if participant has VNS) or RNS detection or stimulation parameters 30 days before and during the study; (5) a minimum mean rate of Detections of 10 per hour during the hours of 9 AM to 5 PM, and no fewer than 5 Detections in any hour between 9 AM and 5 PM in the 48-hour pre-dose baseline observation period; (6) no more than a 90% change in the highest to lowest hour detection during the 9 AM to 5 PM observation period in the 48-hour pre-dose baseline; (7) focal epilepsy consistent with ILAE criteria supported by either EEG or MRI data and meets ILAE definition of refractory epilepsy; and (8) if on a prescribed dose of selective serotonin reuptake inhibitor (S SRI), serotonin-norepinephrine reuptake inhibitor (SNRI), or an atypical antipsychotic, it must have been stable for at least 3 months.

Exclusion criteria for study subject include: (1) pregnancy; (2) insufficient competency to sign consent; (3) any history of substance abuse within the previous 2 years, with the exception of cannabidiol as EpidiolexR on a stable dose (4); recreational or medicinal use of other forms of marijuana, cannabinoids and/or derivatives in the prior 30 days; (5) history of poor medication compliance as judged by the investigator; (6) any medical or psychiatric condition that the investigator believes would impair reliable participation in the trial; (7) participation in an investigational product/device trial currently or in the preceding 30 days; (8) a diagnosis with only psychogenic or non-epileptic seizures; (9) use of rescue benzodiazepines less than 14 days before baseline detection rate assessment begins (stable doses of a prescribed benzodiazepine for 30 days prior to study entry is permitted); (10) a clinical seizure during the period starting 96 hours before dose is administered until 48 hours after the intranasal diazepam administration; (11) a history of allergy to diazepam or midazolam, or any of the ingredients of Valtoco®; and (12) a history of adverse reaction to diazepam or midazolam that in the opinion of the investigator would jeopardize the health of the participant or interfere with interpretation of study results.

Subjects undergo screening from 7 days to 48 hours prior to planned dosing. A retrospective data collection of the prior 7 days from the RNS Patient Data Management System (PDMS) is undertaken on the day of dosing. Data from the 7-day baseline period is used for comparisons to the post-dose periods. Up to 8 participants (in order to complete 4 participants) who have previously been implanted with an RNS system based on clinical criteria, for a minimum of 3 months, and whose RNS parameters for recording and stimulation have been stable for at least 30 days are enrolled.

Participants receive a single weight-based dose of intranasal diazepam, are observed for 4 hours in the clinic, and then go home. Administration of intranasal diazepam is performed on a weight basis according to prescribing information. For participants weighing 51 kg to 75 kg, 15 mg diazepam is administered as 7.5 mg in each nostril. For participants weighing 76 or more kg, 20 mg diazepam is administered as 10 mg in each nostril. Participants upload RNS data once daily (similar to standard protocol for any RNS patient) for 7 days following their dose of intranasal diazepam. The study coordinator calls to remind the participants to upload daily for the seven days after dosing.

The primary data measurement in the study is detections due to the high number of detections typically recorded in the target subjects. This allows an adequate sample size to measure differences between the 7-day (168 hour) pre-dose baseline and the 48-hour post-dose evaluation period. In a previously published study of long-term prediction of antiepileptic drug (AED) effects based on early response using RNS criteria, during the baseline observation period, the median Long Episode rate was 1 per day (range: 0 to 143), and the median Detection (termed “episode starts” in the study) rate was 750 per day (range: 20 to 5097). (Quraishi, Epilepsia 2019) The main focus of this study is on the most active electrode pair of the available electrodes for individual participants, all of whom have multiple electrodes. However, data from all pairs of RNS electrodes are evaluated.

Two characteristics of the Detection rates for participants in this study are important. First, the baseline Detection rate are sufficient to show any effect of the treatments. The highest Detection rates are seen in patients with mesial temporal lobe (MTL) seizures. In the pivotal studies of RNS, for all bilateral MTL patients in the studies (year 1), the mean Detection rate was 1465 per-day, the median was 828 per-day, the range was 0-4229 per-day, and interquartile range was 92-2352 per-day. Second, the variation from day to day in Detections are not high. In the pivotal studies some participants had higher variability in Detections from one day to another, whereas other participants had more stable Detection rates. The baseline for Detection counting extend to 7 days prior to dosing to account for variations in Detection rates during baseline.

An advantage of using RNS Detections as the main outcome is that all subjects have an extensive baseline recording history, in some cases years. In addition, the baseline and treatment phases are performed the same time of day to control for circadian rhythms of spikes and seizures, which are well described in people with seizures (Baud, Nature Communications 2018).

Long Episodes are also measured because Long Episodes have the best correlation with clinical seizures. An analysis of a large sample of RNS cases shows that if there were no Long Episodes recorded on a given day, the patient was unlikely to report a clinical seizure. On the other hand, the positive predictive value was 42.4%, indicating that not all Long Episodes recorded were reported as seizures. (Tara Skarpaas, NeuroPace, personal communication). Long Episodes that are not reported as clinical seizures may have been subclinical, occurred during sleep or unwitnessed and forgotten by the patient, or possibly false positive Detections.

Data is collected on the percent change in Detections calculated separately for each hour during the first 8 hours of the post-dose observation period. The number of Detections during each hour of the 8-hour period post dose is compared to the same hour during the seven comparable 8-hour sessions of the 7-day pre-dose observation period. That is, if the dose was given at 9 AM on day 0, then the mean number of Detections at baseline is calculated as the mean number of Detections from 9 AM to 10 AM on days-1 through day-7. The percent change in Detections is calculated as (number of Detections in hour 1 post dose)/(mean number of Detections in hour 1 on Day-1 through and Day-7). The same hourly change is calculated for each of the 8 hours after dosing. The comparison is the same hours each day to minimize the hour to hour variability typically seen in these measures.

Using “detailed diagnostics,” visual analysis of minute by minute Detections is reviewed for obvious patterns of interest for the 8 hours post dosing. Patterns that are identified include, but are not limited to (1) the total number of hours during the 48-hour post-dose observation period where the number of Detections is <50% of the mean hourly Detection rate over the 7-day pre-dose observation period; (2) the number of long episodes recorded during the 7-day pre-dose observation period compared to the number of long episodes during the post-dose 48-hour observation period; (3) characteristics of the long episodes like duration, frequency, and morphology; (4) number of diary-recorded seizures during the post-dose observation period; (5) quantification of the 2 highest hourly detection rates for the 48-hour pre-dose and 48-hour post-dose periods; (6) the number of days required for the daily detection rate to fall within the 90% confidence interval of the 9 am to 5 PM baselines from the 7-day pre-dose baseline; and (7) the time course of observable changes in beta activity on the ECoG recorded samples on the day of dosing.

Descriptive statistics are used to tabulate and summarize study outcomes. Continuous variables are summarized by descriptive statistics (sample size, mean and standard deviation, median, minimum and maximum). Discrete variables are summarized by frequencies and percentages. Adverse events are summarized by presenting the number and percentage of patients having any adverse event. Any statistical tests performed to explore the data are used only to highlight any interesting comparisons that may warrant further consideration.

Data regarding treatment-emergent adverse events (TEAEs) are collected in this study. TEAEs are events that are not present at baseline, or if present at baseline, have worsened in severity. AEs are assessed post-dosing of the intranasal diazepam and until the follow-up telephone contact has been completed. Any AE reported by the subject or noted by the Investigator or his/her designee is recorded on the case report form (CRF) regardless of the Investigator opinion of causality. The following information is recorded for each AE: description of the event, date and time of onset, date and time of resolution, severity, causal relationship to study drug, outcome, action taken with the study drug and any treatment given. All clinically significant abnormal changes from baseline in physical examination findings, vital signs, is collected, graded with regards to severity or clinical significance, assessed for causal relationship and recorded on the CRF.

AEs in the CRF are classified according to the most recent FDA definitions and in a manner consistent with International Conference on Harmonization (ICH) guidelines. As such the following definitions are used:

An AE is any unfavorable and unintended sign, symptom, or disease temporally associated with the use of an investigational (medicinal) product (IP) or other protocol-imposed intervention, regardless of attribution. An AE may include intercurrent illnesses or injuries that represent an exacerbation (increase in frequency, severity, or specificity) of pre-existing conditions (e.g., worsening of asthma). A laboratory abnormality is reported on the “Adverse Event” case report form only if it is associated with clinical sequelae or requires therapeutic intervention. Whenever possible, it is preferable to record a diagnosis as the AE term rather than a series of symptoms relating to a diagnosis. AEs are coded according to the Medical Dictionary for Regulatory Activities (MedDRA) and graded according to the Common Terminology Criteria for Adverse Events (CTCAE) v5.0 (Appendix C).

The reporting period for non-serious AEs starts after the first administration of study drug on Day 0 and ends when the telephone contact on Day 2 is completed.

If an AE remains unresolved at the telephone contact on Day 2, the subject is followed, at the Investigator's discretion, until resolution of the event. SAEs are followed until resolution by the PI, even if this extends beyond the study-reporting period. Resolution is defined as the return to baseline status or stabilization of the condition with the expectation that it will remain chronic.

The Investigator assesses AEs for severity, relationship to IP, and as to whether the event meets one or more of the definitions of an SAE. The assessments are recorded on the source documents and AE CRF, using the categories defined below in Table 23.

TABLE 23

| Causality | |
|---|---|
| Category | Description |
| Unlikely | A clinical event, including laboratory test abnormality, |
| | with a temporal relationship to drug administration |
| | which makes a causal relationship improbable, |
| | and in which other drugs, chemicals or underlying |
| | disease provide plausible explanations. |
| | For the purpose of this protocol, the term unlikely is considered |
| | not related to study medication and an “Adverse Event”. |
| Possible | A clinical event, including laboratory test abnormality, |
| | with a reasonable time sequence to administration |
| | of the drug, but which could also be explained by |
| | concurrent disease or other drugs or chemicals. |
| | Information on drug withdrawal may be lacking or unclear. |
| | For the purpose of this protocol, an event that has |
| | possible relationship to study medication is defined as a |
| | “Suspected Adverse Drug Reaction”. |
| Probable | A clinical event, including laboratory test abnormality, |
| | with a reasonable time sequence to administration |
| | of the drug, unlikely to be attributed to concurrent |
| | disease or other drugs or chemicals, and which follows |
| | a clinically reasonable response on withdrawal. |
| | For the purpose of this protocol, an event that has |
| | probable relationship to study medication is |
| | defined as an “Adverse Drug Reaction”. |

In order to classify adverse events and diseases, preferred terms are assigned by the sponsor or its designee to the original terms entered on the CRF, using MedDRA.

For those AEs that are not described on the CTCAE v 5.0, such AEs are graded on a 3-point scale (mild, moderate, severe) and reported as indicated on the CRF. Intensity of such an AE is defined as follows in Table 24.

TABLE 24

| Severity | | |
|---|---|---|
| Assessment | | |
| Terminology | | |
| for Reporting | | |
| Adverse Events | | |
| (CTCAE v 5.0) | Common | |
| CTCAE Grade | Term | Description |
| 1 | Mild | Mild; asymptomatic or |
| | | mild symptoms; clinical |
| | | or diagnostic observations only; |
| | | intervention not indicated. |
| 2 | Moderate | Moderate; minimal, local or |
| | | noninvasive intervention indicated; |
| | | limiting age-appropriate |
| | | instrumental ADL. |
| 3 | Severe | Severe or medically significant but not |
| | | immediately life-threatening; |
| | | hospitalization or prolongation |
| | | of hospitalization indicated; |
| | | disabling; limiting self-care ADL. |

According to the ICH Guidelines for Good Clinical Practice (E6), an SAE is any untoward medical occurrence during the course of a clinical investigation that is characterized by one or more of the following:
Results in death
Is life-threatening
Requires in-subject hospitalization or prolongation of existing hospitalization
Results in persistent or significant disability/incapacity
Is a congenital anomaly/birth defect
Important medical events

Safety: Descriptive statistics are provided for actual values and change from baseline values for neurological examination, vital signs, and SSS assessments. The incidence and severity of TEAEs reported during the study and their relationship to study drug are tabulated. TEAEs are coded using the MedDRA and are presented by body system. The World Health Organization Drug Dictionary (WHODD) is used to classify concomitant medications by therapeutic class and preferred term. Concomitant medication usage is summarized by the number and percentage of subjects receiving each medication within each therapeutic class.

Assessment of Sleepiness: Objective evaluations of sleepiness are made using the SSS 7-point (1→7), shown below in Table 25, to assess the degree of drowsiness of the subjects after administration of study drug. Sleepiness scores are reported by the subject as well as by a trained observer, using the same rating scale, prior to dosing (baseline) and at 15, 30, 45, 60, 75, 90, 105, 120, 150, 180, and 240 minutes after dosing. The SSS is also administered prior to discharge from the clinic to ensure return to baseline. Subjects are not allowed to operate a vehicle or any machinery for 48 hours after discharge.

TABLE 25

| Degree of Sleepiness | Scale Rating |
|---|---|
| Feeling active, vital, alert, or wide awake | 1 |
| Functioning at high levels, but not fully alert | 2 |
| Awake, but relaxed; responsive but not fully alert | 3 |
| Somewhat foggy, let down | 4 |
| Foggy; losing interest in remaining awake; slowed down | 5 |
| Sleepy, woozy, fighting sleep; prefer to lie down | 6 |
| No longer fighting sleep, sleep onset soon; | 7 |
| having dream-like thoughts | |
| Asleep | X |

Columbia Suicide Severity Rating Scale: The C-SSRS (baseline-screening version) is administered at pre-dose baseline. The C-SSRS (since last visit version) is administered 4 hours after the dose of intranasal diazepam prior to discharge from the clinic.

Electrographic detection of spikes and electrographic seizures with intracortical electrodes provide a surrogate to seizures and provide a more rapid and efficient measure of duration of anti-seizure effect, i.e., a measure of pharmacodynamic effect. Although not as definitive as measuring actual seizures, the findings indirectly test the hypothesis that the pharmacokinetic half-life translates into longer lasting anti-seizure effect for intranasal diazepam.

The study described is a Pilot 1 using intranasal diazepam only, to provide a proof of concept that intranasal administration of intranasal diazepam is capable of measurable reduction of RNS Detections over the time frame of the study. A Pilot 2 study could determine if intranasal midazolam provides a measurable degree of Detection reduction in a similar sized population. Such a study may be a 4 period, replicate, crossover, blinded design with washout of 4 weeks between treatments to compare intranasal diazepam with intranasal midazolam for magnitude and duration of Detection suppression in people with RNS.

EXAMPLE 26: Increase in Interseizure Cluster Interval (ISCI) in Patients with Seizure Clusters Treated with Diazepam Nasal Spray. Non-intravenous benzodiazepines are key rescue therapies for treatment of outpatient seizure clusters in patients with epilepsy. Seizure clusters, or acute repetitive seizures, are emergencies associated with increased risk of prolonged seizures and status epilepticus, requiring prompt treatment to lower the risk of associated morbidities. Diazepam nasal spray is approved for acute treatment of seizure clusters in patients with epilepsy aged ≥6 years. In this study, seizure data was used to understand seizure cluster patterns both individually and across the subject cohort by examining the time between doses of diazepam nasal spray (Valtoco®).

The study enrolled patients aged 6-65 years with frequent seizure clusters. Care partners and patients were trained to administer doses of 5, 10, 15, or 20 mg of diazepam nasal spray based on age (≤11 or ≥12 years) and weight, with instructions to administer second doses 4-12 hours later if needed. Investigators could adjust doses for effectiveness or safety reasons. Seizures, drug administration, and TEAEs were recorded in seizure diaries. ISCIs are measured as the time, in days, between administration of the diazepam nasal spray for the treatment of a seizure cluster.

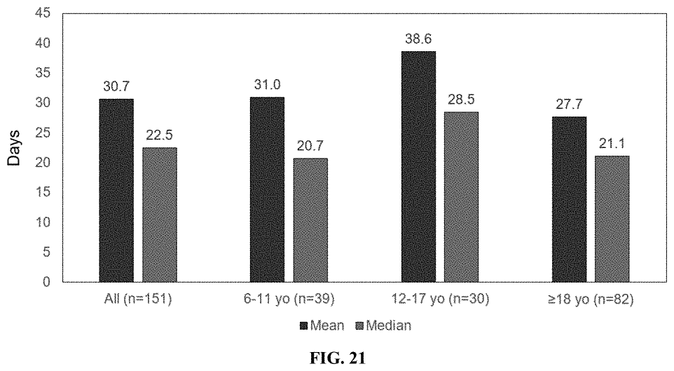

The study enrolled 175 subjects. 163 subjects received at least one dose of the diazepam nasal spray, out of which 151 had at least one ISCI (i.e., at least two seizure clusters). Out of those 151 subjects the mean and median ISCI was relatively uniform across all age groups. All 151 subjects had a mean ISCI of 30.7 days with a median of 22.5 days; 6-11 year old subjects (n=39) had a mean ISCI of 31.0 days with a median of 20.7 days; 12-17 year old subjects (n=30) had a mean ISCI of 38.6 days with a median of 28.5 days; and subjects greater than or older than 18 (n=82) had a mean ISCI of 27.7 days with a median of 21.1 days (FIG. 21). Thus, subject age does not seem to affect ISCI duration.

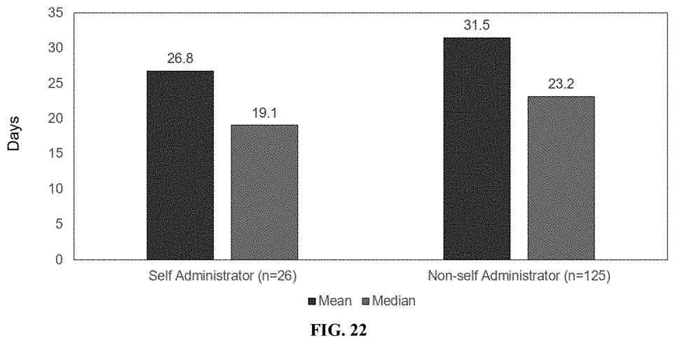

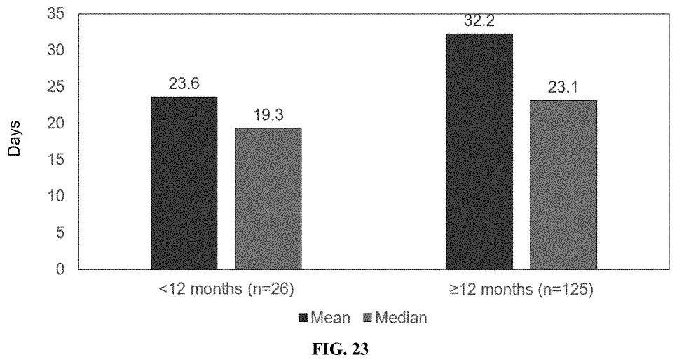

Interestingly however, whether the subject self-administered the medication and the length of time the patient was active in the study did have an impact on ISCI duration. For those subjects who self-administered the diazepam nasal spray, the mean ISCI was 26.8 days with a median of 19.1 days (n=26). However, when the subjects did not self-administer, i.e., a caretaker or helper routinely administered the nasal spray to make sure it was used properly, the mean ISCI increased to 31.5 days with a median of 23.2 days (n=125) (FIG. 22). For those subjects who were active in the study for less than 12 months, the mean ISCI was 23.6 days with a median of 19.3 days (n=26). Meanwhile, those subjects who were active in the study at least 12 months, the mean ISCI was 32.2 days with a median of 23.1 days (n=125) (FIG. 23). Thus, it is possible that ISCI duration is improved simply by properly and effectively using the diazepam nasal spray over a longer period of time.

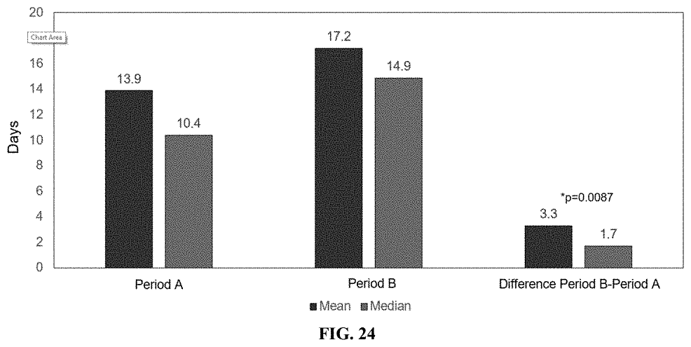

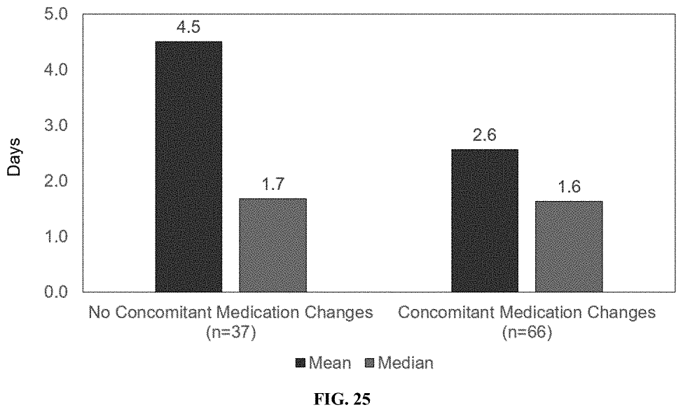

To analyze changes in ISCI duration over time, data from the first three months of the study (Period A) was compared to data from the last three months of the study (Period B). A total of 103 subjects had at least one ISCI in both Period A and Period B. For Period A, the mean ISCI was 13.9 days and the median was 10.4 days. However, for Period B, the mean ISCI increased to 17.2 days with a median of 14.9 days. This results in a statistically significant increase of a mean 3.3 days and a median of 17 days (p=0.0087, FIG. 24). This is a surprising result which indicates that the use of diazepam nasal sprays can not only treat seizure clusters, but actually increases the ISCI duration, resulting in fewer subject seizures with continued use. To help confirm that this result is accurate and not the result of changing daily medications, subjects were split out by whether or not they had concomitant medication changes during the study. Concomitant medication changes can be common in longer duration studies and could possibly impact the ISCI duration. However, subjects still saw an increase in ISCI duration regardless of whether or not they had concomitant medication changes. The no concomitant changes group saw a mean ISCI increase from Period A to B of 4.5 days with a median of 1.7 days (n=37), while the concomitant changes group saw a mean ISCI increase of 2.6 days with a median of 1.6 days (n=66) (FIG. 25). Thus, the use of the diazepam nasal spray and not changes to other concomitant medications appear to be the case of the increase ISCI durations.

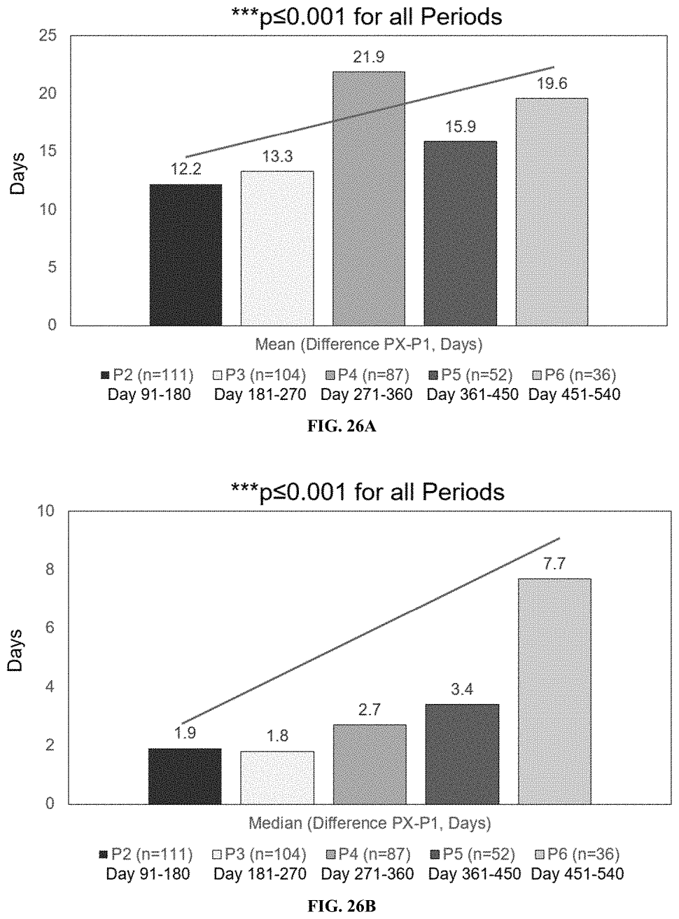

To further analyze changes in ISCI duration over time, a multiple period sensitivity analysis was performed. Period 1 was defined as the first three months of the study (Days 1-90), which each new Period being the next consecutive three months: Period 2 (Days 91-180, n=111); Period 3 (Days 181-270, n=104); Period 4 (Days 271-360, n=87)); Period 5 (Days 361-450, n=52); and Period 6 (Days 451-540, n=36). Periods 2-6 were compared to Period 1 to determine the change in ISCI duration as the study progressed. The results show that ISCI duration generally increases as time goes on. Period 2 has a mean ISCI difference of 12.2 days and a median of 1.9 days compared to Period 1; Period 3 has a mean ISCI difference of 13.3 days and a median of 1.8 days compared to Period 1; Period 4 has a mean ISCI difference of 21.9 days and a median of 2.7 days compared to Period 1; Period 5 has a mean ISCI difference of 15.9 days and a median of 3.4 days compared to Period 1; and Period 6 has a mean ISCI difference of 19.6 days and a median of 7.7 days compared to Period 1 (p≤0.001, FIGS. 26A and 26B). To put these results in context using Period 6 results as an example, a mean ISCI increase of 19.6 days equals about 4 to 5 fewer seizure clusters per year.

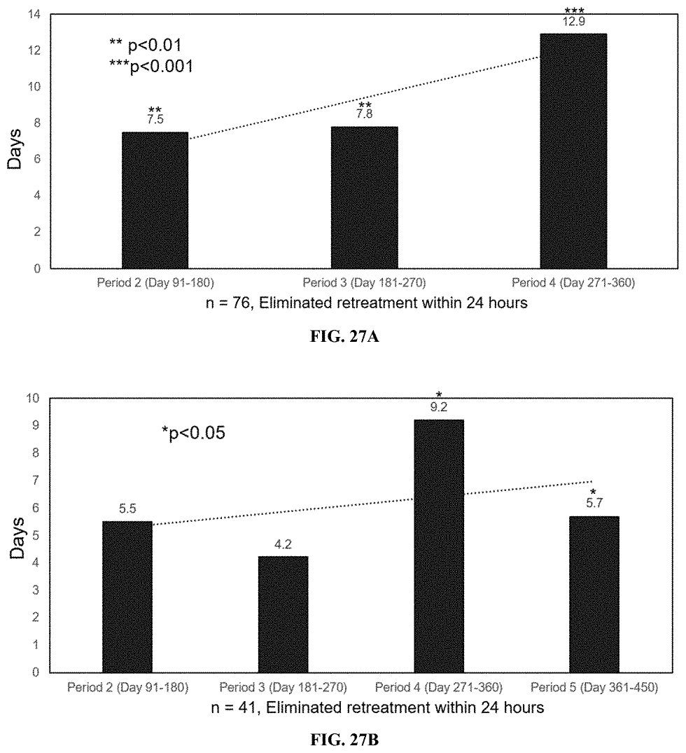

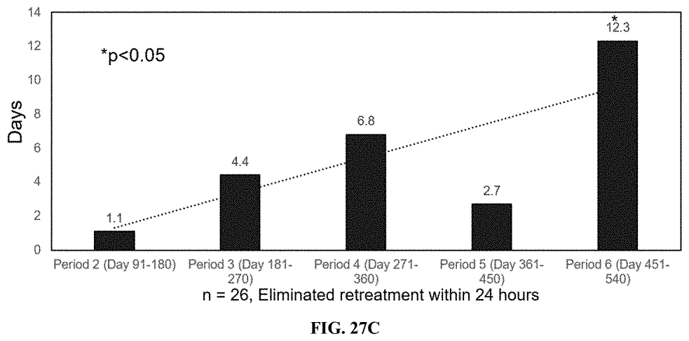

The multiple period sensitivity analysis can be further refined by using the consistent cohort: the group of same subjects who provided data through Periods 1-4, 1-5, or 1-6, and elimination of any retreatment data within 24 hours. Periods 1-6 duration were the same as listed above, and Periods 2-6 were again compared to Period 1 to determine the change in ISCI duration as the study progressed for each group. For Periods 1-4 (n=76), Period 2 has a mean ISCI difference of 7.5 days from Period 1, Period 3 has a mean ISCI difference of 7.8 days from Period 1, and Period 4 has a mean ISCI difference of 12.9 days from Period 1 (FIG. 27A). For Periods 1-5 (n=41), Period 2 has a mean ISCI difference of 5.5 days from Period 1, Period 3 has a mean ISCI difference of 4.2 days from Period 1, Period 4 has a mean ISCI difference of 9.2 days from Period 1, and Period 5 has a mean ISCI difference of 5.7 days from Period 1 (FIG. 27B). For Periods 1-6 (n=26), Period 2 has a mean ISCI difference of 1.1 days from Period 1, Period 3 has a mean ISCI difference of 4.4 days from Period 1, Period 4 has a mean ISCI difference of 6.8 days from Period 1, Period 5 has a mean ISCI difference of 2.7 days from Period 1, and Period 6 has a mean ISCI difference of 12.3 days from Period 1 (FIG. 27C). Collectively, these results continue to show that treatment improves control of seizure cluster frequency over time.

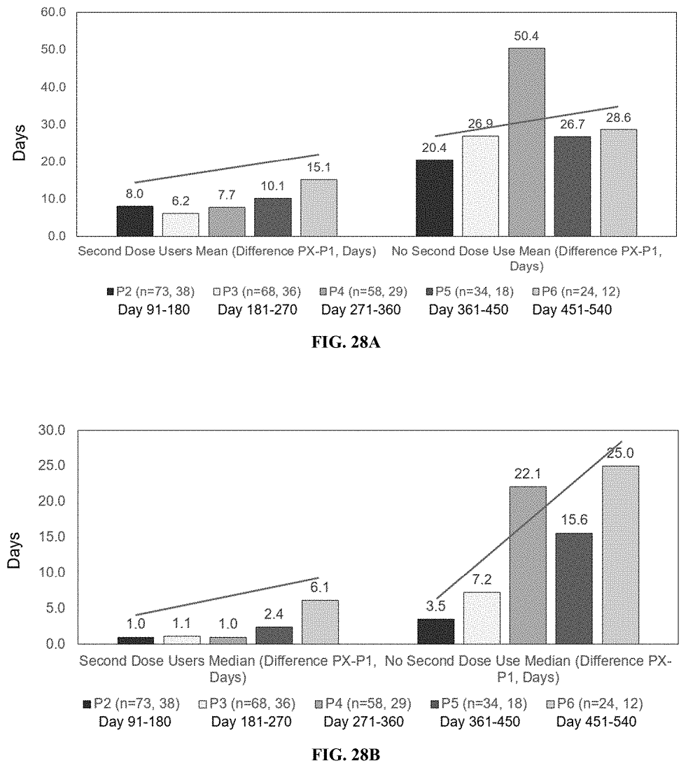

As a final analysis, the multiple period sensitivity analysis data was further separated into subjects that only needed a single dose of the diazepam nasal spray to treat their seizures and subjects that had seizures severe enough to necessitate a second dose. Periods 1-6 duration were the same as listed above, and Periods 2-6 were again compared to Period 1 to determine the change in ISCI duration as the study progressed for each group. For the second dose subjects, Period 2 (n=73) has a mean ISCI difference of 8.0 days and a median of 1.0 days compared to Period 1; Period 3 (n=68) has a mean ISCI difference of 6.2 days and a median of 1.1 days compared to Period 1; Period 4 (n=58) has a mean ISCI difference of 7.7 days and a median of 1.0 days compared to Period 1; Period 5 (n=34) has a mean ISCI difference of 10.1 days and a median of 2.4 days compared to Period 1; and Period 6 (n=24) has a mean ISCI difference of 15.1 days and a median of 6.1 days compared to Period 1. For the no second dose subjects, Period 2 (n=38) has a mean ISCI difference of 20.4 days and a median of 3.5 days compared to Period 1; Period 3 (n=36) has a mean ISCI difference of 26.9 days and a median of 7.2 days compared to Period 1; Period 4 (n=29) has a mean ISCI difference of 50.4 days and a median of 22.1 days compared to Period 1; Period 5 (n=18) has a mean ISCI difference of 26.7 days and a median of 15.6 days compared to Period 1; and Period 6 (n=24) has a mean ISCI difference of 28.6 days and a median of 25.0 days compared to Period 1 (collectively FIGS. 28A and 28B). These results show that regardless of whether or not subjects required a second dose of the diazepam nasal spray to treat their seizures, ISCI duration increased over the course of the study.

1. A method of rapidly treating a seizure in a subject suffering from recurrent seizures, the method comprising administering to nasal mucosal membrane of the subject a composition comprising:
an effective amount of diazepam or a pharmaceutically acceptable salt thereof;
n-dodecyl beta-D-maltoside;
one or more natural or synthetic tocopherols or tocotrienols in an amount from about 30% w/v to about 95% w/v; and
one or more alcohols in an amount from about 25% w/v to about 45% w/v, wherein administration of the composition rapidly reduces the severity of the seizure.

2. The method of claim 1, wherein the administration is during the ictal phase of the seizure.

3. The method of claim 1, wherein the administration shortens the length of the seizure.

4. The method of claim 1, wherein the administration stops the seizure.

5. The method of claim 1, wherein the reduction in severity occurs within about 2 minutes of administration.

6. The method of claim 1, wherein the seizure is an absence seizure, a myoclonic seizure, a clonic seizure, a tonic seizure, a tonic-clonic seizure, an atonic seizure, a focal seizure, or any combination thereof.

7. The method of claim 1, wherein the seizure is a seizure within a seizure cluster or acute repetitive seizures.

8. The method of claim 1, wherein the administration is performed by a caregiver.

9. The method of claim 1, wherein the administration is performed by the subject.

10. The method of claim 1, wherein the effective amount of diazepam is about 5 mg to about 20 mg diazepam in a volume of about 10 μL to 200 μL of the composition.

11. The method of claim 1, wherein the n-dodecyl beta-D-maltoside is in a concentration of about 0.25% w/v to about 0.75% w/v.

12. The method of claim 1, wherein the one or more natural or synthetic tocopherols or tocotrienols is selected from the group consisting of α-tocopherol, β-tocopherol, γ-tocopherol, δ-tocopherol, α-tocotrienol, β-tocotrienol, γ-tocotrienol, δ-tocotrienol, tocophersolan, and any combinations thereof.

13. The method of claim 1, wherein the one or more alcohols is a combination of ethanol and benzyl alcohol, or only benzyl alcohol.

14. The method of claim 1, wherein the composition consists of:
an effective amount of diazepam or a pharmaceutically acceptable salt thereof;
n-dodecyl beta-D-maltoside in an amount from about 0.25% w/v to about 0.75% w/v;
vitamin E in an amount from about 35% w/v to about 45% w/v; and
benzyl alcohol in an amount from about 35% w/v to about 45% w/v.

15. A method of modulating the beta frequency in a subject suffering from recurrent seizures, the method comprising administering to nasal mucosal membrane of the subject during the ictal phase of a seizure a composition comprising:
an effective amount of diazepam or a pharmaceutically acceptable salt thereof;
n-dodecyl beta-D-maltoside;
one or more natural or synthetic tocopherols or tocotrienols in an amount from about 30% w/v to about 95% w/v; and
one or more alcohols in an amount from about 25% w/v to about 45% w/v, wherein administration of the composition modulates the beta frequency of the subject.

16. The method of claim 15, wherein the administration is during the ictal phase of the seizure.

17. The method of claim 15, wherein the administrations reduces the severity of the seizure.

18. The method of claim 15, wherein the administration stops the seizure.

19. The method of claim 15, wherein the beta frequency modulation occurs within about 6 minutes of administration.

20. The method of claim 15, wherein the seizure is an absence seizure, a myoclonic seizure, a clonic seizure, a tonic seizure, a tonic-clonic seizure, an atonic seizure, a focal seizure, or any combination thereof.

21. The method of claim 15, wherein the seizure is a seizure within a seizure cluster or acute repetitive seizures.

22. The method of claim 15, wherein the administration is performed by a caregiver.

23. The method of claim 15, wherein the administration is performed by the subject.

24. The method of claim 15, wherein the effective amount of diazepam is about 5 mg to about 20 mg diazepam in a volume of about 10 μL to 200 μL of the composition.

25. The method of claim 15, wherein the n-dodecyl beta-D-maltoside is in a concentration of about 0.25% w/v to about 0.75% w/v.

26. The method of claim 15, wherein the one or more natural or synthetic tocopherols or tocotrienols is selected from the group consisting of α-tocopherol, β-tocopherol, γ-tocopherol, δ-tocopherol, α-tocotrienol, β-tocotrienol, γ-tocotrienol, δ-tocotrienol, tocophersolan, and any combinations thereof.

27. The method of claim 15, wherein the one or more alcohols is a combination of ethanol and benzyl alcohol, or only benzyl alcohol.

28. The method of claim 15, wherein the composition consists of:
an effective amount of diazepam or a pharmaceutically acceptable salt thereof;
n-dodecyl beta-D-maltoside in an amount from about 0.25% w/v to about 0.75% w/v;
vitamin E in an amount from about 35% w/v to about 45% w/v; and
benzyl alcohol in an amount from about 35% w/v to about 45% w/v.

29. A method of treating or preventing seizures in a subject suffering from recurrent seizures, the method comprising administering to nasal mucosal membrane of the subject a composition comprising:
an effective amount of diazepam or a pharmaceutically acceptable salt thereof;
n-dodecyl beta-D-maltoside;
one or more natural or synthetic tocopherols or tocotrienols in an amount from about 30% w/v to about 95% w/v; and
one or more alcohols in an amount from about 25% w/v to about 45% w/v, wherein the subject experiences a reduced incidence or severity of somnolence after the administration.

30. The method of claim 29, wherein the composition is administered to the subject before, during, or after a seizure.

31. The method of claim 29, wherein the reduced incidence or severity of somnolence is in comparison with somnolence experienced after administration of a therapeutically effective amount of diazepam via rectal, intravenous, or oral administration.

32. The method of claim 29, wherein the seizure is an absence seizure, a myoclonic seizure, a clonic seizure, a tonic seizure, a tonic-clonic seizure, an atonic seizure, a focal seizure, or any combination thereof.

33. The method of claim 29, wherein the seizure is a seizure within a seizure cluster or acute repetitive seizures.

34. The method of claim 29, wherein the administration is performed by a caregiver.

35. The method of claim 29, wherein the administration is performed by the subject.

36. The method of claim 29, wherein the effective amount of diazepam is about 5 mg to about 20 mg diazepam in a volume of about 10 μL to 200 μL of the composition.

37. The method of claim 29, wherein the n-dodecyl beta-D-maltoside is in a concentration of about 0.25% w/v to about 0.75% w/v.

38. The method of claim 29, wherein the one or more natural or synthetic tocopherols or tocotrienols is selected from the group consisting of α-tocopherol, β-tocopherol, γ-tocopherol, δ-tocopherol, α-tocotrienol, β-tocotrienol, γ-tocotrienol, δ-tocotrienol, tocophersolan, and any combinations thereof.

39. The method of claim 29, wherein the one or more alcohols is a combination of ethanol and benzyl alcohol, or only benzyl alcohol.

40. The method of claim 29, wherein the composition consists of:
an effective amount of diazepam or a pharmaceutically acceptable salt thereof;
n-dodecyl beta-D-maltoside in an amount from about 0.25% w/v to about 0.75% w/v;
vitamin E in an amount from about 35% w/v to about 45% w/v; and
benzyl alcohol in an amount from about 35% w/v to about 45% w/v.

41. A method of increasing the time to a second seizure in a subject suffering from recurrent seizures, the method comprising administering to nasal mucosal membrane of the subject a composition comprising:
an effective amount of diazepam or a pharmaceutically acceptable salt thereof;
n-dodecyl beta-D-maltoside;
one or more natural or synthetic tocopherols or tocotrienols in an amount from about 30% w/v to about 95% w/v; and
one or more alcohols in an amount from about 25% w/v to about 45% w/v, wherein administration of the composition increases the time to a second seizure in the subject.

42. The method of claim 41, wherein the composition is administered to the subject before, during, or after a seizure within a seizure cluster or acute repetitive seizures.

43. The method of claim 41, wherein the method comprises administering at least two or more doses of the composition.

44. The method of claim 41, wherein the time to a second seizure is at least 8 hours after the administration of the composition.

45. The method of claim 41, wherein the method precludes the need for a second administration of the composition within at least 24 hours of the first administration of the composition.

46. The method of claim 41, wherein the seizure is an absence seizure, a myoclonic seizure, a clonic seizure, a tonic seizure, a tonic-clonic seizure, an atonic seizure, a focal seizure, or any combination thereof.

47. The method of claim 41, wherein the administration is performed by a caregiver.

48. The method of claim 41, wherein the administration is performed by the subject.

51. The method of claim 41, wherein the effective amount of diazepam is about 5 mg to about 20 mg diazepam in a volume of about 10 μL to 200 μL of the composition.

52. The method of claim 41, wherein the n-dodecyl beta-D-maltoside is in a concentration of about 0.25% w/v to about 0.75% w/v.

53. The method of claim 41, wherein the one or more natural or synthetic tocopherols or tocotrienols is selected from the group consisting of α-tocopherol, β-tocopherol, γ-tocopherol, δ-tocopherol, α-tocotrienol, β-tocotrienol, γ-tocotrienol, δ-tocotrienol, tocophersolan, and any combinations thereof.

54. The method of claim 41, wherein the one or more alcohols is a combination of ethanol and benzyl alcohol, or only benzyl alcohol.

55. The method of claim 41, wherein the composition consists of:
an effective amount of diazepam or a pharmaceutically acceptable salt thereof;
n-dodecyl beta-D-maltoside in an amount from about 0.25% w/v to about 0.75% w/v;
vitamin E in an amount from about 35% w/v to about 45% w/v; and
benzyl alcohol in an amount from about 35% w/v to about 45% w/v.

56. A method of increasing the time to a seizure cluster in a subject suffering from recurrent seizure clusters, the method comprising administering to nasal mucosal membrane of the subject a composition comprising
an effective amount of diazepam or a pharmaceutically acceptable salt thereof;
n-dodecyl beta-D-maltoside;
one or more natural or synthetic tocopherols or tocotrienols in an amount from about 30% w/v to about 95% w/v; and
one or more alcohols in an amount from about 25% w/v to about 45% w/v, wherein administration of the composition increases the length of an interseizure cluster interval (ISCI) in the subject.

57. The method of claim 56, wherein the composition is administered to the subject before, during, or after a seizure within a seizure cluster or acute repetitive seizures.

58. The method of claim 56, wherein the method comprises administering at least two or more doses of the composition.

59. The method of claim 56, wherein the subject has a decrease in the frequency of seizure clusters.

60. The method of claim 56, wherein the ISCI duration increases by at least 1, 2, 3, 4, 5, 6, 7, 8, 9, 10, 11, 12, 13, 14, 15, 16, 17, 18, 19, or 20 days.

61. The method of claim 56, wherein the seizure is an absence seizure, a myoclonic seizure, a clonic seizure, a tonic seizure, a tonic-clonic seizure, an atonic seizure, a focal seizure, or any combination thereof.

62. The method of claim 56, wherein the administration is performed by a caregiver.

63. The method of claim 56, wherein the administration is performed by the subject.

64. The method of claim 56, wherein the effective amount of diazepam is about 5 mg to about 20 mg diazepam in a volume of about 10 μL to 200 μL of the composition.

65. The method of claim 56, wherein the n-dodecyl beta-D-maltoside is in a concentration of about 0.25% w/v to about 0.75% w/v.

66. The method of claim 56, wherein the one or more natural or synthetic tocopherols or tocotrienols is selected from the group consisting of α-tocopherol, β-tocopherol, γ-tocopherol, δ-tocopherol, α-tocotrienol, β-tocotrienol, γ-tocotrienol, δ-tocotrienol, tocophersolan, and any combinations thereof.

67. The method of claim 56, wherein the one or more alcohols is a combination of ethanol and benzyl alcohol, or only benzyl alcohol.

68. The method of claim 56, wherein the composition consists of:
an effective amount of diazepam or a pharmaceutically acceptable salt thereof;
n-dodecyl beta-D-maltoside in an amount from about 0.25% w/v to about 0.75% w/v;
vitamin E in an amount from about 35% w/v to about 45% w/v; and
benzyl alcohol in an amount from about 35% w/v to about 45% w/v.
